(19) United States
(12) Patent Application Publication
(10) Pub. No.: US 2010/0092456 A1
(43) Pub. Date: Apr. 15, 2010

(54) METHODS OF TREATMENT

(76) Inventors: Louise Bisset, London (GB); Tim Hammonds, London (GB); Nullin Divecha, Manchester (GB); Jonathan Halstead, Basel (CH)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/302,405

(22) PCT Filed: May 31, 2007
(86) PCT No.: PCT/GB2007/002035

§ 371 Date: Oct. 13, 2009

(30) Foreign Application Priority Data
May 31, 2006 (GB) 0610778.3

(51) Int. Cl. A61K 39/395; C12Q 1/48; A61K 31/7088

(52) U.S. Cl. 424/130.1; 435/15; 435/29; 514/44 A

(57) ABSTRACT

Methods for modulating apoptosis and neurite outgrowth in cells by modulating levels of PtdIns(4,5)P₂. Agents for such modulation, including agents which inhibit or activate type I and type II PIP kinases, and methods for identifying such agents. Methods for treating or preventing hyperproliferative disorders and neurodegenerative diseases using such agents.

### FIELD

This invention is related to the field of treatment of conditions such as hyperproliferative disorders, neurodegenerative disorders and cardiac reperfusion. The invention relates further to methods of identifying compounds, which are useful in the method of the present invention.

### BACKGROUND

A class of compounds which have long been known to be associated with intracellular signalling are Phosphatidylinositol lipids (PtdIns). Phosphatidylinositol contains a head group with five free hydroxyl groups of which three have been found to be phosphorylated in vivo: position D-3, D-4 and D-5. In total, seven phosphorylated PtdIns have been identified: three monophosphates (PtdInsP), three bisphosphates (PtdInsP2) and one trisphosphate (PtdIns(3,4,5)P₃). The monophosphate PtdIns4P is a precursor of PtdIns(4,5)P₂ and thought to be involved in regulating actin binding proteins. It is believed that PtdIns4P constitutes 93% of the total cellular PtdInsP. PtdIns3P is found at low concentrations within the cell and is considered to be involved in membrane trafficking. PtdIns5P is another precursor of PtdIns(4,5)P₂, however it constitutes only 2% of the cellular PtdinsP. Three double phosphorylated PtdInsP₂ are known of which PtdIns(4,5)P₂ is the most investigated example. Two mechanisms for the synthesis of PtdIns(4,5)P₂ by PtdInsP phosphorylation have been identified, D-5 phosphorylation of PtdIns4P and D-4 phosphorylation of PtdIns5P. The kinases which facilitate the phosphorylation of these PtdInsPs are named phosphatidylinositolphosphate kinases (PIPkins). The PIP kinases represent a large enzyme family and are divided into three types: type I, type II and type III. Type I PIP kinase is thought to be responsible for the majority of PtdIns(4,5)P₂ synthesis by phosphorylating PtdIns4P. Type II PIP kinases also produce PtdIns(4,5)P₂ using PtdIns5P as a substrate. Type III PIP kinases control production of PtdIns(3,5)P₂, a lipid that appears to have a role in membrane trafficking. Type I and II are further known to have a number of sub-types termed α, β, γ and splicing variants thereof (Tama, 1989 and references therein).

Although it has long been known that PIPkins are involved in the synthesis of PtdIns it is only recently that the mechanisms by which these pathways are controlled are successfully investigated.

Gozani et al, (2003), Cell 114: 99-111 reported that PtdIns5P binds to the PHD finger motif found in the putative tumour suppressor protein ING2 suggesting that this lipid is important in the response to DNA damage. It was also shown that the ING2 PHD finger interacts with PtdIns5P in vivo. This interaction regulates the ability of ING2 to activate p53 and p53-dependent apoptotic pathways. However, the authors do not provide any further information on how PtdIns5P or PtdIns4P level might be able to regulate an apoptotic response. WO03/105778 discloses compounds, compositions and methods for modulating the expression of phosphatidylinositol 4-phosphate 5-kinase type II beta for the treatment of various diseases involving angiogenesis including cancer. The documents characterises this enzyme as catalyzing the phosphorylation of PtdIns4P to PtdIns(4,5)P₂ however and indicates a correlation between the expression of the enzyme and cancer. The document does not investigate the role of PtdIns levels in diseases.

It has long been discussed that the phosphorylation of PtdIns3P to PtdIns(3,5)P₂ by phosphoinositide 3-kinase (PI-3-kinases) is involved in mitogenesis and transformation and might therefore be a valid drug target in the treatment of cancer (reviewed by Pendaries et al, (2003) FEBS Letters, 546: 25-31). For example, Prestwich (2004) Chemistry & Biology, 11: 619-637) discloses the use of phosphoinositides affinity probes for identifying lipid-protein interaction. The author discusses in particular the use of tethered PtdIns for drug discovery relating to cancer treatment regarding the type III PIP kinase signalling pathway. These documents are silent on the apoptotic pathway or the involvement of other PtdIns and respective kinases in said pathway.

### SUMMARY OF THE INVENTION

The present invention is based in part on the surprising finding that the PtdIns(4,5)P₂ level is instrumental in regulating apoptosis and cell out-growth, in particular it was found that cells have to maintain PtdIns(4,5)P₂ level in order to prevent apoptosis. Therefore the authors of the present invention postulate that by decreasing the levels of PtdIns(4,5)P₂, either by inhibiting the synthesis of PtdIns(4,5)P₂ or by systematically depleting the cell of PtdIns(4,5)P₂, apoptosis can be induced or may sensitise cells to apoptosis by other apoptotic agents such as used in cancer therapy.

The authors found also that the depletion of PtdIns(4,5)P₂ leads to neurite out growth, which indicates that PtdIns(4,5)P₂ may block said neurite growth, therefore the authors postulate the decrease of PtdIns(4,5)P₂ to stimulate neural outgrowth in diseases where neuronal outgrowth would be desirable. Alternatively, The authors postulate that by increasing PtdIns(4,5)P₂ apoptosis can be prevented.

Furthermore the authors of the present invention postulate that by increasing the levels of PtdIns5P or PtdIns4P for example by inhibiting the synthesis of PtdIns(4,5)P₂ by inhibiting type I or type II PIP kinase, apoptosis can be induced or may sensitise cells to apoptosis by other apoptotic agents such as used in cancer therapy.

An aspect of the invention is a method of modulating the PtdIns(4,5)P₂ level to modulate apoptosis. In particular one aspect of the invention is a method of combating a hyperproliferative disorder in an individual, comprising decreasing the levels of PtdIns(4,5)P₂ in the cell. A further aspect of the invention is a method to enhance neurite outgrowth and thus enhance nerve regeneration by decreasing the PtdIns(4,5)P₂ level. Another aspect of the invention is a method of combating diseases which would benefit from preventing apoptosis by increasing the PtdIns(4,5)P₂ level, for example cardiac reperfusion and some neuro-degenerative disorders such as Alzheimer's disease and Parkinson's disease. Another aspect of the invention is a method of combating a hyperproliferative disorder in an individual, comprising increasing the levels of PtdIns5P or PtdIns4P in the cell.

An aspect of the invention is a method of modulating apoptosis by modulating the levels of PtdIns(4,5)P₂ in the cell.

A further aspect of the invention is a method of inducing apoptosis by decreasing the levels of PtdIns(4,5)P₂ in the cell.

A further aspect of the invention is a method of sensitising a cell to apoptotic agents by decreasing the levels of PtdIns(4,5)P₂ in the cell.

A further aspect of the invention is a method as described above for treating a hyperproliferative disorder.

An preferred aspect of the invention is a method as described above whereby the hyperproliferative disorder is selected from: carcinoma, sarcoma, adenoma, hepatocellular carcinoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphangiosarcoma, synovioma, Ewing's tumor, leiomyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small lung carcinoma, bladder carcinoma, epithelial carcinoma, glioma, astrocytoma, medulloblastoma, craniopharyngioma, ependymoma, pinealoma, retinoblastoma multiple myeloma, rectal carcinoma, thyroid cancer, head and neck cancer, brain cancer, cancer of the peripheral nervous system, cancer of the central nervous system, neuroblastoma, cancer of the endometrium, myeloid lymphoma, leukemia, acute myelocytic leukemia, chronic leukemia, Hodgkin's lymphoma, non-Hodgkin's lymphoma and metastasis of all the above.

A further aspect of the invention is a method of stimulating neurite outgrowth by decreasing the levels of PtdIns(4,5)P₂ in the cell.

A preferred aspect of the invention is a method of stimulating neurite outgrowth by decreasing the levels of PtdIns(4,5)P₂ in the cell for treating a neurodegenerative disorder.

A further aspect of the invention is a method as described above whereby the neurodegenerative disorder is selected from: brain trauma, spinal cord trauma, trauma to the peripheral nervous system, Alzheimer's disease, Pick's disease, diffuse Lewy body disease, progressive supranuclear palsy (Steel-Richardson syndrome), multisystem degeneration (Shy-Drager syndrome), motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffman disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia, age-related dementia, vascular dementia, diffuse white matter disease (Binswanger's disease), dementia of endocrine or metabolic origin, dementia of head trauma and diffuse brain damage, dementia pugilistica or frontal lobe dementia, neurodegenerative disorders resulting from cerebral ischemia or infaction including embolic occlusion and thrombotic occlusion as well as intracranial hemorrhage of any type, intracranial and intravertebral lesions, hereditary cerebral angiopathy, normeuropathic hereditary amyloid, Down's syndrome, macroglobulinemia, secondary familial Mediterranean fever, Muckle-Wells syndrome, multiple myeloma, pancreatic- and cardiac-related amyloidosis, chronic hemodialysis arthropathy, or Finnish and Iowa amyloidosis.

In particular, diseases which are considered to benefit especially from this part of the invention (i.e. a method of combating a neurodegenerative disorder by promoting neurite outgrowth thus stimulating axonal regeneration, the method comprising decreasing the levels of PtdIns(4,5)P₂ in the cell) include: Brain trauma, spinal cord trauma, trauma to the peripheral nervous system, neurodegenerative disorders resulting from cerebral ischemia or infaction including embolic occlusion and thrombotic occlusion as well as intracranial hemorrhage of any type.

A further aspect of the invention is a method as described above whereby PtdIns(4,5)P₂ levels are decreased by inhibiting type I PIP kinase.

A further aspect of the invention is a method as described above whereby PtdIns(4,5)P₂ levels are decreased by inhibiting type II PIP kinase.

A further aspect of the invention is a method as described above whereby PtdIns(4,5)P₂ levels are decreased by inhibiting type I PIP kinase and type II PIP kinase.

A further aspect of the invention is a method as described above whereby levels of PtdIns(4,5)P₂ are decreased by contacting the cell with an agent.

A further aspect of the invention is a method as described above whereby the agent is an inhibitor of gene expression.

A further aspect of the invention is a method as described above wherein the agent acts via an RNA interference (RNAi) mechanism.

A further aspect of the invention is a method as described above whereby the agent acts via an antisense mechanism.

A further aspect of the invention is a method as described above—whereby the agent is a polynucleotide.

A further aspect of the invention is a method as described above—wherein the agent comprises a vector which is capable of expressing a polynucleotide in a host cell.

A further aspect of the invention is a method as described above wherein the vector is a viral vector.

A further aspect of the invention is a method as described above whereby the agent is polypeptide.

A further aspect of the invention is a method as described above whereby the agent is an enzyme inhibitor

A further aspect of the invention is a method as described above whereby the agent is a small molecule inhibitor.

A further aspect of the invention is a method as described above whereby the agent is an analogue of the substrate of type II PIP kinase or type I PIP kinase.

A further aspect of the invention is a use of an agent as described above in the manufacture of a medicament for use in combating a hyperproliferative disorder in an individual.

A further aspect of the invention is a use of an agent as described above in the manufacture of a medicament for use in combating a neurodegenerative disorder in an individual.

A further aspect of the invention is a pharmaceutical composition comprising an agent as defined above and a pharmaceutically acceptable carrier, diluent or excipient (including combinations thereof).

A further aspect of the invention is a method for identifying an agent that modulates (inhibits or activates) type I PIP kinase expression, the method comprising:
Providing a polynucleotide construct comprising a promoter of a gene encoding type I PIP kinase, or a functional equivalent thereof, operably linked to a coding sequence;
Providing a test agent;
Contacting a test substance with the polynucleotide construct under conditions that, in the absence of the test agent, would permit expression of the polypeptide encoded by the coding sequence;
Determining the level of expression of the polypeptide encoded by the coding sequence, thereby determining whether the test agent inhibits or activates the expression of the polypeptide.

In one aspect the method is for identifying an agent that inhibits type I PIP kinase expression.

A further aspect of the invention is a method for identifying an agent that modulates (inhibits or activates) type II PIP kinase expression, the method comprising:
Providing a polynucleotide construct comprising a promoter of a gene encoding type II PIP kinase, or a functional equivalent thereof, operably linked to a coding sequence;
Providing a test agent;
Contacting a test substance with the polynucleotide construct under conditions that, in the absence of the test agent, would permit expression of the polypeptide encoded by the coding sequence;
Determining the level of expression of the polypeptide encoded by the coding sequence, thereby determining whether the test agent inhibits or activates the expression of the polypeptide.

In one aspect the method is for identifying an agent that inhibits type II PIP kinase expression.

A further aspect of the invention is a method for identifying an agent that modulates (inhibits or activates) type I PIP kinase activity, the method comprising:
Contacting a test agent with an assay composition under conditions that, in the absence of the test substance, would allow type I PIP kinase activity.
Determining the level of activity of the type I PIP kinase thereby to determine whether the test agent inhibits or activates the activity of type I PIP kinase.

A further aspect of the invention is the method as described above whereby the assay composition comprises a reaction mixture of:
A fluorescently labelled product or product analogue of type I PIP kinase bound to phospholipase C or a fragment thereof;
type I PIP kinase, or a fragment thereof; and
an unlabelled substrate of type I PIP kinase or unlabelled substrate analogue thereof.

The effect of an agent on the kinase activity can be measured by comparing the rate of the signal decrease in the presence of said agent with the rate of the signal decrease in the absence of said agent.

A further aspect of the invention is the method as described above whereby fluorescently labelled product analogue is selected from the group of PtdIns(3,5)P₂, PtdIns(4,5)P₂ and PtdIns(3,4,5)P₃

A further aspect of the invention is the method for identifying an agent that modulates (inhibits or activates) type II PIP kinase activity, the method comprising:
Contacting a test agent with an assay composition under conditions that, in the absence of the test substance, would allow type II PIP kinase activity;
Determining the level of activity of the type II PIP kinase thereby to determine whether the test agent inhibits or activates the activity of type II PIP kinase.

A further aspect of the invention is the method as described above whereby the assay composition comprises a reaction mixture of:
A fluorescently labelled product or product analogue of type II PIP kinase bound to phospholipase C or a fragment thereof;
type II PIP kinase, or a fragment thereof; and
an unlabelled substrate of type II PIP kinase or unlabelled substrate analogue thereof.

The effect of an agent on the kinase activity can be measured by comparing the rate of the signal decrease in the presence of said agent with the rate of the signal decrease in the absence of said agent.

A further aspect of the invention is the method as described above whereby fluorescently labelled product analogue is selected from the group of PtdIns(3,4)P₂, PtdIns(4,5)P₂ and PtdIns(3,4,5)P₃

Typically, a decrease in the activity of type I PIP kinase or type II PIP kinase indicates that the agent may be an inhibitor of the type I PIP kinase or type II PIP kinase, whereas an increase in the activity of type I PIP kinase or type II PIP kinase indicates that the agent may be an activator of the type I PIP kinase or type II PIP kinase.

A further aspect of the invention is a method as defined above whereby the agent identified is modified to improve its activity and pharmaceutical properties and retested.

A further aspect of the invention is method for identifying an agent as described above which further comprises:
Contacting the agent identified with a mammalian cell;
Determining the efficacy of the agent for a hyperproliferative or neurodegenerative disorder.

A further aspect of the invention is a method for identifying an agent as described above which further comprises:
Testing the agent identified in an animal model;
Determining the efficacy of the agent for a hyperproliferative or neurodegenerative disorder.

A further aspect of the invention is a method for identifying an agent as described above which further comprises:
Testing the agent identified in a clinical trial for safety
Determining the efficacy of the agent for a hyperproliferative or neurodegenerative disorder.

In one aspect the invention relates to a method for identifying an agent for modulating apoptosis or neurite outgrowth in cells, or for treating a hyperproliferative disorder or a neurodegenerative disease, wherein the method is any of the identification methods described herein.

A further aspect of the invention is a method for the preparation of a pharmaceutical composition, comprising identifying an agent by a method as defined above and formulating the agent with a pharmaceutically acceptable carrier thereof.

A further aspect of the invention is an agent identified by a method as defined above.

A further aspect of the invention is a pharmaceutical composition comprising an agent as defined above, for example, an agent identified by a method described herein, and a pharmaceutically acceptable carrier, diluent or excipient (including combinations thereof).

A further aspect of the invention is the use of an agent identified by a method as defined above for the preparation of a medicament for the treatment of a hyperproliferative or neurodegenerative disease.

A further aspect of the invention is a method of preventing apoptosis by increasing the levels of PtdIns(4,5)P₂ in the cell.

A further aspect of the invention (i.e. the method of preventing apoptosis by increasing the levels of PtdIns(4,5)P₂ in the cell) is the treatment of diseases such as cardiac reperfusion or neurodegenerative diseases (arresting the neurodegenerative disease by preventing apoptosis).

A further aspect of the invention is a method as described above whereby PtdIns(4,5)P₂ levels are increased by stimulating or inducing type I PIP kinase.

A further aspect of the invention is a method as described above whereby PtdIns(4,5)P₂ levels are increased by stimulating or inducing type II PIP kinase.

A further aspect of the invention is a method as described above whereby PtdIns(4,5)P₂ levels are increased by stimulating or inducing type I PIP kinase and type II PIP kinase.

A further aspect of the invention is a method as described above whereby levels of PtdIns(4,5)P₂ are increased by contacting the cell with an agent.

A further aspect of the invention is a method as described above whereby the agent is an activator of gene expression.

A further aspect of the invention is a method as described above—whereby the agent is a polynucleotide.

A further aspect of the invention is a method as described above—wherein the agent comprises a vector which is capable of expressing a polynucleotide in a host cell.

A further aspect of the invention is a method as described above wherein the vector is a viral vector.

A further aspect of the invention is a method as described above whereby the agent is polypeptide.

A further aspect of the invention is a method as described above whereby the agent is an enzyme activator.

A further aspect of the invention is a method as described above whereby the agent is a small molecule activator.

A further aspect of the invention is a method of preventing apoptosis by increasing the levels of PtdIns(4,5)P₂ in the cell as described above whereby the agent is an analogue of the substrate of type II PIP kinase or type I PIP kinase.

Agents and pharmaceutical compositions suitable for the invention (i.e. method of preventing apoptosis by increasing the levels of PtdIns(4,5)P₂ in the cell as described above), can be identifying by methods described above.

A further aspect of the invention is a method of inducing apoptosis by increasing the levels of PtdIns5P or PtdIns4P in the cell.

A further aspect of the invention is a method of sensitising a cell to apoptotic agents by increasing the levels of PtdIns5P or PtdIns4P in the cell.

A further aspect of the invention is a method for treating a hyperproliferative disorder by increasing the levels of PtdIns5P or PtdIns4P in the cell. Hyperproliferative disorders that might be treated are listed above.

A further aspect of the invention is a method as described above whereby PtdIns5P or PtdIns4P levels are increased by inhibiting type II PIP kinase or type I PIP kinase respectively. Methods of achieving inhibition of type II or type I PIP kinase, agents inhibiting type II or type I PIP kinase and methods of identifying of such agents are described above.

The invention also relates to an agent that inhibits type I PIP kinase and/or type II PIP kinase expression and/or activity for use in:
(i) inducing apoptosis or sensitising cells to apoptotic agents;
(ii) stimulating neurite outgrowth;
(iii) treating hyperproliferative disorders or neurodegenerative disease.

The invention further relates to the use of an agent that inhibits type I PIP kinase and/or type II PIP kinase expression and/or activity for the manufacture of a medicament for:
(i) inducing apoptosis or sensitising cells to apoptotic agents;
(ii) stimulating neurite outgrowth;
(iii) treating hyperproliferative disorders or neurodegenerative disease.

The invention also relates to an agent that enhances type I PIP kinase and/or type II PIP kinase expression and/or activity for use in:
(i) preventing apoptosis;
(ii) treating cardiac reperfusion or neurodegenerative disease.

The invention further relates to use of an agent that enhances type I PIP kinase and/or type II PIP kinase expression and/or activity for the manufacture of a medicament for:
(i) preventing apoptosis;
(ii) treating cardiac reperfusion or neurodegenerative disease.

In one aspect the inhibiting or enhancing agent has been identified by a method described herein.

### DETAILED DESCRIPTION

By “decreasing the levels of PtdIns(4,5)P₂” we include the meaning of decreasing the concentration of phosphatidylinositol(4,5)bisphosphate. The decrease can be a low level decrease of about 10%, or about 20%, or about 30%, or about 40% of the concentration of PtdIns(4,5)P₂. The decrease can be a medium level decrease of about 50%, or about 60%, or about 70%, or about 80% decrease of the concentration of PtdIns(4,5)P₂. The decrease can also be a high level decrease of about 90%, or about 95%, or about 99%, or about 99.9%, or about 99.99% of the concentration of PtdIns(4,5)P₂.

By “increasing the levels of PtdIns(4,5)P₂” we include the meaning of increasing the concentration of phosphatidylinositol (4,5)-bisphosphate. The increase can be a low level increase of about 10%, or about 20%, or about 30%, or about 40% of the concentration of PtdIns(4,5)P₂. The increase can be a medium level increase of about 50%, or about 60%, or about 70%, or about 80% increase of the concentration of PtdIns(4,5)P₂. The increase can also be a high level increase of about 90%, or about 95%, or about 99%, or about 99.9%, or about 99.99% of the concentration of PtdIns(4,5)P₂.

By “apoptosis” we mean the process of programmed cell death which is characterized by morphologic nuclear (nuclear blebbing to form micronuclei, condensation of chromatin and DNA fragmentation) and physiological (loss of intercellular contact, and vacuolation with a relative conservation of cellular organelles) changes to the cell.

Methods for determining apoptosis in cells are known to those skilled in the art. For example, apoptotic nuclear changes may be determined by staining with 160 mg/ml bisbenzimide Hoechst 33258 and counting down a microscope.

Thus, modulating apoptosis in cells may refer to increasing or decreasing the rate of apoptosis, in cells. Modulation may include, for example, inducing apoptosis in cells that would not enter programmed cell death in the absence of the inducing agent. It may include increasing the proportion of cells in apoptosis in a cell population. Sensitising cells to apoptotic agents may refer to increasing the rate at which cells die in response to an apoptotic agent, and/or increasing the proportion of cells in a population that enter apoptosis in response to an apoptotic agent.

Preventing apoptosis may refer to reducing the rate of cell death in a cell population. This may include reducing the proportion of cells that enter apoptosis in a cell population. This may also include reducing the sensitivity of cells to apoptotic agents.

Typically an increase or decrease in apoptosis refers to a statistically significant increase or decrease in the number of cells, following staining with Hoechst 33258, displaying apoptotic nuclear changes when observed under a fluorescent microscope.

Apoptotic agents typically comprise substances that are capable of inducing apoptosis in cells. Examples of such agents are known in the art and include H₂O₂ and UV radiation.

By “type I PIP kinase” we include the gene product of the human type I PIP kinase, preferably type I α PIP kinase, gene and naturally occurring variants thereof. The sequence of the human type I PIP kinase gene is found in Genbank Accession No. NM_{—}003557 and the human type I PIP kinase cDNA sequence is listed in FIG. 18 Human type I PIP kinase includes the amino acid sequence listed in FIG. 18 and naturally occurring variants thereof. As above, type I PIP kinase includes sub-types termed α, β, γ and splicing variants thereof.

By “type I PIP kinase” we also include a homologous gene product from type I PIP kinase genes from other species, including the mouse. The cDNA and amino acid sequence of mouse type I PIP kinase includes the sequences listed in FIG. 17 Genbank Accession No. NM_{—}008846

By “type II PIP kinase” we include the gene product of the human type II PIP kinase, preferably type II β PIP kinase gene and naturally occurring variants thereof. The sequence of the human type II PIP kinase gene is found in Genbank Accession No. HSU85245, and the human type II PIP kinase cDNA sequence is listed in FIG. 16. Human type II PIP kinase includes the amino acid sequence listed in FIG. 16 and naturally occurring variants thereof. As above, type II PIP kinase includes sub-types termed α, β, γ and splicing variants thereof.

By “type II PIP kinase” we also include a homologous gene product from type II PIP kinase genes from other species, including type II PIP kinase from the rat. The cDNA and amino acid sequence of rat type II PIP kinase includes the sequences listed in FIG. 15 Genbank Accession No. AF033355.

By “homologous gene product” we include a type I PIP kinase or type II PIP kinase polypeptide having at least 80% sequence identity with the human type I PIP kinase or type II PIP kinase amino acid sequence in FIG. 18 or FIG. 16. More preferably, a homologous gene product includes a type I PIP kinase or type II PIP kinase polypeptide having at least 84% sequence identity with human type I PIP kinase or type II PIP kinase. Yet more preferably, a homologous gene product includes a type I PIP kinase or type II PIP kinase polypeptide having at least 91%, or at least 92%, or at least 93%, or at least 94%, or at least 95%, or at least 96%, or at least 97%, or at least 98% sequence identity with human type I PIP kinase or type II PIP kinase. Most preferably, a homologous gene product includes a type I PIP kinase or type II PIP kinase polypeptide having at least 99% sequence identity with the human type I PIP kinase or type II PIP kinase amino acid sequence.

By inhibiting type I PIP kinase or type II PIP kinase we mean inhibiting type I PIP kinase or type II PIP kinase activity or expression of the type I PIP kinase or type II PIP kinase gene product.

Activating, stimulating or inducing type I PIP kinase or type II PIP kinase typically comprises increasing or enhancing type I PIP kinase or type II PIP kinase activity or expression of the type I PIP kinase or type II PIP kinase gene product.

By a “type I PIP kinase activity” or a “type II PIP kinase activity” we include the meaning of any activity, function or interaction of type I PIP kinase or type II PIP kinase, or any process performed on, by, or involving type I PIP kinase or type II PIP kinase, that occurs within a cell. Thus type I PIP kinase activity includes, but is not limited to, the phosphorylation of: PtdIns4P, PtdIns(3,4)P₂, PtdIns3P and PtdIns, wherein PtdIns4P is the preferred substrate.

Type II PIP kinase (for example, type II β PIP kinase) activity includes, but is not limited to, the phosphorylation of: PtdIns3P, PtdIns(3,5)P₂, PtdIns and PtdIns5P, wherein PtdIns5P is the preferred substrate.

By “inhibiting type I PIP kinase or type II PIP kinase activity” we include the meaning of reducing the rate or level of an activity of type I PIP kinase or type II PIP kinase. The reduction can be a low level reduction of about 10%, or about 20%, or about 30%, or about 40% of an activity of type I PIP kinase or type II PIP kinase. The reduction can be a medium level reduction of about 50%, or about 60%, or about 70%, or about 80% reduction of an activity of type I PIP kinase or type II PIP kinase. The reduction can also be a high level reduction of about 90%, or about 95%, or about 99%, or about 99.9%, or about 99.99% of an activity of type I PIP kinase or type II PIP kinase. Inhibition can also include the elimination of an activity of type I PIP kinase or type II PIP kinase or its reduction to an undetectable level.

By “inhibiting expression of type I PIP kinase or type II PIP kinase” we include the meaning of reducing the expression of the type I PIP kinase or type II PIP kinase gene product. The reduction can be a low level reduction of about 10%, or about 20%, or about 30%, or about 40% of the expression of type I PIP kinase or type II PIP kinase. The reduction can be a medium level reduction of about 50%, or about 60%, or about 70%, or about 80% reduction of the expression of type I PIP kinase or type II PIP kinase. The reduction can also be a high level reduction of about 90%, or about 95%, or about 99%, or about 99.9%, or about 99.99% of an activity of the expression of type I PIP kinase or type II PIP kinase. Inhibition can also include the elimination of the expression of type I PIP kinase or type II PIP kinase or its reduction to an undetectable level.

“Enhancing type I PIP kinase or type II PIP kinase activity” typically comprises increasing the rate or level of an activity of type I PIP kinase or type II PIP kinase. The increase can be a low level increase of about 10%, or about 20%, or about 30%, or about 40% of an activity of type I PIP kinase or type II PIP kinase. The increase can be a medium level increase of about 50%, or about 60%, or about 70%, or about 80% increase of an activity of type I PIP kinase or type II PIP kinase. The increase can also be a high level increase of about 90%, or about 95%, or about 99%, or about 99.9%, or about 99.99% or more of an activity of type I PIP kinase or type II PIP kinase. There may also be an increase of an activity of type I PIP kinase or type II PIP kinase of higher level, for example, 100%, 150%, 200% or more.

“Enhancing expression of type I PIP kinase or type II PIP kinase” typically comprises increasing the expression of the type I PIP kinase or type II PIP kinase gene product. The increase can be a low level increase of about 10%, or about 20%, or about 30%, or about 40% of the expression of type I PIP kinase or type II PIP kinase. The increase can be a medium level increase of about 50%, or about 60%, or about 70%, or about 80% increase of the expression of type I PIP kinase or type II PIP kinase. The increase can also be a high level increase of about 90%, or about 95%, or about 99%, or about 99.9%, or about 99.99% of an activity of the expression of type I PIP kinase or type II PIP kinase. There may also be an increase at a higher level, for example, 100%, 150%, 200% or more.

By “neurite” we mean any immature neuronal process (axon or dendrite). By “promoting neurite outgrowth” or “stimulating neurite outgrowth” we mean increasing the numbers of new neurite outgrowths from the cell. By “stimulating axonal regeneration” we mean the increasing the growth of cellular extensions that project to, and make synapses with, the dendrites of other neurons. This may be determined by manual examination of cells under a microscope.

An agent which decreases the levels of PtdIns(4,5)P₂ may be an inhibitor of type I PIP kinase or type II PIP kinase activity which is an inhibitor of the protein or it may be an inhibitor of the production of the type I PIP kinase or type II PIP kinase protein from its gene. It is preferred if the inhibitor is selective for type I PIP kinase or type II PIP kinase. By selective we mean that the inhibitor has a greater affinity for of type I PIP kinase or type II PIP kinase (or its gene or mRNA) than for other proteins or genes or mRNA within the cell. It will be appreciated that it is not necessary for the inhibitor to be completely specific for of type I PIP kinase or type II PIP kinase; rather, it is acceptable for the inhibitor to display a degree of selectivity for of type I PIP kinase or type II PIP kinase even though it may also be active against other components of the cell.

It is particularly preferred if the inhibitor is selective for type I PIP kinase or type II PIP kinase compared to other members of the PIP kinase family.

Examples of inhibitors of type I PIP kinase or type II PIP kinase include duplex RNA which mediates interference of type I PIP kinase or type II PIP kinase RNA, antisense nucleic acid, ribozymes selective for type I PIP kinase or type II PIP kinase mRNA, antibodies which bind to type I PIP kinase or type II PIP kinase, and small molecules which inhibit its function.

Agents that inhibit type I PIP kinase or type II PIP kinase gene expression include antisense RNA, small interfering RNAs (such as described in Hannon et al. Nature, 418 (6894): 244-51 (2002); Brummelkamp et al., Science 21, 21 (2002); and Sui et al., Proc. Natl. Acad. Sci. USA 99, 5515-5520 (2002), and described below), and ribozyme molecules which selectively cleave polynucleotides encoding type I PIP kinase or type II PIP kinase.

Agents that inhibit type I PIP kinase or type II PIP kinase gene transcription can be designed, for example using an engineered transcription repressor described in Isalan et al. Nat Biotechnol, 19(7): 656-60 (2001) and in Urnov F. Biochem Pharmacol, 64 (5-6):919 (2002), or they can be selected, for example using the screening methods described in later aspects of the invention.

RNA interference (RNAi) is the process of sequence-specific post-transcriptional gene silencing in animals initiated by double-stranded (dsRNA) that is homologous in sequence to the silenced gene. The mediators of sequence-specific mRNA degradation are typically 21- and 22-nucleotide small interfering RNAs (siRNAs) which, in vivo, may be generated by ribonuclease III cleavage from longer dsRNAs. Elbashir et al. (2001, Nature 411, 494-498) has shown that 21-nucleotide siRNA duplexes specifically suppress expression of both endogenous and heterologous genes in, for example, mammalian cells. In mammalian cells it is believed that the siRNA has to be comprised of two complementary 21mers as described below since longer double-stranded (ds) RNAs will activate PKR (dsRNA-dependent protein kinase) and inhibit overall protein synthesis.

Duplex siRNA molecules selective for type I PIP kinase or type II PIP kinase can readily be designed by reference to the type I PIP kinase or type II PIP kinase cDNA sequence. For example, they can be designed by reference to the human type I PIP kinase or type II PIP kinase cDNA sequence shown in FIGS. 18 and 16, or naturally occurring variants thereof. Typically, the first 21-mer sequence that begins with an AA dinucleotide which is at least 120 nucleotides downstream from the initiator methionine codon is selected. The RNA sequence perfectly complementary to this becomes the first RNA oligonucleotide. The second RNA sequence should be perfectly complementary to the first 19 residues of the first, with an additional UU dinucleotide at its 3′ end. Once designed, the synthetic RNA molecules can be synthesised using methods well known in the art.

siRNAs may be introduced into cells in the patient using any suitable method. Typically, the RNA is protected from the extracellular environment, for example by being contained within a suitable carrier or vehicle. Liposome-mediated transfer is preferred. Liposomes are described in more detail with respect to antisense nucleic acids below. It is particularly preferred if the oligofectamine method is used.

Antisense nucleic acid molecules selective for type I PIP kinase or type II PIP kinase can be designed by reference to the cDNA or gene sequence, as is known in the art.

Antisense nucleic acids, such as oligonucleotides, are single-stranded nucleic acids, which can specifically bind to a complementary nucleic acid sequence. By binding to the appropriate target sequence, an RNA-RNA, a DNA-DNA, or RNA-DNA duplex is formed. These nucleic acids are often termed “antisense” because they are complementary to the sense or coding strand of the gene. Recently, formation of a triple helix has proven possible where the oligonucleotide is bound to a DNA duplex. It was found that oligonucleotides could recognise sequences in the major groove of the DNA double helix. A triple helix was formed thereby. This suggests that it is possible to synthesise a sequence-specific molecules which specifically bind double-stranded DNA via recognition of major groove hydrogen binding sites.

By binding to the target nucleic acid, the above oligonucleotides can inhibit the function of the target nucleic acid. This could, for example, be a result of blocking the transcription, processing, poly(A)addition, replication, translation, or promoting inhibitory mechanisms of the cells, such as promoting RNA degradations.

Antisense oligonucleotides are prepared in the laboratory and then introduced into cells, for example by microinjection or uptake from the cell culture medium into the cells, or they are expressed in cells after transfection with plasmids or retroviruses or other vectors carrying an antisense gene. Antisense oligonucleotides were first discovered to inhibit viral replication or expression in cell culture for Rous sarcoma virus, vesicular stomatitis virus, herpes simplex virus type 1, simian virus and influenza virus. Since then, inhibition of mRNA translation by antisense oligonucleotides has been studied extensively in cell-free systems including rabbit reticulocyte lysates and wheat germ extracts. Inhibition of viral function by antisense oligonucleotides has been demonstrated in vitro using oligonucleotides which were complementary to the AIDS HIV retrovirus RNA (Goodchild, J. 1988 “Inhibition of Human Immunodeficiency Virus Replication by Antisense Oligodeoxynucleotides”, Proc. Natl. Acad. Sci. (USA) 85(15), 5507-11). The Goodchild study showed that oligonucleotides that were most effective were complementary to the poly(A) signal; also effective were those targeted at the 5′ end of the RNA, particularly the cap and 5′ untranslated region, next to the primer binding site and at the primer binding site. The cap, 5′ untranslated region, and poly(A) signal lie within the sequence repeated at the ends of retrovirus RNA (R region) and the oligonucleotides complementary to these may bind twice to the RNA.

Typically, antisense oligonucleotides are 15 to 35 bases in length. For example, 20-mer oligonucleotides have been shown to inhibit the expression of the epidermal growth factor receptor mRNA (Witters et al., Breast Cancer Res Treat 53:41-50 (1999)) and 25-mer oligonucleotides have been shown to decrease the expression of adrenocorticotropic hormone by greater than 90% (Frankel et al., J Neurosurg 91:261-7 (1999)). However, it is appreciated that it may be desirable to use oligonucleotides with lengths outside this range, for example 10, 11, 12, 13, or 14 bases, or 36, 37, 38, 39 or 40 bases.

Antisense polynucleotides may be administered systemically. Alternatively the inherent binding specificity of polynucleotides characteristic of base pairing is enhanced by limiting the availability of the polynucleotide to its intended locus in vivo, permitting lower dosages to be used and minimising systemic effects. Thus, polynucleotides may be applied locally to achieve the desired effect. The concentration of the polynucleotides at the desired locus is much higher than if the polynucleotides were administered systemically, and the therapeutic effect can be achieved using a significantly lower total amount. The local high concentration of polynucleotides enhances penetration of the targeted cells and effectively blocks translation of the target nucleic acid sequences.

It will be appreciated that antisense agents also include larger molecules which bind to type I PIP kinase or type II PIP kinase mRNA or genes and substantially prevent expression of type I PIP kinase or type II PIP kinase mRNA or genes and substantially prevent expression of the type I PIP kinase or type II PIP kinase. Thus, an antisense molecule which is substantially complementary to type I PIP kinase or type II PIP kinase mRNA is envisaged as part of the invention.

The larger molecules may be expressed from any suitable genetic construct and delivered to the patient. Typically, the genetic construct which expresses the antisense molecule comprises at least a portion of the type I PIP kinase or type II PIP kinase cDNA or gene operatively linked to a promoter which can express the antisense molecule in the cell.

Although genetic constructs for delivery of polynucleotides can be DNA or RNA it is preferred if it is DNA. Equivalent genetic constructs can be used to deliver antisense polynucleotides to a patient as described herein in relation to the delivery of polynucleotides encoding type I PIP kinase or type II PIP kinase.

Preferably, the genetic construct is adapted for delivery to a human cell.

In a preferred embodiment, the polynucleotide which is antisense further comprises a vector which is designed to express antisense DNA. Hence, the invention further provides a polynucleotide comprising a nucleic acid sequence which is antisense to a polynucleotide encoding the type I PIP kinase or type II PIP kinase polypeptide for use in medicine, especially in the manufacture of a medicament for treating hyperproliferative and neurodegenerative disorders.

Ribozymes are RNA or RNA-protein complexes that cleave nucleic acids in a site-specific fashion. Ribozymes have specific catalytic domains that possess endonuclease activity. For example, a large number of ribozymes accelerate phosphoester transfer reactions with a high degree of specificity, often cleaving only one of several phosphoesters in an oligonucleotide substrate. This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids. For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications, and may be designed by reference to the cDNA which is a copy of the mRNA to be cleaved (e.g. the human type I PIP kinase or type II PIP kinase cDNA shown in FIGS. 18 and 16, or naturally occurring variants thereof).

The invention also includes variants of the polynucleotides encoding the agents described above, or variants of the antisense polynucleotides, or variants of the siRNAs.

The term “variant” includes polynucleotides having at least 90%, preferably at least 91%, or at least 92%, or more preferably at least 93%, or at least 94%, or at least 95%, or at least 96%, or yet more preferably at least 97%, or at least 98%, or most preferably at least 99% sequence identity with the polynucleotides encoding the agents described above, or the antisense polynucleotides, or the siRNAs.

The term “variant” also encompasses sequences that are complementary to sequences that are capable of hybridising under highly stringent conditions (e.g. 65° C. and 0.1×SSC {1×SSC=0.15 M NaCl, 0.015 M Na3 citrate pH 7.0}) to polynucleotides encoding the agents described above, or to the polynucleotide agents.

As used herein, reference to a polypeptide comprising a specific fragment, domain, region or sequence of a protein does not include the full-length protein sequence. The polypeptide can comprise the specific fragment, domain, region or sequence and at least 1, or at least 2, or at least 5, or at least 10, or at least 20, or at least 50, or at least 100, or at least 200 or more amino acids from the full-length protein C and/or N terminal or the specific fragment, domain, region or sequence, providing that the polypeptide does not comprise the full-length protein. Thus, for example, the polypeptide can comprise a deletion mutant of the full-length protein.

Alternatively, the polypeptide can comprise the specific fragment, domain, region or sequence and exogenous C and/or N terminal amino acid sequences of any length.

By “exogenous” we include the meaning that the C and/or N terminal amino acid sequences are not found in the full-length protein.

By an agent “selectively binding” a specified domain of a target protein, we include the meaning that the agent binds the specific domain with a greater affinity than for any other region of the target protein. Preferably, the agent binds the specific domain with at least 2, or at least 5, or at least 10 or at least 50 times greater affinity than any other region of the target protein. More preferably, the agent binds the specific domain with at least 100, or at least 1,000, or at least 10,000 times greater affinity than any other region of the target protein.

Preferably, when the target protein is present in a cell, the agent binds the target protein at the specific domain with a greater affinity than for any other molecule in the cell. Preferably, the agent binds the target protein at the specific domain with at least 2, or at least 5, or at least 10 or at least 50 times greater affinity than for any other molecule in the cell. More preferably, the agent binds the target protein at the specific domain with at least 100, or at least 1,000, or at least 10,000 times greater affinity than any other molecule in the cell.

The term “antibody” as used herein includes but is not limited to polyclonal, monoclonal, chimaeric, single chain, Fab fragments and fragments produced by a Fab expression library. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. Furthermore, the antibodies and fragments thereof may be humanised antibodies, for example as described in U.S. Pat. No. 239,400. Neutralising antibodies, ie, those which inhibit biological activity of the substance polypeptides, are especially preferred for diagnostics and therapeutics.

Antibodies may be produced by standard techniques, for example by immunisation with the appropriate fragment of type I PIP kinase or type II PIP kinase, or by using a phage display library.

If polyclonal antibodies are desired, a selected mammal (e.g. mouse, rabbit, goat, horse, etc.) is immunised with an immunogenic polypeptide bearing a epitope such as the particular type I PIP kinase or type II PIP kinase domains described herein. Depending on the host species, various adjuvants may be used to increase immunological response. Such adjuvants include, but are not limited to, Freund', mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to an epitope obtainable from an identified agent and/or substance of the present invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against particular epitopes, such as the particular domains or fragments of type I PIP kinase or type II PIP kinase described herein, can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; ie, for isotype and epitope affinity.

Monoclonal antibodies may be prepared using any technique which provides for the production of antibody molecules by continuous cell lines in culture. These include, but are not limited to, the hybridoma technique originally described by Koehler and Milstein (1975 Nature 256: 495-497), the human B-cell hybridoma technique (Kosbor et al. (1983) Immunol Today 4: 72; Cote et al. (1983) Proc Natl Acad Sci 80: 2026-2030) and the EBV-hybridoma technique (Cole et al. (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al. (1984) Proc Natl Acad Sci 81: 6851-6855; Neuberger et al. (1984) Nature 312: 604-608; Takeda et al. (1985) Nature 314: 452-454). Alternatively, techniques described for the production of single chain antibodies (U.S. Pat. No. 4,946,779) can be adapted to produce the single chain antibodies specific to, for example, a particular type I PIP kinase or type II PIP kinase domain.

Antibodies may also be produced by inducing in vivo production in the lymphocyte population or by screening recombinant immunoglobulin libraries or panels of highly specific binding reagents as disclosed in Orlandi et al. (1989, Proc Natl Acad Sci 86: 3833-3837), and Winter G and Milstein C (1991; Nature 349: 293-299).

Antibody fragments which contain specific binding sites for the substance may also be generated. For example, such fragments include, but are not limited to, the F(ab′)2 fragments which can be produced by pepsin digestion of the antibody molecule and the Fab fragments which can be generated by reducing the disulphide bridges of the F(ab′)2 fragments. Alternatively, Fab expression libraries may be constructed to allow rapid and easy identification of monoclonal Fab fragments with the desired specificity (Huse W D et al. (1989) Science 256: 1275-1281).

An agent which increases the levels of PtdIns(4,5)P₂ in the cell typically increases or enhances expression and/or activity of type I or type II PIP kinase as defined herein. An agent may stimulate or activate expression or activity. An agent may comprise a polynucleotide or a polypeptide. For example, an agent may comprise a vector, e.g. a viral vector, which is capable of expressing a polynucleotide encoding type I or type II PIP kinase in a cell.

An agent may comprise an activator of type I or type II PIP kinase, for example a small molecule activator. An agent may comprise, for example, an analogue of a substrate of type I PIP kinase, or type II PIP kinase, as described herein. Agents which act to positively modulate type I or type II PIP kinase may be identified by the methods described herein.

The invention also includes polynucleotides encoding the polypeptide agents or the nucleic acid agents described herein, for example those that modulate type I PIP kinase or type II PIP kinase gene expression.

A polynucleotide encoding a polypeptide agent, for example a single chain antibody that binds to a specific region of type I PIP kinase or type II PIP kinase, or a type I PIP kinase or type II PIP kinase fragment or mutant fragment or mutant, may be administered to a target cell as described herein. Expression of the agent from the polynucleotide thus results in intra-cellular administration of the polypeptide agent. Similarly, a polynucleotide encoding a nucleic acid agent, for example an antisense agent that modulates type I PIP kinase or type II PIP kinase gene expression, can be administered intra-cellularly. Depending on the mode of administration, the polynucleotide can be administered into the nucleus or the cytoplasm of the target cell as desired. Suitable vectors include both viral and non-viral vectors, such as those described herein, and are well known to a person of skill in the art

Typically, a polynucleotide encoding an agent is operably linked to a regulatory sequence which is capable of providing for the expression of the polynucleotide, in or by a chosen host cell. The invention includes a genetic construct, such as a vector, comprising the polynucleotide of the present invention operably linked to such a regulatory sequence.

The term “operably linked” refers to a juxtaposition wherein the components described are in a relationship permitting them to function in their intended manner e.g. to express the polypeptide agent. The term “regulatory sequences” includes promoters and enhancers and other expression regulation signals. The term “promoter” is used in the normal sense of the art, e.g. an RNA polymerase binding site.

As used herein, the term “nucleotide sequence” is synonymous with the term “polynucleotide” and “nucleic acid”. The nucleotide sequence may be DNA or RNA of genomic or synthetic or of recombinant origin. The nucleotide sequence may be double-stranded or single-stranded whether representing the sense or antisense strand or combinations thereof.

For some applications, preferably, the nucleotide sequence is DNA. For some applications, preferably, the nucleotide sequence is prepared by use of recombinant DNA techniques (e.g. recombinant DNA). For some applications, preferably, the nucleotide sequence is cDNA. For some applications, preferably, the nucleotide sequence may be the same as the naturally occurring form.

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4th Ed, John Wiley & Sons, Inc. PCR is described in U.S. Pat. Nos. 4,683,195, 4,800,195 and 4,965,188.

By “analogue of PtdIns4P”, we mean a molecule which will have structural similarities to PtdIns4P enabling it to compete with PtdIns4P in binding to a receptor of PtdIns4P. Preferred analogues are analogues based on inositol(1,4)bis phosphate which lack the fatty acid chain.

Similarly an “analogue of PtdIns5P” typically comprises a molecule which will have structural similarities to PtdIns5P enabling it to compete with PtdIns5P in binding to a receptor of PtdIns5P.

Similarly an “analogue of PtdIns(4,5)P₂ typically comprises a molecule which will have structural similarities to PtdIns(4,5)P₂ enabling it to compete with PtdIns(4,5)P₂ in binding to a receptor of PtdIns(4,5)P₂.

By “combating” we include the meaning that the method can be used to alleviate symptoms of the disorder (ie the method is used palliatively), or to treat the disorder, or to prevent the disorder (ie the method is used prophylactically).

The method of combating a disorder may applied to humans or animals. Preferably, the methods of the inventions are used to combat a disorder in humans.

An aspect of the invention provides the use of an agent which inhibits type I PIP kinase or type II PIP kinase activity in the manufacture of a medicament for combating a disorder that would benefit from inhibition of type I PIP kinase or type II PIP kinase activity. For example, the disorder may be a hyperproliferative disorder such as those described herein, or a neurodegenerative disorder such as those described herein.

An aspect of the invention is a pharmaceutical composition comprising an agent which decreases the level of PtdIns(4,5)P₂ within a cell and a pharmaceutically acceptable carrier, diluent or excipient.

A further aspect of the present invention provides a pharmaceutical composition comprising an agent which inhibits a type I PIP kinase or type II PIP kinase activity and a pharmaceutically acceptable carrier, diluent or excipient (including combinations thereof).

The invention includes a pharmaceutical composition comprising a polynucleotide that encodes an agent which inhibits type I PIP kinase or type II PIP kinase and a pharmaceutically acceptable carrier, diluent or excipient (including combinations thereof).

Preferably and typically, agents that inhibit type I PIP kinase or type II PIP kinase are as described above and herein.

The pharmaceutical compositions may be for human or veterinary medicine and will typically comprise any one or more of a pharmaceutically acceptable diluent, carrier, or excipient.

Acceptable carriers or diluents for therapeutic use are well known in the pharmaceutical art, and are described, for example, in Remington' Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder, lubricant, suspending agent, coating agent, solubilising agent.

Preservatives, stabilizers, dyes and even flavouring agents may be provided in the pharmaceutical composition. Examples of preservatives include sodium benzoate, sorbic acid and esters of p hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be administered by a number of routes.

The routes for administration (delivery) include, but are not limited to, one or more of: oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Where the composition is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

If a component of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134ATM) or 1,1,1,2,3,3,3-heptafluoropropane (HFA 227EATM), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the component of the present invention can be administered in the form of a suppository or pessary, or it may be applied topically in the form of a gel, hydrogel, lotion, solution, cream, ointment or dusting powder. The component of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the component of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, diet, mode and time of administration, rate of excretion, drug combination, the severity of the particular condition, and the individual undergoing therapy.

The component of the present invention may be formulated into a pharmaceutical composition, such as by mixing with one or more of a suitable carrier, diluent or excipient, by using techniques that are known in the art.

The composition may also be administered via the peripheral blood, for example by using skin patches.

Proteins and peptides may be delivered to a patient using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The protein and peptide can be administered by a surgically implanted device that releases the drug directly to the required site. For example, Vitrasert releases ganciclovir directly into the eye to treat CMV retinitis. The direct application of this toxic agent to the site of disease achieves effective therapy without the drug' significant systemic side-effects.

Electroporation therapy (EPT) systems can also be employed for the administration of proteins and peptides. A device which delivers a pulsed electric field to cells increases the permeability of the cell membranes to the drug, resulting in a significant enhancement of intracellular drug delivery.

Proteins and peptides can be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of protein and peptide delivery is the ReGel injectable system that is thermo-sensitive. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Protein and peptide pharmaceuticals can also be delivered orally. The process employs a natural process for oral uptake of vitamin B12 in the body to co-deliver proteins and peptides. By riding the vitamin B12 uptake system, the protein or peptide can move through the intestinal wall. Complexes are synthesised between vitamin B12 analogues and the drug that retain both significant affinity for intrinsic factor (IF) in the vitamin B12 portion of the complex and significant bioactivity of the drug portion of the complex.

Proteins and polypeptides can be introduced to cells by “Trojan peptides”. These are a class of polypeptides called penetratins which have translocating properties and are capable of carrying hydrophilic compounds across the plasma membrane. This system allows direct targeting of oligopeptides to the cytoplasm and nucleus, and may be non-cell type specific and highly efficient. See Derossi et al. (1998), Trends Cell Biol 8, 84-87.

If the agent is a protein, the protein may be prepared in situ in the subject being treated. In this respect, a polynucleotide encoding the agent may be delivered by use of non-viral techniques and/or viral techniques (both of which are described below) such that the protein is expressed from the polynucleotide. Similarly, if the agent itself is a polynucleotide, it may be administered using any suitable technique. The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

Polynucleotides may be administered systemically. Alternatively the inherent binding specificity of polynucleotides characteristic of base pairing is enhanced by limiting the availability of the polynucleotide to its intended locus in vivo, permitting lower dosages to be used and minimising systemic effects. Thus, polynucleotides may be applied locally to achieve the desired effect. The concentration of the polynucleotides at the desired locus is much higher than if the polynucleotides were administered systemically, and the therapeutic effect can be achieved using a significantly lower total amount. The local high concentration of polynucleotides enhances penetration of the targeted cells and effectively blocks translation of the target nucleic acid sequences.

The polynucleotides can be delivered to the locus by any means appropriate for localised administration of a drug. For example, a solution of the polynucleotides can be injected directly to the site or can be delivered by infusion using an infusion pump. The polynucleotides also can be incorporated into an implantable device which when placed adjacent to the desired site, to permit the polynucleotides to be released into the surrounding locus.

The polynucleotides may be administered via a hydrogel material. The hydrogel is non-inflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10% to about 80% by weight ethylene oxide and from about 20% to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename PluronicR.

In this embodiment, the hydrogel is cooled to a liquid state and the oligonucleotides are admixed into the liquid to a concentration of about 1 mg polynucleotides per gram of hydrogel. The resulting mixture then is applied onto the surface to be treated, for example by spraying or painting during surgery or using a catheter or endoscopic procedures. As the polymer warms, it solidifies to form a gel, and the polynucleotides diffuse out of the gel into the surrounding cells over a period of time defined by the exact composition of the gel.

The polynucleotides can be administered by means of other implants that are commercially available or described in the scientific literature, including liposomes, microcapsules and implantable devices. For example, implants made of biodegradable materials such as polyanhydrides, polyorthoesters, polylactic acid and polyglycolic acid and copolymers thereof, collagen, and protein polymers, or non-biodegradable materials such as ethylenevinyl acetate (EVAc), polyvinyl acetate, ethylene vinyl alcohol, and derivatives thereof can be used to locally deliver the polynucleotides. The polynucleotides can be incorporated into the material as it is polymerised or solidified, using melt or solvent evaporation techniques, or mechanically mixed with the material. In one embodiment, the polynucleotides are mixed into or applied onto coatings for implantable devices such as dextran coated silica beads, stents, or catheters.

The dose of polynucleotides is dependent on the size of the polynucleotides and the purpose for which is it administered. In general, the range is calculated based on the surface area of tissue to be treated. The effective dose of polynucleotide is somewhat dependent on the length and chemical composition of the polynucleotides but is generally in the range of about 30 to 3000 μg per square centimetre of tissue surface area.

The polynucleotides may be administered by any effective method, for example, parenterally (e.g. intravenously, subcutaneously, intramuscularly) or by oral, nasal or other means which permit the oligonucleotides to access and circulate in the patient' bloodstream. Polynucleotides administered systemically preferably are given in addition to locally administered polynucleotides, but also have utility in the absence of local administration. A dosage in the range of from about 0.1 to about 10 grams per administration to an adult human generally will be effective for this purpose.

Preferably, the genetic construct is adapted for delivery to a human cell.

Means and methods of introducing a genetic construct into a cell in an animal body are known in the art. For example, the constructs of the invention may be introduced into cells by any convenient method, for example methods involving retroviruses, so that the construct is inserted into the genome of the cell. For example, in Kuriyama et al. (1991) Cell Struc. and Func. 16, 503-510 purified retroviruses are administered. Retroviral DNA constructs comprising a polynucleotide as described above may be made using methods well known in the art. To produce active retrovirus from such a construct it is usual to use an ecotropic psi2 packaging cell line grown in Dulbecco' modified Eagle' medium (DMEM) containing 10% foetal calf serum (FCS). Transfection of the cell line is conveniently by calcium phosphate co-precipitation, and stable transformants are selected by addition of G418 to a final concentration of 1 mg/ml (assuming the retroviral construct contains a neoR gene). Independent colonies are isolated and expanded and the culture supernatant removed, filtered through a 0.45 μm pore-size filter and stored at −70° C.

Alternatively, as described in Culver et al. (1992) Science 256, 1550-1552, cells which produce retroviruses are injected. The retrovirus-producing cells so introduced are engineered to actively produce retroviral vector particles so that continuous productions of the vector occurred within the tumour mass in situ. Thus, proliferating epidermal cells can be successfully transduced in vivo if mixed with retroviral vector-producing cells.

Targeted retroviruses are also available for use in the invention; for example, sequences conferring specific binding affinities may be engineered into pre-existing viral env genes (see Miller & Vile (1995) Faseb J. 9, 190-199 for a review of this and other targeted vectors for gene therapy).

Other methods involve simple delivery of the construct into the cell for expression therein either for a limited time or, following integration into the genome, for a longer time. An example of the latter approach includes liposomes (Nassander et al. (1992) Cancer Res. 52, 646-653).

For the preparation of immuno-liposomes MPB-PE (N-[4-(p-maleimidophenyl)butyryl]-phosphatidylethanolamine) is synthesised according to the method of Martin & Papahadjopoulos (1982) J. Biol. Chem. 257, 286-288. MPB-PE is incorporated into the liposomal bilayers to allow a covalent coupling of the antibody, or fragment thereof, to the liposomal surface. The liposome is conveniently loaded with the DNA or other genetic construct of the invention for delivery to the target cells, for example, by forming the liposomes in a solution of the DNA or other genetic construct, followed by sequential extrusion through polycarbonate membrane filters with 0.6 μm and 0.2 μm pore size under nitrogen pressures up to 0.8 MPa. After extrusion, entrapped DNA construct is separated from free DNA construct by ultracentrifugation at 80 000×g for 45 min. Freshly prepared MPB-PE-liposomes in deoxygenated buffer are mixed with freshly prepared antibody (or fragment thereof) and the coupling reactions are carried out in a nitrogen atmosphere at 4° C. under constant end over end rotation overnight. The immunoliposomes are separated from unconjugated antibodies by ultracentrifugation at 80,000×g for 45 minutes. Immuno-liposomes may be injected intraperitoneally or directly into the site where they are required, e.g. a tumour.

Other methods of delivery include adenoviruses carrying external DNA via an antibody-polylysine bridge (see Curiel Prog. Med. Virol. 40, 1-18) and transferrin-polycation conjugates as carriers (Wagner et al. (1990) Proc. Natl. Acad. Sci. USA 87, 3410-3414). In the first of these methods a polycation-antibody complex is formed with the DNA construct or other genetic construct of the invention, wherein the antibody is specific for either wild-type adenovirus or a variant adenovirus in which a new epitope has been introduced which binds the antibody. The polycation moiety binds the DNA via electrostatic interactions with the phosphate backbone.

The adenovirus, because it contains unaltered fibre and penton proteins, is internalised into the cell and carries into the cell with it the DNA construct of the invention. It is preferred if the polycation is polylysine.

The DNA may also be delivered by adenovirus wherein it is present within the adenovirus particle, for example, as described below.

In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulfide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the tumour cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al. (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle.

It will be appreciated that “naked DNA” and DNA complexed with cationic and neutral lipids may also be useful in introducing the DNA of the invention into cells of the individual to be treated. Non-viral approaches to gene therapy are described in Ledley (1995) Human Gene Therapy 6, 1129-1144.

Alternative targeted delivery systems are also known such as the modified adenovirus system described in WO 94/10323 wherein, typically, the DNA is carried within the adenovirus, or adenovirus-like, particle. Michael et al. (1995) Gene Therapy 2, 660-668 describes modification of adenovirus to add a cell-selective moiety into a fibre protein. Mutant adenoviruses which replicate selectively in p53-deficient human tumour cells, such as those described in Bischoff et al. (1996) Science 274, 373-376 are also useful for delivering the genetic construct of the invention to a cell. Thus, it will be appreciated that a further aspect of the invention provides a virus or virus-like particle comprising a genetic construct of the invention. Other suitable viruses or virus-like particles include HSV, AAV, vaccinia and parvovirus.

Further aspects of the present invention relate to screening methods for agents such as drugs for inhibiting or activating type I PIP kinase or type II PIP kinase activity, or lead compounds for the development of drugs that inhibit or activate type I PIP kinase or type II PIP kinase activity.

The type I PIP kinase or type II PIP kinases used in the screening methods and assays may be type I PIP kinase or type II PIP kinases as defined above in the first aspect of the invention, including all isoforms of the enzymes. However, it is appreciated that the type I PIP kinase or type II PIP kinases used in the screening methods may be a fragment, variant, derivative or fusion of type I PIP kinase or type II PIP kinase, providing that it retains an activity of type I PIP kinase or type II PIP kinase. Activity of these kinases is as described herein. In one example, a fragment, variant, derivative or fusion retains a phosphorylation activity of type I PIP kinase or type II PIP kinase. The phosphorylation activity of type I PIP kinase typically includes, for example, phosphorylation of: PtdIns4P, PtdIns(3,4)P₂, PtdIns3P and PtdIns, wherein PtdIns4P is the preferred substrate. The phosphorylation activity of type II PIP kinase typically includes, for example, phosphorylation of: PtdIns3P, PtdIns(3,5)P₂PtdIns and PtdIns5P, wherein PtdIns5P is the preferred substrate.

An agent tested in these screening methods may be an organic compound or another chemical. The agent includes, but is not limited to, a compound which may be obtainable from or produced by any suitable source, whether natural or not. The agent can be a peptide or polypeptide, or a chemical derivative thereof, or a combination thereof. The agent may even be a nucleotide sequence—which may be a sense sequence or an anti-sense sequence.

The agent may be designed or obtained from a library of compounds which may comprise peptides, as well as other compounds, such as small organic molecules, such as lead compounds.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptides synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof.

It is contemplated that in future further compounds which are able to inhibit type I PIP kinase or type II PIP kinase activity will be discovered, such as low molecular weight organic molecules. These are considered to be within the scope of the present invention.

The agent may be in the form of a pharmaceutically acceptable salt—such as an acid addition salt or a base salt—or a solvate thereof, including a hydrate thereof. For a review on suitable salts see Berge et al., J. Pharm. Sci., 1977, 66, 1-19.

The agents may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided they retain the appropriate functional activity (though not necessarily to the same degree).

The agent for use in the present invention may exist in polymorphic form.

It will be appreciated by those skilled in the art that the agent for use in the present invention may be derived from a prodrug. Examples of prodrugs include entities that have certain protected group and which may not possess pharmacological activity as such, but may, in certain instances, be administered (such as orally or parenterally) and thereafter metabolised in the body to form the agent of the present invention which are pharmacologically active.

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the agents. Such prodrugs are also included within the scope of the invention.

The present invention also includes the use of zwitterionic forms of the agent for use in the present invention. The terms used in the claims encompass one or more of the forms just mentioned.

The present invention also includes the use of solvate forms of the agent for use in the present invention. The terms used in the claims encompass these forms.

The term “derivative” or “derivatised” as used herein includes chemical modification of an agent. Illustrative of such chemical modifications would be replacement of hydrogen by a halo group, an alkyl group, an acyl group or an amino group.

In one embodiment of the present invention, the agent may be a chemically modified agent. The chemical modification of an agent of the present invention may either enhance or reduce hydrogen bonding interaction, charge interaction, hydrophobic interaction, Van Der Waals interaction or dipole interaction between the agent and the target.

It will be appreciated that in the screening methods described herein, the agent identified may be a drug-like compound or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons and which may be water-soluble. A drug-like compound may additionally exhibit features of selective interaction with a particular protein or proteins and be bioavailable and/or able to penetrate target cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

Alternatively, the methods may be used as “library screening” methods, a term well known to those skilled in the art. Thus, for example, the methods of the invention may be used to detect (and optionally identify) a polynucleotide capable of expressing a polypeptide activator of type I PIP kinase or type II PIP kinase. Aliquots of an expression library in a suitable vector may be tested for the ability to give the required result.

It will be appreciated that several cycles of identifying pools of polynucleotides comprising a polynucleotide having the required property and then rescreening those polynucleotides may be required in order to identify a single species of polynucleotide with the required property. Methods of preparing a suitable expression library for screening are well known to those skilled in the art.

A further aspect of the invention provides a method of identifying a drug-like compound or lead compound for the development of a drug-like compound that modulates the activity of type I PIP kinase or type II PIP kinase, the method comprising contacting a compound with type I PIP kinase or type II PIP kinase or a suitable variant, fragment, derivative or fusion thereof or a fusion of a variant, fragment or derivative thereof and determining whether, an activity of type I PIP kinase or type II PIP kinase (or variant, fragment, derivative or fusion thereof or a fusion of a variant, fragment or derivative thereof) is changed compared to the its activity in the absence of the compound.

It will be understood that it will be desirable to identify compounds that may inhibit or activate the activity of type I PIP kinase or type II PIP kinase in vivo.

It will be appreciated that screening assays which are capable of high throughput operation will be particularly preferred. Examples may include cell based assays and protein-protein binding assays. An SPA-based (Scintillation Proximity Assay; Amersham International) system may be used. For example, an assay for identifying a compound capable of modulating the activity of a protein kinase may be performed as follows. Beads comprising scintillant and a polypeptide that may be phosphorylated may be prepared. The beads may be mixed with a sample comprising the protein kinase and 32P-ATP or 33P-ATP and with the test compound. Conveniently this is done in a 96-well or 384-well format. The plate is then counted using a suitable scintillation counter, using known parameters for 32P or 33P SPA assays. Only 32P or 33P that is in proximity to the scintillant, i.e. only the isotope bound to the polypeptide, is detected. Variants of such an assay, for example in which type I PIP kinase (or alternatively type II PIP kinase) is immobilised on the scintillant beads via binding to an antibody, may also be used.

Other methods of detecting polypeptide/polypeptide interactions include ultrafiltration with ion spray mass spectroscopy/HPLC methods or other physical and analytical methods. Fluorescence Energy Resonance Transfer (FRET) methods, for example, well known to those skilled in the art, may be used, in which binding of two fluorescent labelled entities may be measured by measuring the interaction of the fluorescent labels when in close proximity to each other.

Alternative methods of detecting binding of a polypeptide to macromolecules, for example DNA, RNA, proteins and phospholipids, include a surface plasmon resonance assay, for example as described in Plant et al. (1995) Analyt Biochem 226(2), 342-348. Methods may make use of a polypeptide that is labelled, for example with a radioactive or fluorescent label.

A further method of identifying a compound that is capable of binding to type I PIP kinase or type II PIP kinase is one where the polypeptide is exposed to the compound and any binding of the compound to type I PIP kinase or type II PIP kinase is detected and/or measured. The binding constant for the binding of the compound to the polypeptide may be determined. Suitable methods for detecting and/or measuring (quantifying) the binding of a compound to a polypeptide are well known to those skilled in the art and may be performed, for example, using a method capable of high throughput operation, for example a chip-based method. Technology called VLSIPS™, has enabled the production of extremely small chips that contain hundreds of thousands or more of different molecular probes. These biological chips or arrays have probes arranged in arrays, each probe assigned a specific location. Biological chips have been produced in which each location has a scale of, for example, ten microns. The chips can be used to determine whether target molecules interact with any of the probes on the chip. After exposing the array to target molecules under selected test conditions, scanning devices can examine each location in the array and determine whether a target molecule has interacted with the probe at that location.

Biological chips or arrays are useful in a variety of screening techniques for obtaining information about either the probes or the target molecules. For example, a library of peptides can be used as probes to screen for drugs. The peptides can be exposed to a receptor, and those probes that bind to the receptor can be identified. See U.S. Pat. No. 5,874,219 issued 23 Feb. 1999 to Rava et al.

The agent identified by the screening methods described above may not itself be optimal for use in a pharmaceutical or medical context. The identified agent may be a lead-compound for the identification of further agents that would be more suitable for such uses. The invention therefore includes modifying an agent identified as a result of the screening methods described above, or taking a further compound having or expected to have similar properties to an agent identified as a result of the screening methods, and screening the modified agent or further compound as described above.

Typically, the test agents which have the desired effects in the above assays are selected for further investigation. Preferably, they are screened further, for example in a cell and/or animal model of a disorder and test agents are selected from these assays for further study if they are seen to have a desirable effect in the further screen.

A modulator of PIPI kinase or PIPII kinase expression may act at any level of expression. Accordingly a modulator may affect transcription, translation or post-translational processing. A modulator may affect the activity of the kinase.

A preferred aspect of the invention provides for a method for identifying an agent that inhibits or activates type I PIP kinase expression, the method comprising:

I. Providing a polynucleotide construct comprising a promoter of a gene encoding type I PIP kinase, or a functional equivalent thereof, operably linked to a coding sequence;
II. Providing a test agent;
Contacting a test substance with the polynucleotide construct under conditions that, in the absence of the test agent, would permit expression of the polypeptide encoded by the coding sequence;
IV. Determining the level of expression of the polypeptide encoded by the coding sequence, thereby determining whether the test agent inhibits or activates the expression of the polypeptide.

Wherein a change in the expression of the detectable product in the presence of the test agent indicates that the test agent may inhibit or activate type I PIP kinase gene expression.

A further aspect of the invention provides for a method for identifying an agent that inhibits or activates type II PIP kinase expression, the method comprising:

I. Providing a polynucleotide construct comprising a promoter of a gene encoding type II PIP kinase, or a functional equivalent thereof, operably linked to a coding sequence;
II. Providing a test agent;
III. Contacting a test substance with the polynucleotide construct under conditions that, in the absence of the test agent, would permit expression of the polypeptide encoded by the coding sequence;
IV. Determining the level of expression of the polypeptide encoded by the coding sequence, thereby determining whether the test agent inhibits or activates the expression of the polypeptide.

Wherein a change in the expression of the detectable product in the presence of the test agent indicates that the test agent may inhibit or activate type II PIP kinase gene expression.

In one aspect the methods are for identifying agents that inhibit expression.

The detectable product could be an RNA or polypeptide product. Suitable detectable products are well known in the art.

Typically, a decrease in expression of the detectable product indicates that the agent may be an inhibitor of type I PIP kinase or type II PIP kinase gene expression. Suitable techniques for measuring levels of RNA or polypeptides products are well known in the art.

In one embodiment, the detectable product may be type I PIP kinase or type II PIP kinase RNA or polypeptide.

A further aspect of the invention provides for a method for identifying an agent that inhibits or activates type I PIP kinase activity, the method comprising:

I. Contacting a test agent with an assay composition under conditions that, in the absence of the test substance, would allow type I PIP kinase activity;
II. Determining the level of activity of the type I PIP kinase thereby determining whether the test agent inhibits or activates the activity of type I PIP kinase.

A preferred aspect of the invention is a method as described above whereby the assay composition comprises a reaction mixture of:
A fluorescently labelled product or product analogue of type I PIP kinase bound to phospholipase C or a fragment thereof,
type I PIP kinase, or a fragment thereof;
and an unlabelled substrate of type I PIP kinase or unlabelled substrate analogue thereof.
   and whereby the activity of type I PIP kinase is indicated by a decrease in the fluorescent signal of the labelled product analogue.

A further aspect of the invention provides for a method for identifying an agent that inhibits or activates type II PIP kinase activity, the method comprising:

I. Contacting a test agent with an assay composition under conditions that, in the absence of the test substance, would allow type II PIP kinase activity;
II. Determining the level of activity of the type II PIP kinase thereby determining whether the test agent inhibits or activates the activity of type II PIP kinase.

A preferred aspect of the invention is a method as described above whereby the assay composition comprises a reaction mixture of:
A fluorescently labelled product or product analogue of type II PIP kinase bound to phospholipase C or a fragment thereof,
type II PIP kinase, or a fragment thereof;
and an unlabelled substrate of type II PIP kinase or unlabelled substrate analogue thereof.
   and whereby the activity of type II PIP kinase is indicated by a decrease in the fluorescent signal of the labelled product analogue.

The principle of the fluorescence assay is set out in FIG. 22 (the substrate shown is for type I PIP kinase (PIP5K) but the assay principle is applicable to type I PIP kinase (PIP5K) or type II PIP kinase (PIP4K) in any isoform. The pleckstrin homology (PH) domain of a PtdIns processing enzyme, phospholipase C delta binds strongly to the product of the PIPKIN reaction (PtdIns(4,5)P₂). The PH domain also has a higher affinity for PtdIns(4,5)P₂ (product) over PtdIns5P or PtdIns4P (substrates).

The assay uses a complex formed of a fluorescently tagged product analogue (e.g. PtdIns(4,5)P₂—F) bound to the PH domain of phospholipase C. This complex has a high fluorescence polarisation (FP) value as it is a large molecule.

When the complex is added to a completed type I PIP kinase (PIP5K) or type II PIP kinase (PIP4K) reaction, unlabelled PtdIns(4,5)P₂ product competes with the labelled molecules for binding to the PH domain. Displaced PtdIns(4,5)P₂—F exhibits a low FP value. This decrease in the FP measured for the PtdIns(4,5)P₂—F is proportional to the amount of PtdIns(4,5) P₂ produced and therefore proportional to the PIP5K or PIP4K activity.

Test agents or substances for screening in the assay are as described herein.

Type I PIP kinase or type II PIP kinases for use in screening methods such as the fluorescence assay have been described herein. As described, an assay may comprise use of a fragment, variant, derivative or fusion of type I PIP kinase or type II PIP kinase, providing that it retains an activity of type I PIP kinase or type II PIP kinase. Activity of these kinases is as described herein. In one example, a fragment, variant, derivative or fusion retains a phosphorylation activity of type I PIP kinase or type II PIP kinase, in particular the specific lipid kinase activity of the enzyme. The phosphorylation activity of type I PIP kinase typically includes, for example, phosphorylation of: PtdIns4P, PtdIns(3,4)P₂, PtdIns3P and PtdIns, wherein PtdIns4P is the preferred substrate. The phosphorylation activity of type II PIP kinase typically includes, for example, phosphorylation of: PtdIns3P, PtdIns(3,5)P₂PtdIns and PtdIns5P, wherein PtdIns5P is the preferred substrate.

An assay may comprise use of a polynucleotide encoding type I or type II PIP kinase as described herein. Expression of the polynucleotide provides the type I or type II PIP kinase activity for the assay.

Typically the fluorescently labelled product is F—PtdIns(4,5)P₂. A suitable analogue may also be used as described herein. Any suitable fluorophore or fluororescent label may be used for labelling. Means for labelling molecules fluorescently are known in the art.

The labelled product or analogue is generally bound to phospholipase C, a PtdIns processing enzyme, or a fragment thereof comprising the pleckstrin homology (PH domain). It has been shown that the PH domain of phospholipase C delta binds strongly to the product of the PIPKIN reaction (PtdIns(4,5)P). J Biol. Chem. 2004 Jun. 4; 279(23):24362-71. Epub 2004 March 22). The sequence of phospholipase C delta is found in Genbank Accession No. BC050382 and sequence is listed in FIG. 19.

Where the enzyme is type I PIP kinase (PIP5K), the substrate is generally selected from the following group: PtdIns4P, PtdIns(3,4)P₂, PtdIns3P and PtdIns or an analogue thereof wherein PtdIns4P is the preferred substrate. Preferred analogues are analogues based on inositol(1,4)bis phosphate which lack the fatty acid chain.

Where the enzyme is type II PIP kinase (PIP4K), the substrate is generally selected from the following group: PtdIns3P, PtdIns(3,5)P₂PtdIns and PtdIns5P or an analogue thereof wherein PtdIns5P is the preferred substrate. Preferred analogues are analogues based on inositol(1,4)bis phosphate which lack the fatty acid chain.

The assay composition will in general comprise other components such that, in the absence of the test substance, type II PIP kinase (PIP4K) kinase activity or type I PIP kinase (PIP5K) activity is permitted.

For example, the composition generally comprises a source of ATP, e.g. ATP disodium salt. A composition may also include appropriate buffer.

Assay components may be mixed in any order. Thus the method comprises admixing enzyme preparation, unlabelled substrate and labelled product, in the presence and absence of a test substance, incubating and determining the fluorescent reading. Preferably the reactions in the absence of the test substance are carried out simultaneously with the mixtures in the presence of the test substance. Typically the method comprises also carrying out suitable controls, e.g. in the absence of the enzyme.

The present assay may be used to screen multiple test substances in parallel. Test reactions can be carried out simultaneously (but separately) and results analysed in parallel. Typically such an assay uses apparatus having multiple separate reaction vessels. For example, reactions may be carried out in the wells of a multiwell plate, as described in the present Examples.

Thus the present assay has the advantage that it is suitable for high throughput screening of test substances.

Methods for determining fluorescence are known in the art. For example, where reactions are carried out in multiwell plates, a suitable plate reader, such as an Analyst HTS plate reader may be used.

Type II PIP kinase (PIP4K) or type I PIP kinase (PIP5K) kinase activity is indicated by a drop in the fluorescence polarisation value in the assay. The effect of a test agent on the kinase activity can be measured by comparing the rate of the signal decrease in the presence of said agent with the rate of the signal decrease in the absence of said agent.

Typically, a decrease in the activity of type I PIP kinase or type II PIP kinase indicates that the agent may be an inhibitor of the type I PIP kinase or type II PIP kinase, whereas an increase in the activity of type I PIP kinase or type II PIP kinase indicates that the agent may be an activator of the type I PIP kinase or type II PIP kinase.

An agent identified according to the present assays may inhibit or enhance PIP kinase activity as defined herein. For example, an agent may produce a low, medium or high level increase of decrease in activity.

Other methods of assessing the type I PIP kinase and type II PIP kinase activity are well known in the art for example described by Itoh et al, 1998. This and similar methods could be easily adapted by the skilled person in the art. Preferably these methods are adapted to allow for high through put screening of potential test agents.

A preferred aspect of the invention is a method as described above whereby the agent identified is modified to improve its activity and pharmaceutical properties and retested.

The invention also includes holding pre-clinical and clinical trials of an agent identified as a result of any of the above screening methods.

A preferred aspect of the invention is a method as described above which further comprises:

I. Contacting the agent identified with a mammalian cell;
II. Determining the efficacy of the agent for a disorder selected from the group of a hyperproliferative disorder, a neurodegenerative order or cardiac reperfusion injury.

An agent may, for example, be assayed for an ability to affect (increase or decrease) cellular PtdIns(4,5)P₂ levels.

An agent may, for example, be assayed for an effect on cellular apoptosis such cellular response to apoptotic agents e.g. H₂O₂ or UV irradiation, or for an effect on neurite outgrowth. For example, a candidate inhibitory agent may be assayed for an activity that increases apoptosis or sensitivity to apoptotic agents in the cell, or that stimulates neurite outgrowth. A candidate activatory agent may be assayed for an activity that reduces apoptosis or sensitivity to apoptotic agents in the cell, or that reduces neurite outgrowth.

A preferred aspect of the invention is a method as described above which further comprises:

I. Testing the agent identified in an animal model;
II. Determining the efficacy of the agent for a disorder selected from the group of a hyperproliferative disorder, a neurodegenerative order or cardiac reperfusion injury.

Suitable animal models are known in the art.

A preferred aspect of the invention is a method as described above which further comprises:

I. Testing the agent identified in a clinical trial for safety;
II. Determining the efficacy of the agent for a disorder selected from the group of a hyperproliferative disorder, a neurodegenerative order or cardiac reperfusion injury.

The identification methods described herein may be used for identifying agents that may be used to modulate cellular apoptosis or neurite outgrowth, or to treat or prevent hyperproliferative disorders, neurodegenerative disorders, or cardiac reperfusion.

A further aspect of the invention is an agent identifiable by any of the screening methods described herein. Typically, the agent is a compound such as a polypeptide, polynucleotide, or a small molecule, preferably an organic molecule. Preferably, small molecules are of less than 5000 daltons, and may be water-soluble.

A preferred embodiment of the invention is a method for the preparation of a pharmaceutical composition, comprising identifying an agent that inhibits or activates type I PIP kinase by a method as described above and formulating the agent with a pharmaceutically acceptable carrier thereof.

A preferred embodiment of the invention is a method for the preparation of a pharmaceutical composition, comprising identifying an agent that inhibits or activates type II PIP kinase by a method as described above and formulating the agent with a pharmaceutically acceptable carrier thereof.

The invention further includes packaging and presenting an agent identified as a result of any of the above screening methods for use in medicine as a pharmaceutical composition comprising an agent as defined above and a pharmaceutically acceptable carrier, diluent or excipient (including combinations thereof).

A further embodiment of the present invention is a method for the treatment of an individual suffering from a hyperproliferative disorder, which method comprises administering to the individual a therapeutically effective amount of an inhibitor agent identified by the methods as defined above.

As described herein, the invention in one aspect relates to agents which are capable of modulating the levels of PtdIns(4,5)P₂ in a cell. An agent may increase or decrease levels of PtdIns(4,5)P₂. An agent which increases PtdIns(4,5)P₂ levels typically increases expression and/or activity or type I or type II PIP kinase. An agent which decreases PtdIns(4,5)P₂ levels typically decreases or inhibits expression and/or activity of type I or type II PIP kinase. Levels of increase or decrease are as described herein. Suitable agents are described herein and/or may be identified according to the methods described herein.

In one aspect the invention relates to any one or more of these agents for use in medicine, as described herein.

In particular, the invention relates to an agent which is capable of decreasing PtdIns(4,5)P₂ levels as described herein for use in inducing apoptosis or sensitising a cell to apoptotic agents. The invention relates to such an agent for use in treating or preventing a hyperproliferative disorder such as those described herein. The invention further relates to such an agent for use in stimulating neurite outgrowth, and for treating a neurodegenerative disorder such as those described herein. The invention further relates to the use of such an agent for the manufacture of a medicament for use in any of these methods.

The invention also relates to an agent which is capable of increasing PtdIns(4,5)P2 levels as described herein for use in preventing apoptosis, and for use in treating diseases such as cardiac reperfusion or neurodegenerative diseases. The invention further relates to use of such an agent for the manufacture of a medicament for use in any of these methods.

Methods for formulating agents in compositions and methods for administration are as described herein.

The methods described herein, for example for modulating apoptosis, sensitising cells to apoptotic agents or stimulating neurite outgrowth may be carried out in vivo or in vitro.

### DESCRIPTION OF THE FIGURES

FIG. 1. A) Murine erythroleukaemia (MEL) cells were irradiated with UV light (200J/m) or gamma radiation (10 Gy). After 30 minutes the cells were fractionated into a cytosol/membrane mixture and nuclei. A mass assay was used to determine the levels of PtdIns4P and PtdIns5P. The results are expressed as % of control-irradiated cells and are calculated from 3 independent experiments.

B) Differentiated MEL cells were treated with NAC (5 mM) or BSO (100 μM), each for 4 hours, or H₂O₂ (500 μM) for 15 minutes prior to the isolation of intact nuclei. A mass assay was used to determine the levels of PtdIns4P and PtdIns5P. The results are expressed as % of control-treated differentiated MEL cells and are calculated from 3 independent experiments.

C) A mass assay was used to determine the levels of PtdIns4P and PtdIns5P in the chromatin enriched fraction (CEF) from MEL cells irradiated with UV light (200 J/m) or gamma radiation (10 Gy). The results are expressed as % of control-irradiated cells and are calculated from 3 independent experiments. HT1080 cells were treated with etoposide (100 μM) or vehicle (DMSO) for 4 hours before the isolation of the CEF. The level of both PtdIns4P and PtdIns5P was determined using the mass assay. The results are expressed as % of vehicle-treated cells and are calculated from 3 independent experiments. Inset: MEL cells were irradiated with UV light (200 J/m). After 30 minutes the cells were fractionated into a cytosol/membrane mixture and intact nuclei. The latter was further fractionated into nuceloplasm and the CEF. Western blotting for actin, tubulin and histone 4 proteins was used for checking the purity of the fractions. Equal quantities of protein were loaded from each subcellular fraction. Correcting for cell equivalents, reveals that the amount of actin in the cytosol/membrane mixture is at least two orders of magnitude higher that found in the nucleoplasm and CEF, as expected. A representative western blot is shown.

D) MEL cells were preincubated with either vehicle (DMSO) or wortmannin (200 nM and 5 μM) were irradiated with 200 J/m of UV light. After 30 minutes the CEF was isolated and a mass assay was used to determine the levels of PtdIns4P and PtdIns5P (expressed as % of control non-irradiated cells. The results are calculated from 3 independent experiments).

E) MEL cells were preincubated with either vehicle (ethanol) or curcumin (100 μM) were irradiated with 200 J/m of UV irradiation. After 30 minutes the CEF was isolated and a mass assay was used to determine the levels of PtdIns4P and PtdIns5P (expressed as % of control, non curcumin-treated nor non UV-irradiated cells. The results are calculated from 3 independent experiments).

FIG. 2. A) MEL cells were transiently transfected to express EGFP (green fluorescent protein) and EGFP-type II b PIP kinase. Intact nuclei were isolated from the cells after 40 hours. The presence of EGFP and EGFP-type II PIP kinase was determined by western blotting using a specific antibody against EGFP. The arrows indicate the position of the expressed proteins. A representative western blot is shown.

B) MEL cells were transiently transfected with either pSuper or pSuper-IIβRNAi. After 40 hours intact nuclei were isolated from the cells. The level of endogenous type II b PIP kinase was determined by western blotting using a specific antibody. As a control for loading, histone 4 was detected in the same samples. A representative western blot is shown.

C) MEL cells were transiently transfected to express EGFP, EGFP-type II b PIP kinase and EGFP-IpgD/IpgE. In addition MEL cells were transfected with pSuper-IIβRNAi. After 40 hours the cells some of the cells were irradiated with 200 J/m of UV light. 30 minutes later the CEF was isolated. A mass assay was used to determine the level of PtdIns5P (expressed as % of control non-irradiated cells. The results are calculated from 3 independent experiments).

FIG. 3. A) MEL cells were either untransfected or transfected to express myc-type II b PIP kinase. After 40 hours the cells some of the cells were irradiated with 200 J/m of UV light before reculturing for varying times indicated in the figure. The cells were collected and endogenous type II PIP kinase was immunoprecipitated from whole cell lysates using an anti-pan type II b PIP kinase antibody or an anti-myc antibody. Type II PIP kinase activity in the immunoprecipitates was determined using PtdIns5P as the substrate. The results are calculated from 3 independent experiments.

B) MEL cells were irradiated with 200 J/m of UV light followed by reculturing for varying times indicated in the figure. After cell lysis, cytosolic proteins were separated by SDS-PAGE before transfer to an Immobilon-P membrane. The phosphorylation state of p54, p46 and p38 was determined using phosphospecific antibodies. The panel also indicates the level of total p54, p46 and p38. Anisomycin (A, 10 μM, 30 minutes) was used as a positive control to induce p38 phosphorylation. Representative western blots are shown.

C) HEK293 cells were subjected to single plasmid transfections or cotransfections to express the proteins indicated in the panel. Myc type II b PIP kinase was immunoprecipitated from whole cell lysates using an anti-myc antibody. Type II PIP kinase activity in the immunoprecipitates was determined using PtdIns5P as the substrate. The expression of 3xflag-MKK6+ and 3xflag-MKK6− in total lysates and myc type II b PIP kinase in the immunopreciptates is indicated in the panel. The spot corresponding to where radiolabelled phosphatidylinositol 4,5-bisphosphate (PIP2) migrated on the thin layer chromatography autoradiography image is indicated. Representative western blots are shown. The results are representative of two experiments.

FIG. 4. A) Exponentially growing MEL cells were irradiated with either 200 J/m of UV light or 10 Gy of gamma radiation. After 30 minutes the CEF was isolated and the subjected to SDS-PAGE followed by transfer of the separated proteins to a nitrocellulose membrane. Western blotting for endogenous ING2 and histone 4 proteins was performed. Representative western blots are shown.

B) Exponentially growing MEL cells were incubated in the absence or presence of N-acetyl cysteine (NAC) (5 mM) for 4 hours prior to a control irradiation or exposed to 200 J/m of UV light before the isolation of the CEF. Differentiated MEL cells were incubated in the absence or presence of H₂O₂ (500 μM) for 15 minutes prior to the isolation of the CEF. Proteins in the CEF were subjected to SDS-PAGE followed by transfer to a nitrocellulose membrane. Western blotting for endogenous ING2 and histone 4 proteins was performed. Representative western blots are shown.

C) MEL cells were preincubated with either vehicle, wortmannin (200 nM and 5 μM) or curcumin (100 μM) were irradiated with 200 J/m of UV light. After 30 minutes the CEF was isolated and proteins were subjected to SDS-PAGE followed by transfer to a nitrocellulose membrane. Western blotting for endogenous ING2 and histone 4 proteins was performed. Representative western blots are shown.

D) The CEF from transiently transfected MEL cells with either pSuper or pSuper-IIβRNAi was prepared. Proteins in the CEF were subjected to SDS-PAGE followed by transfer to a nitrocellulose membrane. The CEF from control and UV-irradiated transiently transfected MEL cells (to express EGFP and EGFP-type II b PIP kinase) was prepared. Proteins in the CEF were subjected to SDS-PAGE followed by transfer to a nitrocellulose membrane. Western blotting for endogenous ING2 and histone 4 proteins was performed. Representative western blots are shown.

E) HT1080 cells were transfected with the indicated plasmids and cultured for a further 40 hours. The % of sub G0/G1 population cells was determined by flow cytometry. Expression levels of the indicated constructs were determined by western blotting. In the bottom panel HT1080 cells were transfected as indicated and after 24 hours the cells were treated with or without 25 μM etoposide. 40 hours later the % of cells in the sub G0/G1 population was determined. Data are represented as the means+/−the range for duplicate samples and similar results were found in two further independent experiments.

FIG. 5. The phosphoinositide binding characteristics of nine PHD motifs termed PHD-25, PHD-26, PHD-29, PHD-32, PHD33, PHD-36, PHD-37, PHD-38 and PHD-41 (bacterially expressed as GST-fusion proteins) were assessed using lipid dot blots. The phosphoinositide spot layout is indicated. The asterisk indicates natural PtdIns(4,5)P₂. Representative lipid dot blots from repeated cloning, expression, and purification of the GST fusion proteins are indicated in the panel.

B) A NCBI Blast alignment of amino acid sequences of 14 PHD motifs including that of ING2. Key lysine and arginine residues form a common sequence, as indicated by the black box.

FIG. 6. Selective binding of the pleckstrin homology (PH) domain to product. 10 nM of either fluorescent substrate (squares) or fluorescent product (circles) was mixed with increasing amounts of GST-tagged PH domain (Cloned and overexpressed from E. coli at CRT DL).

FIG. 7. A 10 nM-1 uM PtdIns(4,5)P—F PH domain complex was incubated with increasing concentrations of unlabelled PtdIns(4,5) P₂ (circles) and unlabelled PtdIns5P (squares). The unlabelled product disrupts the complex fully at 1 mM, substrate has no effect at 16 mM, (duplicates).

FIG. 8. Displacement of fluorescent PtdIns(4,5)P—F from the PH domain complex by the reaction products of PIPKIN. The PIPKIN assay was first run in the presence of the PK/LDH ATP regenerating system and then this mix was added to preformed PH domain-PtdIns(4,5)P—F complex and the mixture read for fluorescence polarisation (FP).

FIG. 9 shows that cells tightly regulate their PtdIn(4,5)P₂ levels. PtdIn(4,5)P₂ levels after stimulation with neurokinin A are visualised by GFP PH domain of PLC (Example 3). The figure shows that PtdIn(4,5)P₂ levels are rapidly recovered within minutes post-stimulation with growth factors. Panel A: In healthy cells with high levels of PtdIns(4,5) P₂ the probe remains at the plasma membrane.

Panel B: The picture shows that after growth factor stimulation the PtdIns(4,5) P₂ probe falls off the membrane.

Panel C: This effect is transient because the probe quickly goes back to the plasma membrane.

FIG. 10 shows that H₂O₂ sends cells into apoptosis by depleting PtdIn(4,5)P₂ level within cells (Example 4). HeLa cell were transiently transfected with an expression plasmid encoding a fusion protein of GFP and the pleckstrin homology domain of PLCdelta1. GFP PLC delta1 is a highly specific in vivo probe for PtdIns(4,5)P₂ in cells.

Panel A: In healthy cells with high levels of PtdIns(4,5) P₂ the probe remains at the plasma membrane.

Panel B: The picture shows that after cells are stimulated with an apoptotic concentration of H₂O₂ (600 microMolar). The probe rapidly translocates from the plasma membrane into the cytosol.

FIG. 11 shows that type I PIP kinase protects cells from ROS induced apoptosis. HeLa cell were transiently transfected with expression plasmids encoding for the listed proteins. Cell were then treated with 600 microMolar H₂O₂ for 24 hours. All cells were collected and examined for apoptosis (Example 5).

FIG. 12 shows that ROS target and negatively regulate type 1 PIP kinase (Example 5).

Panel A: HeLa cell were transiently transfected with expression plasmids encoding for a GFP fusion protein of PIP 5-K. In healthy cells GFP PIP 5-K is located at the plasma membrane. Cells were then treated with 600 microMolar H₂O₂ and the localisation of the GFP PIP 5-K was monitored on line using confocal laser scanning microscopy.

Panel B: Recombinant wild type PIP 5-K and a point mutation of PIP 5-K—(cysteine 175 altered to a serine residue) were both treated with H₂O₂ and enzyme activity was assayed in vitro by measuring the amount of product that is produced after incubation with substrate and radioactive ATP. The products were then analysed on TLC plate using phosphoimager.

FIG. 13

Panel A and B: Neuroblastoma cells (N2A) were transfected either with a control plasmid (GFP) or with a kinase inactive mutant of PIP 5-K alpha (which can no longer elevate PtdIns(4,5) P₂ levels) in the presence of serum. 24 hours after transfection morphology was recorded using conventional phase contrast microscopy (Example 6).

In panel C and D RNAi was used to knock out PIP 5-K alpha activity.

FIG. 14

The figure is a demonstration of the various models that can be used to screen the efficacy of PtdIns(4,5) P₂ inhibitors in cells.

Panel A: FRET technique for online PtdIns(4,5) P₂ measurement. What is shown is a typical FRET trace showing that PtdIns(4,5) P₂levels drop in a single cell after treatment with H₂O₂.

This can also be monitored using confocal microscopy (Panel B). Panel C shows the traditional method of following PtdIns(4,5) P₂ metabolism-orthophosphate labelling studies where we monitor PtdIns(4,5) P₂ decrease after H₂O₂ stimulation.

FIG. 15: DNA and amino acid sequence of rat type II PIP kinase.

FIG. 16: DNA and amino acid sequence of human type II PIP kinase

FIG. 17: DNA and amino acid sequence of mouse type I PIP kinase

FIG. 18: DNA and amino acid sequence of human type I PIP kinase

FIG. 19: DNA and amino acid sequence of humans phospholipase C, delta 1

FIG. 20: A PtdIns(4,5)P—F PH domain complex (10 nM/500 nM) was incubated with increasing concentrations of unlabelled PtdIns(4,5) P₂ (triangles) and unlabelled PtdIns4P (diamonds). The unlabelled product disrupts the complex fully at 750 nM, substrate has no effect at 5 μM.

FIG. 21: Selective binding of the PH domain to product. 10 nM of either fluorescent PI(4)P (squares) or fluorescent PI(5)P (triangles) or fluorescent product (diamonds) was mixed with increasing amounts of GST-tagged PH domain (Cloned and overexpressed from E. coli at CRT DL).

FIG. 22: A diagrammatic representation of the principle of the high throughput assay for PIP kinase activity. The same principle applies for the assay applied to PIP4K, but the substrate is PI5P.

FIG. 23: Displacement of fluorescent PtdIns(4,5)P—F from the PH domain complex by the reaction products of type I PIP kinase (PIP5K). The assay was first run in the presence of the PK/LDH ATP regenerating system and then this mix was added to preformed PH domain-PtdIns(4,5)P₂—F complex and the mixture read for FP. The assay was carried out for 3 PIP5K isoforms: PIP5K alpha, PIP5K gamma and PIP5K beta 8/6. A detailed assay protocol is given in Example 7.

### EXAMPLES

The following Examples illustrate the invention.

Unless indicated otherwise, the methods used are standard biochemistry and molecular biology techniques. Examples of suitable methodology textbooks include Sambrook et al; Molecular Cloning, A Laboratory Manual (1989) and Ausubel et al; Current Protocols in Molecular Biology (1995), John Wiley & Sons Inc.

### Materials and Methods

### Cell Culture, Transfection and Treatments.

MEL, HEK 293 and HT1080 cells were maintained in routine culture using 10% FCS in DMEM. When required, differentiated MEL cells were prepared by the addition of 1.5% DMSO to the culture medium for a period of 4 days. MEL cells were transfected using Effectene (Qiagen), HEK 293 cells with calcium phosphate and HT1080 with LT1 (Mirus corporation). The following plasmids were used: pEGFP, pCDNA3 (both from Clontech), pCDNA3-Flag-MKK6+ and pCDNA3-Flag-MKK6-(from Dr. M. Schmidt, The Netherlands Cancer Institute), pSuper (from Dr. R. Agami, The Netherlands Cancer Institute), pCDNA3-EGFP-IpgD/IpgE, pCDNA3-EGFP-PIP kinase typeIIβ, and RNAi for type II β PIP kinase (termed pSuper-IIβRNAi). The targeting sequence for the latter was cctccccagccgcttcaag. Irradiation of the cells with 254 nm UV-C light (0-200 J/m) or gamma rays (0-40 Gy, from a Cesium 137 source) was performed using 6-well or 10 cm plastic petri dishes with a depth of medium covering the cells not exceeding 3-4 mm. Thereafter, the cells were returned to normal culture conditions for 30 minutes before subcellular fractionation was started. To determine long terms effects of UV and gamma irradiation the cells were returned to normal culture conditions for up to 48 hours.

### Subcellular Fractionation and Phospholipid Analyses.

MEL cells were washed, twice in ice cold PBS before their fractionation using a detergent-free hypotonic buffer. This method yields a mixed cytosol and membrane fraction and intact nuclei after separation using a sucrose cushion. MEL cell nuclei were fractionated into nucleoplasm and the CEF using modifications of a previously published method. Briefly, intact MEL cell nuclei were resuspended in ice cold hypotonic buffer (3 mM EDTA, 0.2 mM EGTA and 1 mM DTT pH 7.85, with protease inhibitors) followed by forced passage (12 times) through a 25 gauge needle. After centrifugation (1700 g) the supernatant (the nucleoplasm) was saved. The pellet (CEF) was washed twice before resuspending by sonication in 10 mM TrisHCl, 10% sucrose, 10 mM MgCl₂ pH 7.4, with protease inhibitors. The protein content of all fractions was determined using the Bradford reagent (BioRad). PtdIns4P and PtdIns5P were measured using in vitro mass assays employing recombinant type I and type II PIP kinases, respectively. The product PtdIns(4,5)P₂ was separated by TLC. The amount of radioactivity found in PtdIns(4,5)P₂ was proportional to the amount of PtdInsP isomer in the neomycin affinity-purified lipid extract.

### Flow Cytometry and Western Blotting.

Ethanol fixed cells were incubated with prodium iodide (20 μg/ml) and RNAse (10 μg/ml) before cell cycle analysis by flow cytometry. Proteins were analysed by standard western blotting procedures employing the following antibodies: anti-tubulin and anti-flag (both from Sigma), anti-actin (Chemicon International), anti-histone 4 (Upstate Biotechnology), anti-phospho p54, anti-phospho p46, anti-phospho p38, total p54, total p46 and total p38 (all from Cell Signalling Technology) anti-ING2, anti EGFP (Clontech), anti-type II β PIP kinase, anti-GST and anti-myc (both generated in house).

### Lipid Dot Blots

cDNA encoding PHD motifs were generated using PCR and cloned directly in pGEX4T-1 (primers available on request). All pGEX clones were sequence verified. GST proteins were purified according to established protocols except that zinc chloride (10 μM) was included in all buffers. GST-fusion proteins were quantitated after SDS-PAGE using coomasie blue and were used for lipid dot blots at a final concentration of 100 ng/ml. Phospholipids (100 pmol/2 μl chloroform) were spotted onto nitrocellulose and blots were blocked using TBS-T (0.1%) with zinc chloride (10 μM). The blots were incubated overnight with the GST fusion protein and washed in the above buffer. After incubation with an anti-GST antibody followed by anti mouse-HRP, phosphoinositide-GST-fusion interactions were visualised using Super Signal (Pierce).

### Example 1

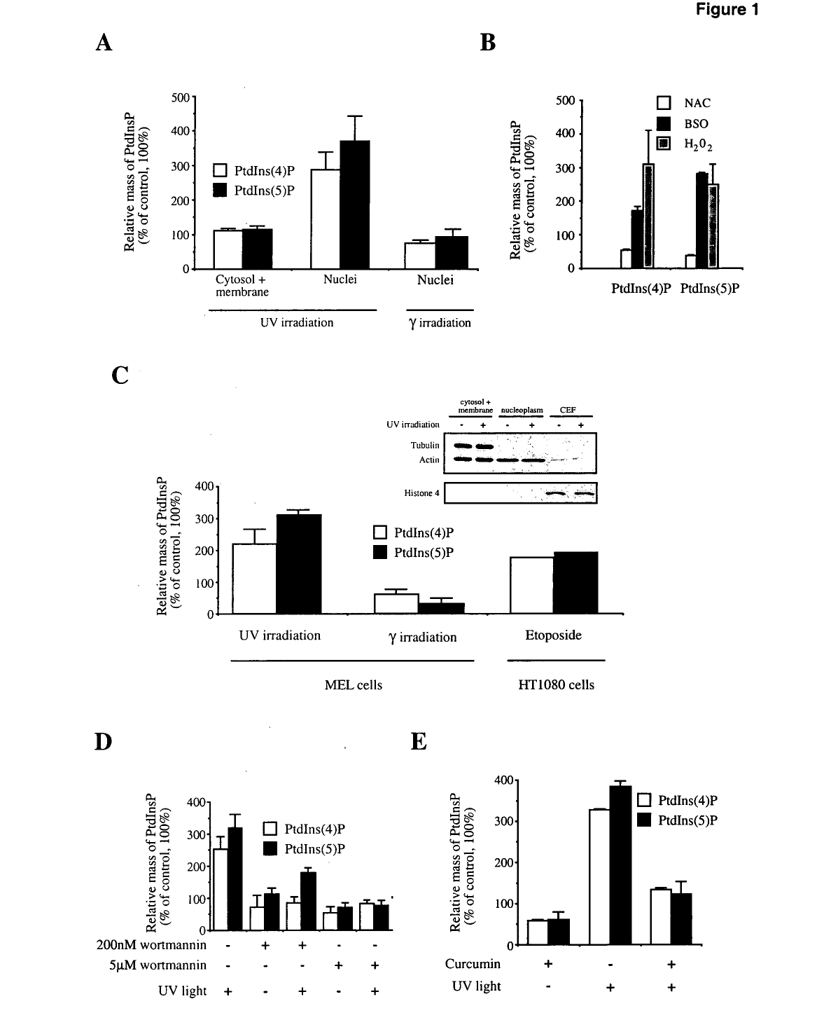

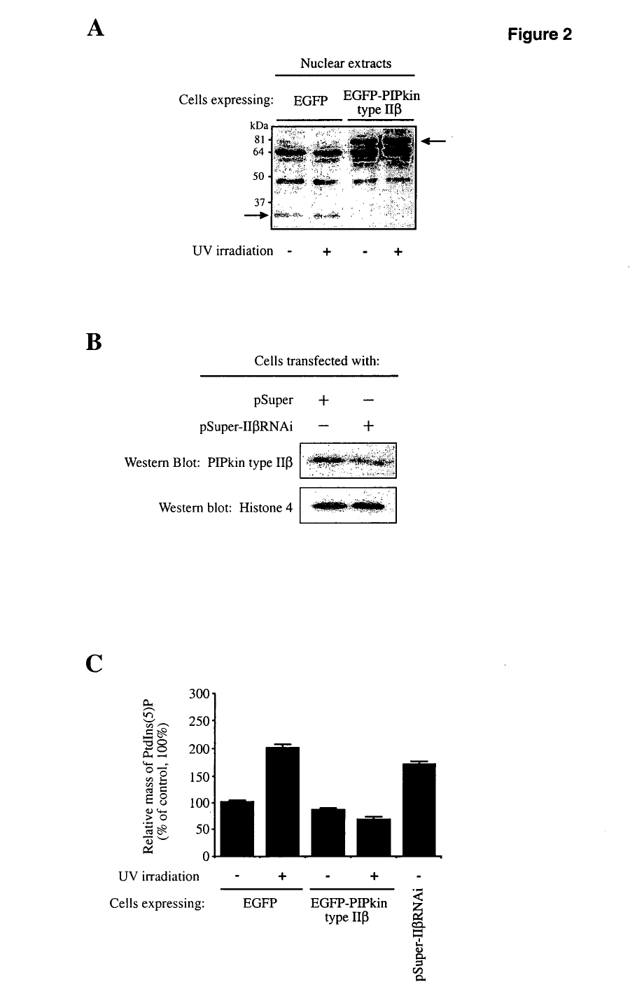

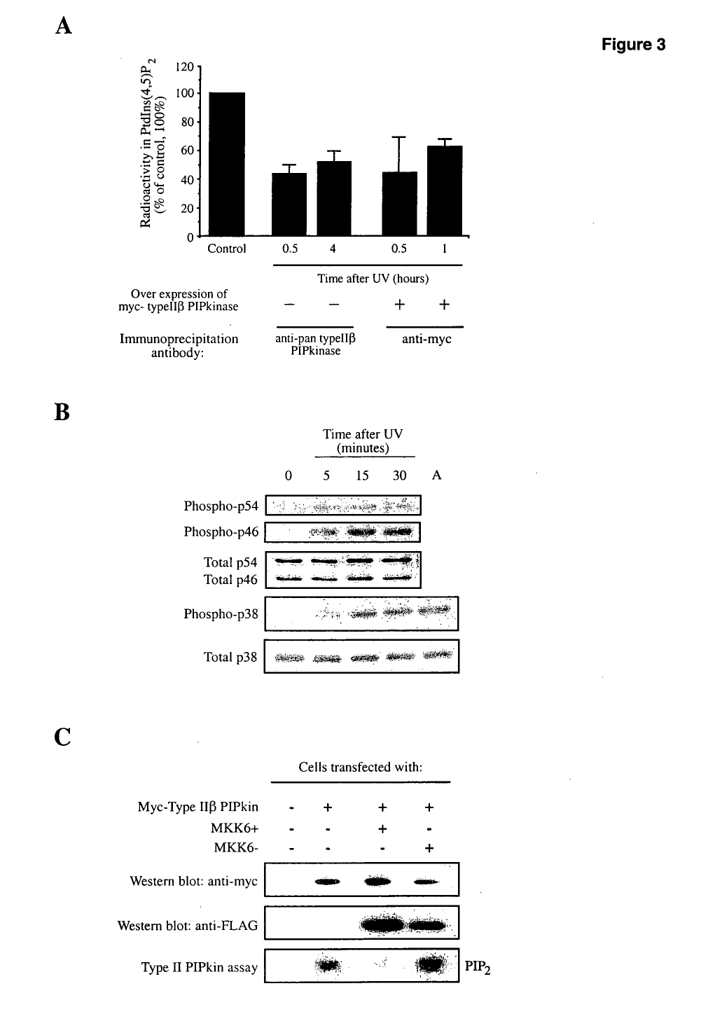

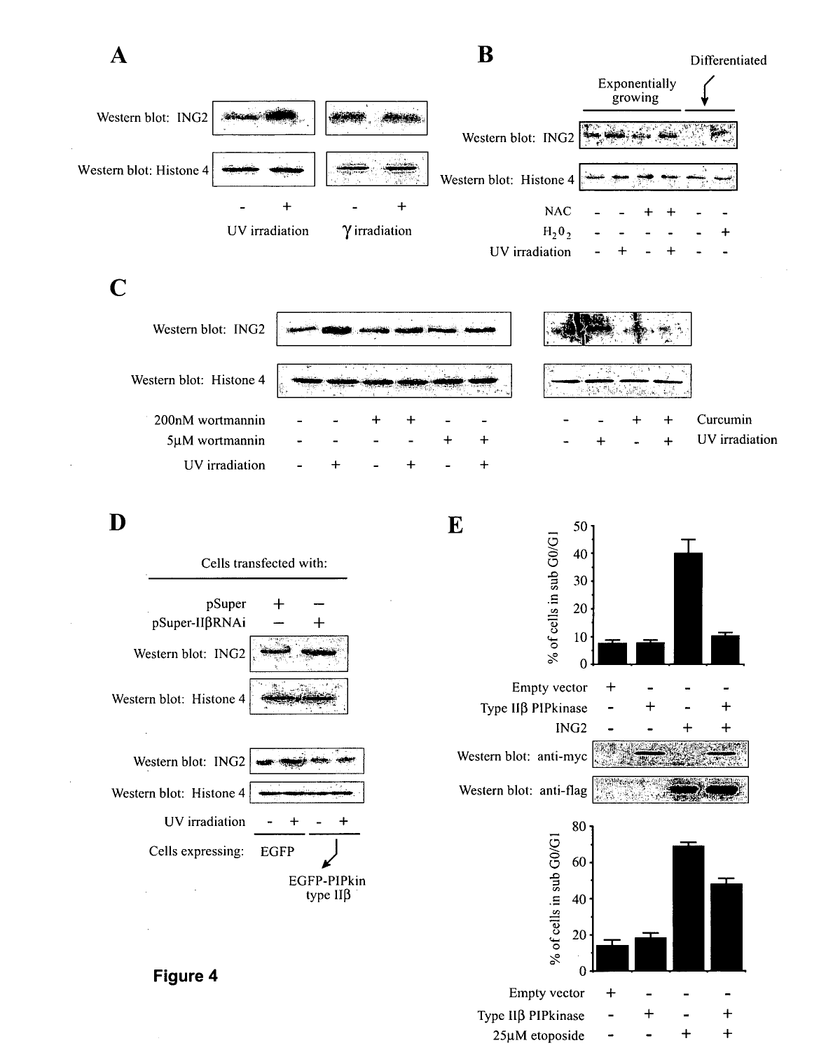

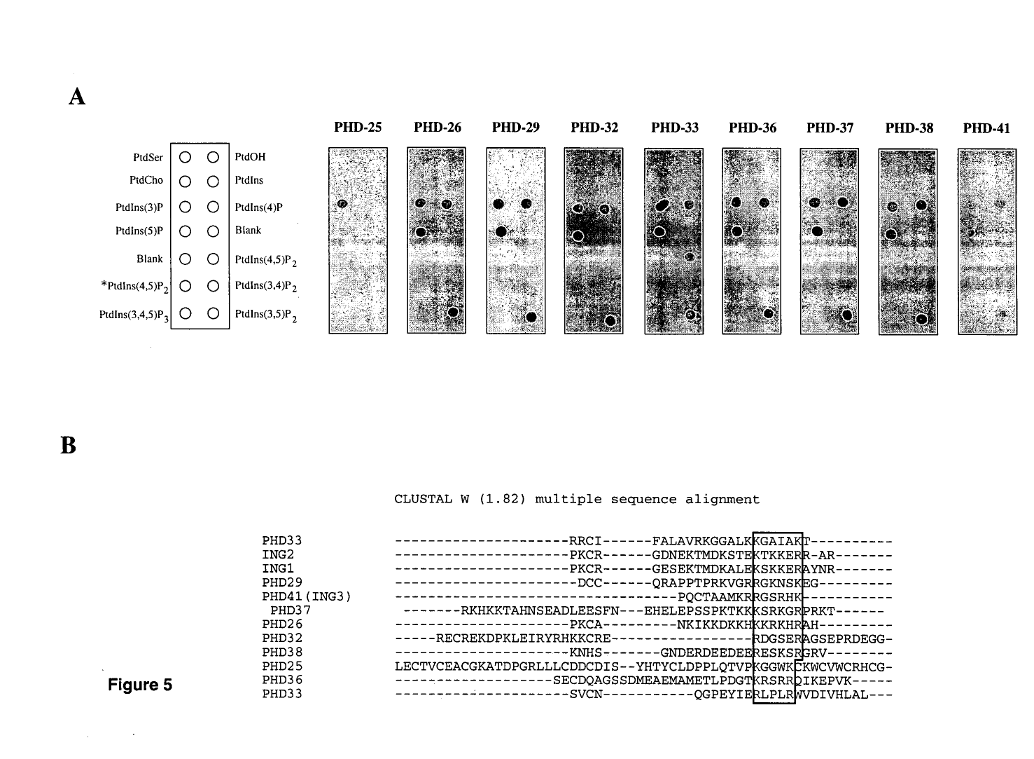

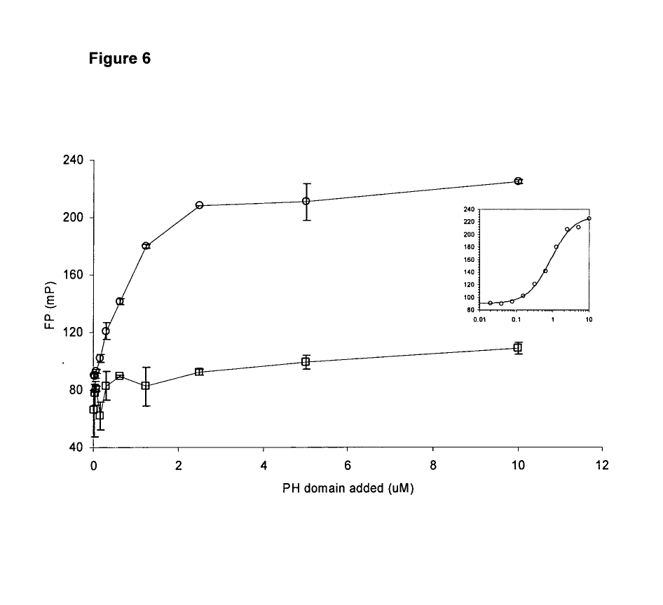

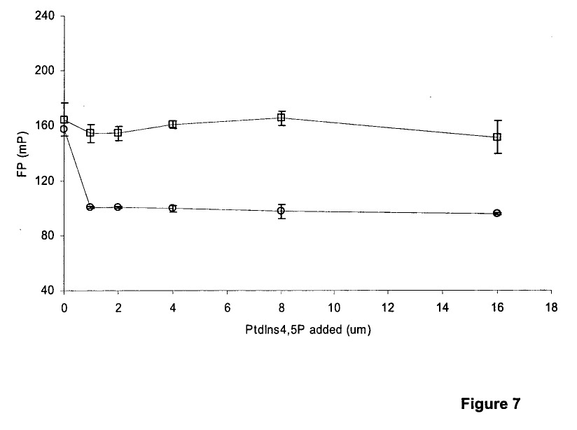

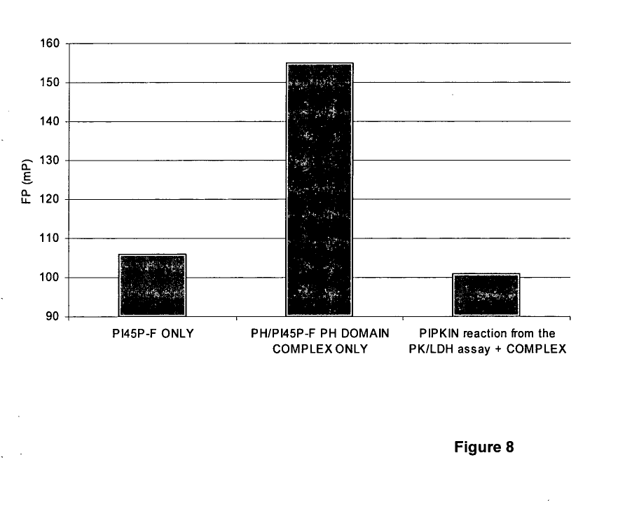

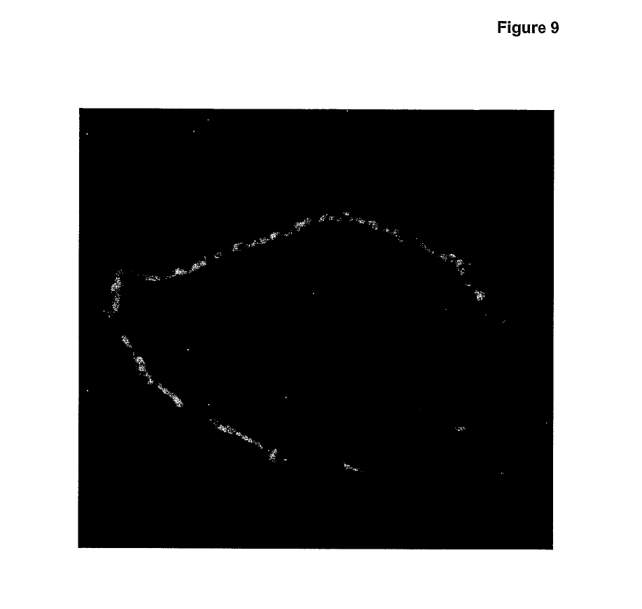

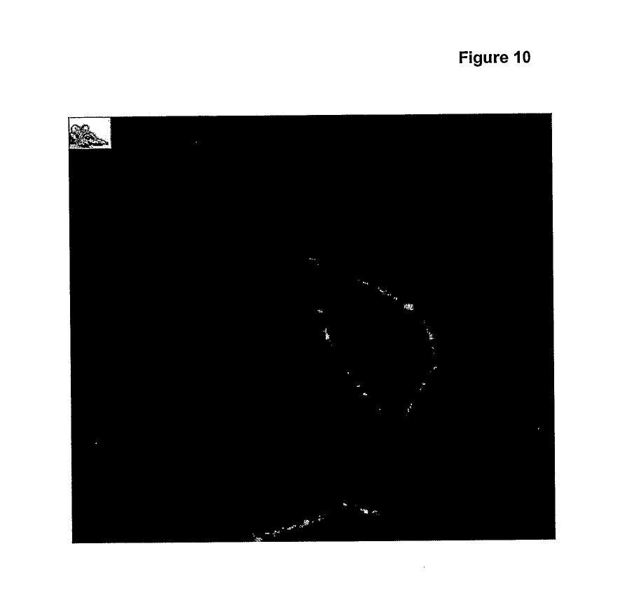

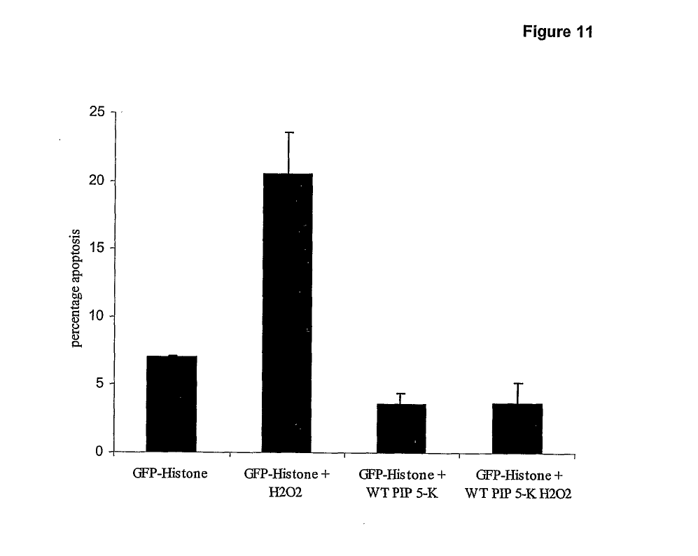

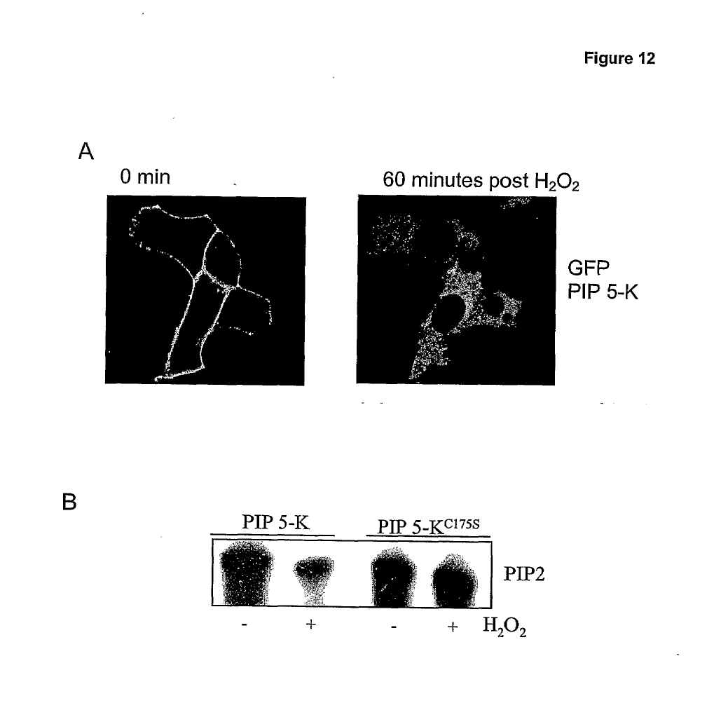

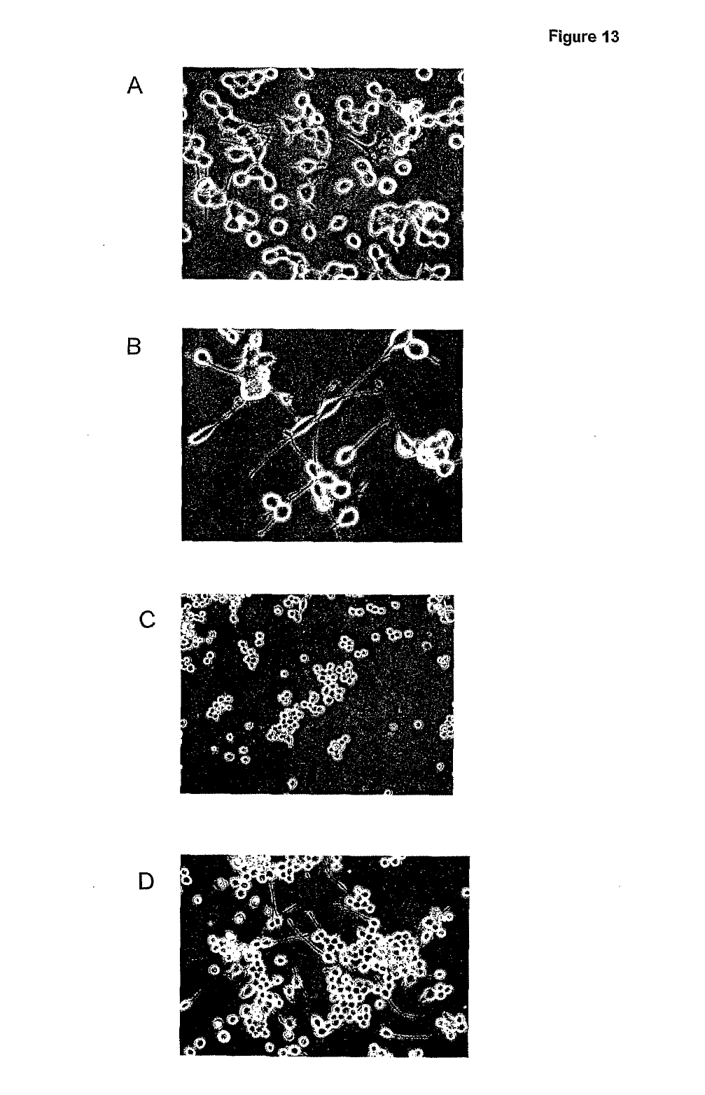

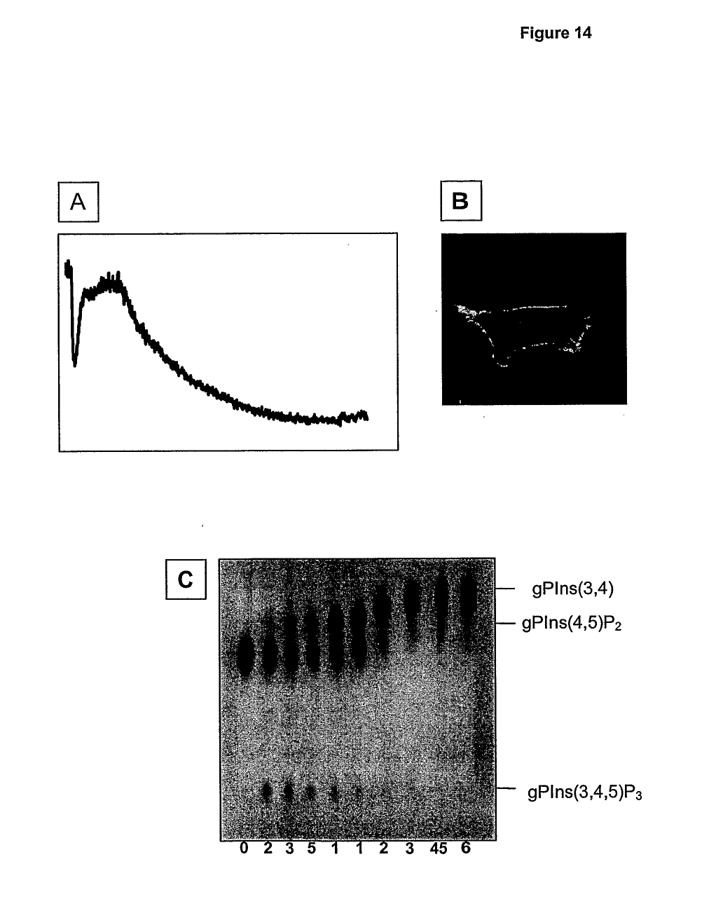

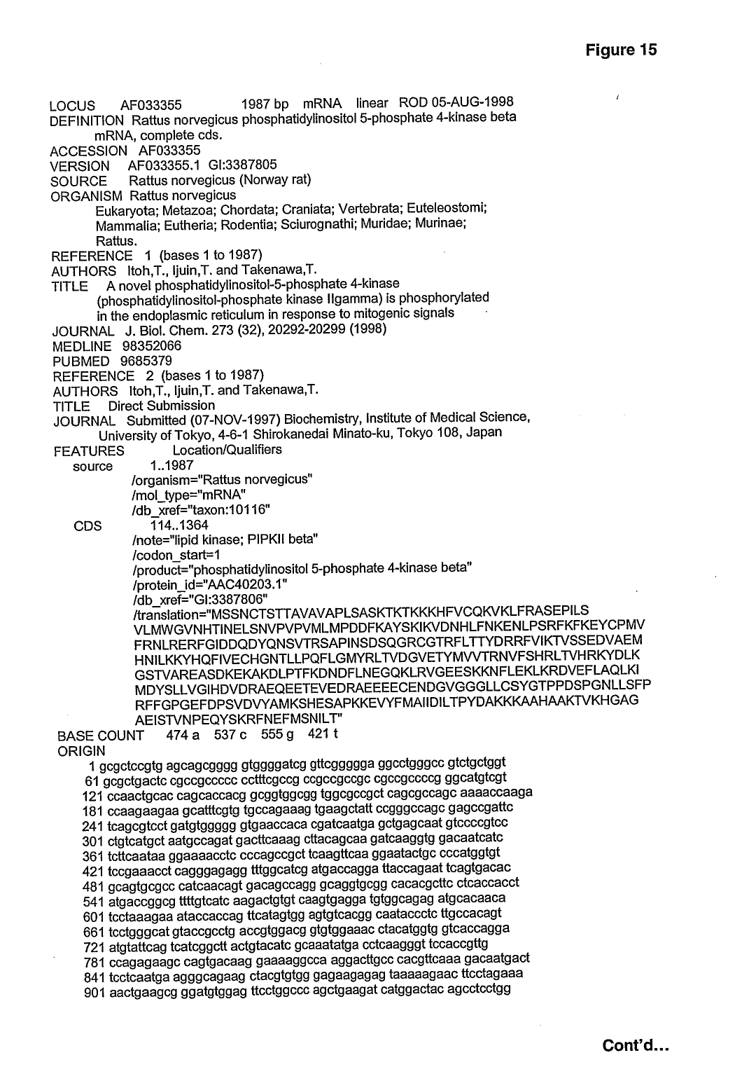

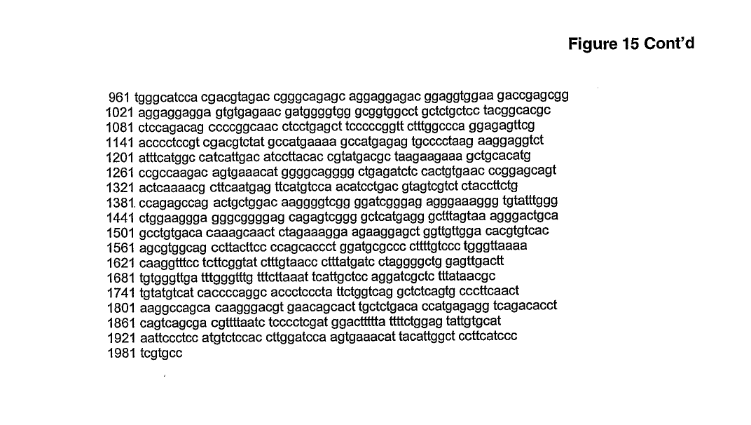

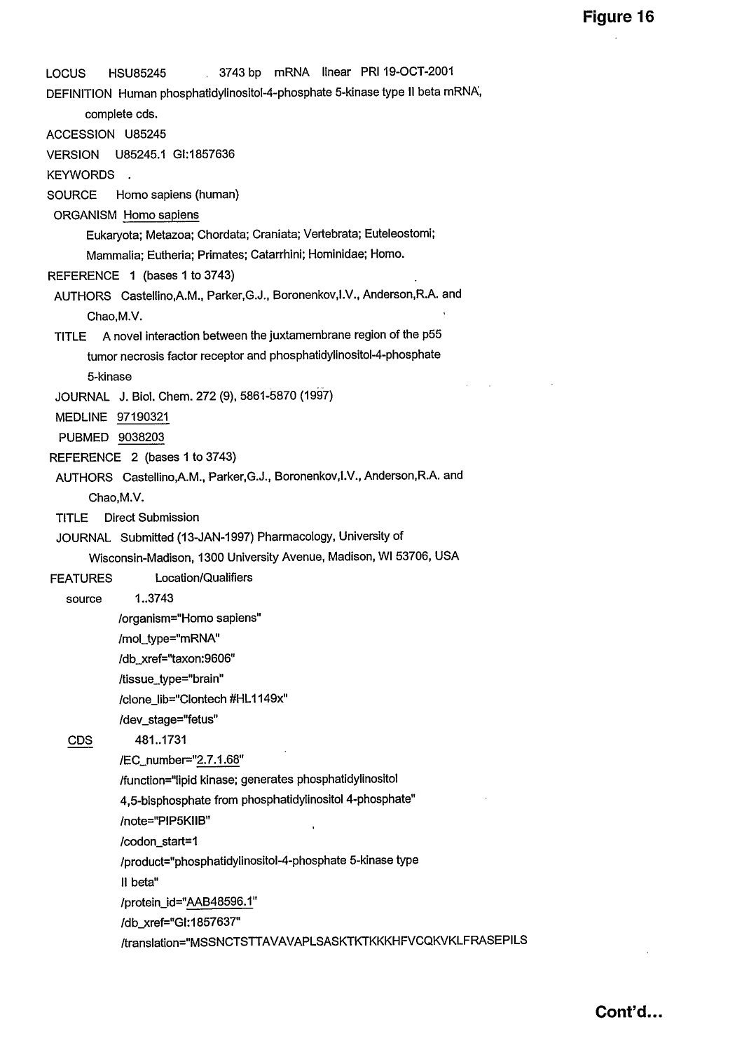

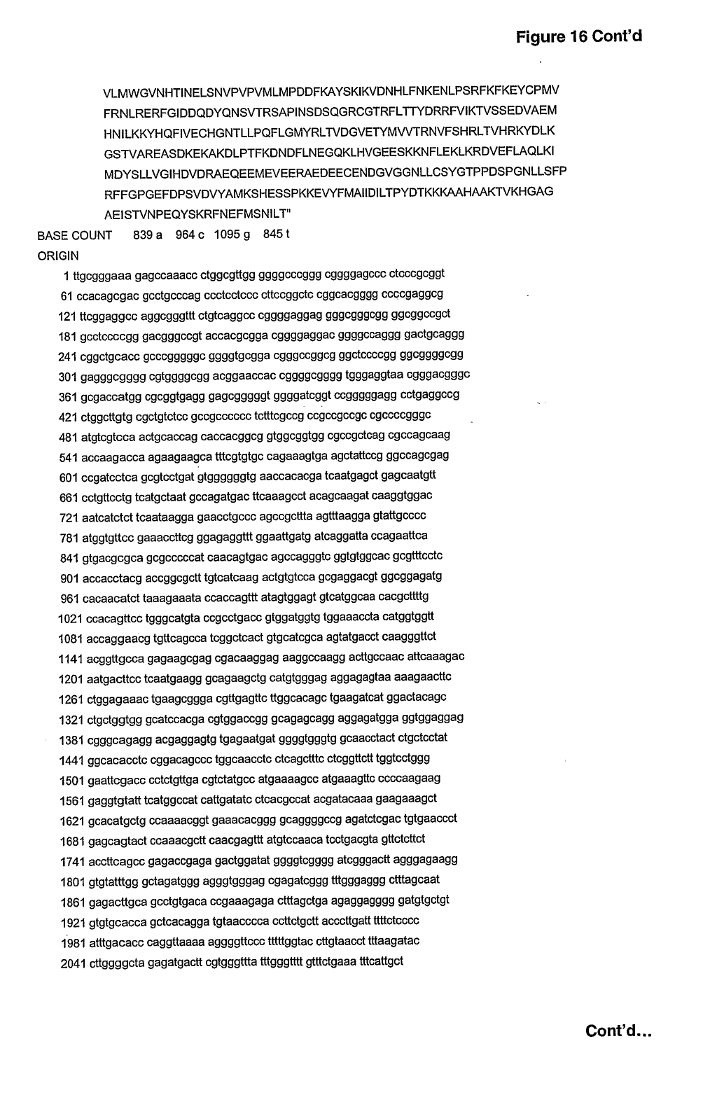

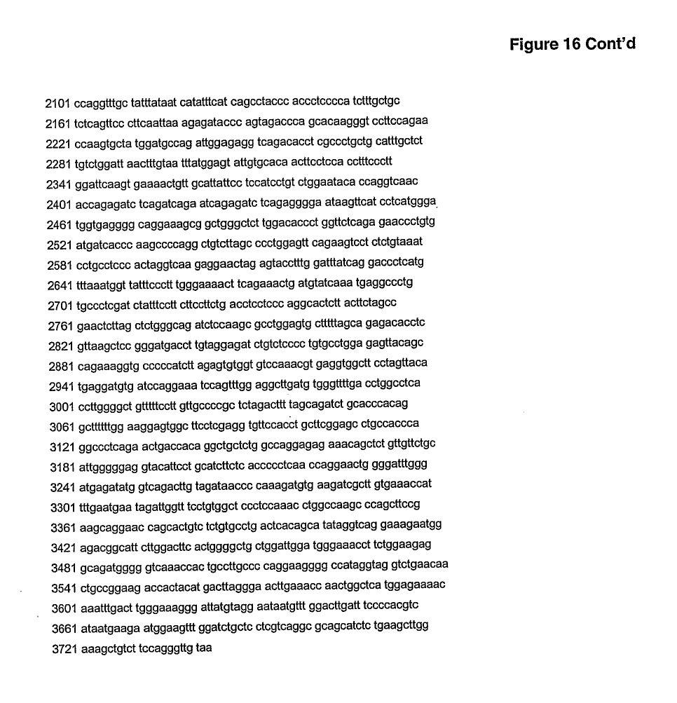

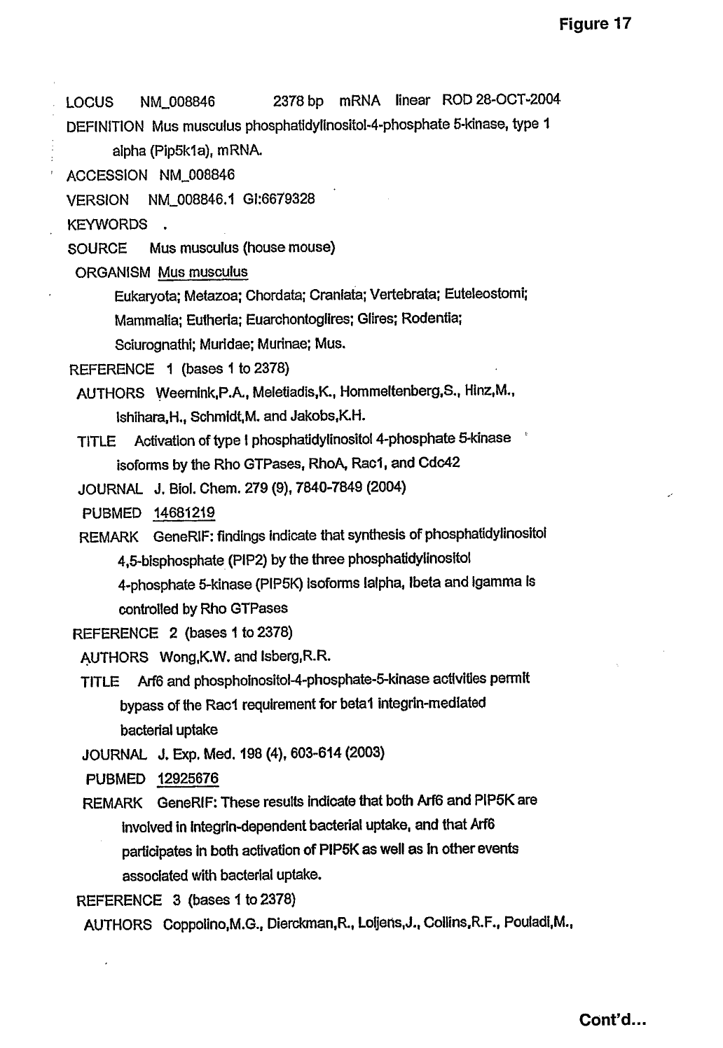

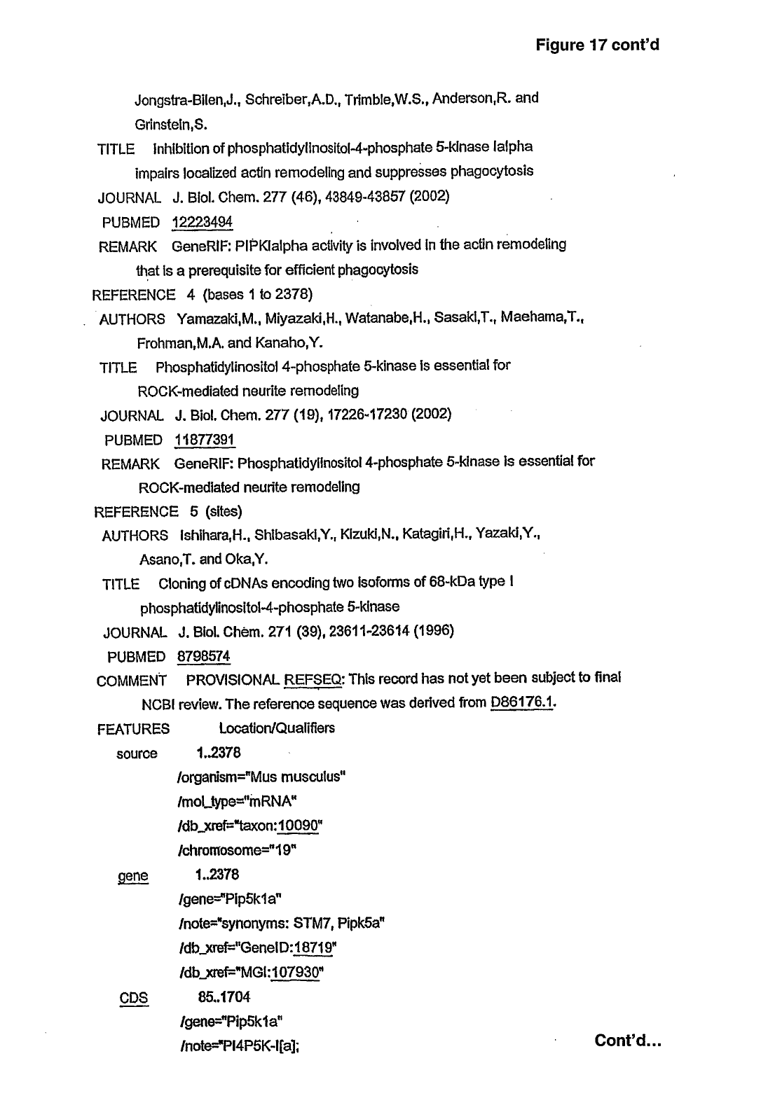

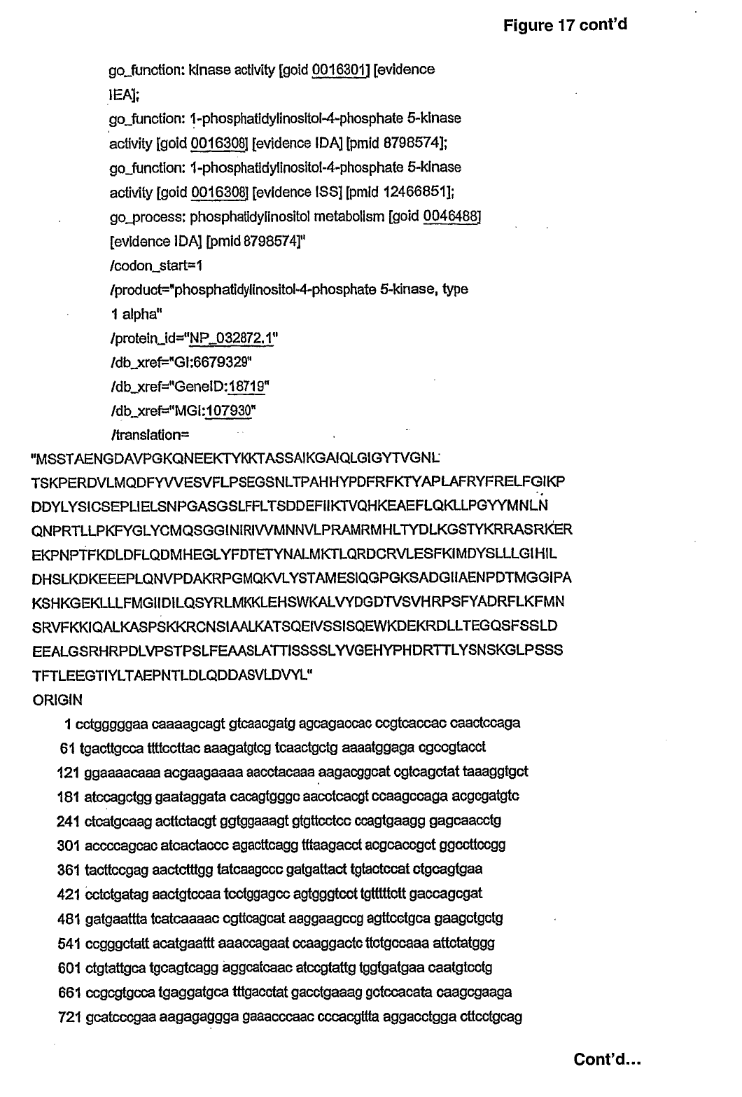

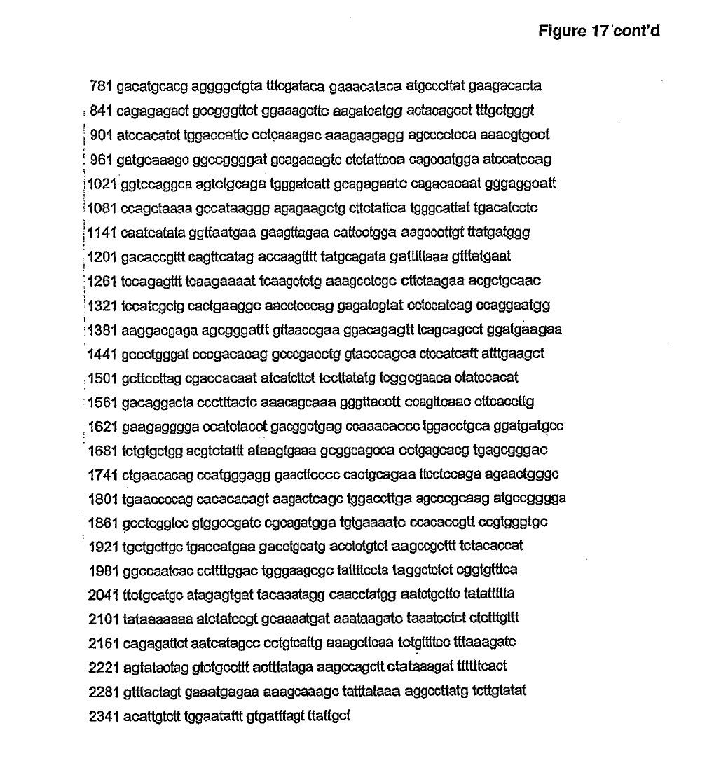

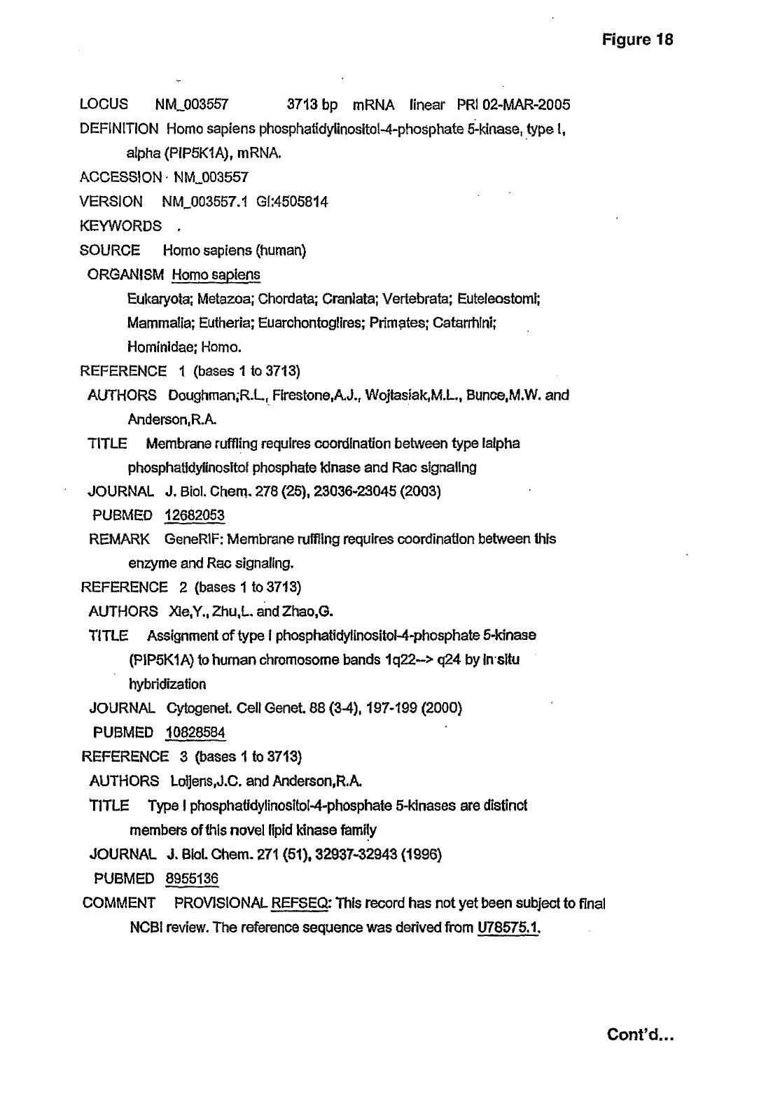

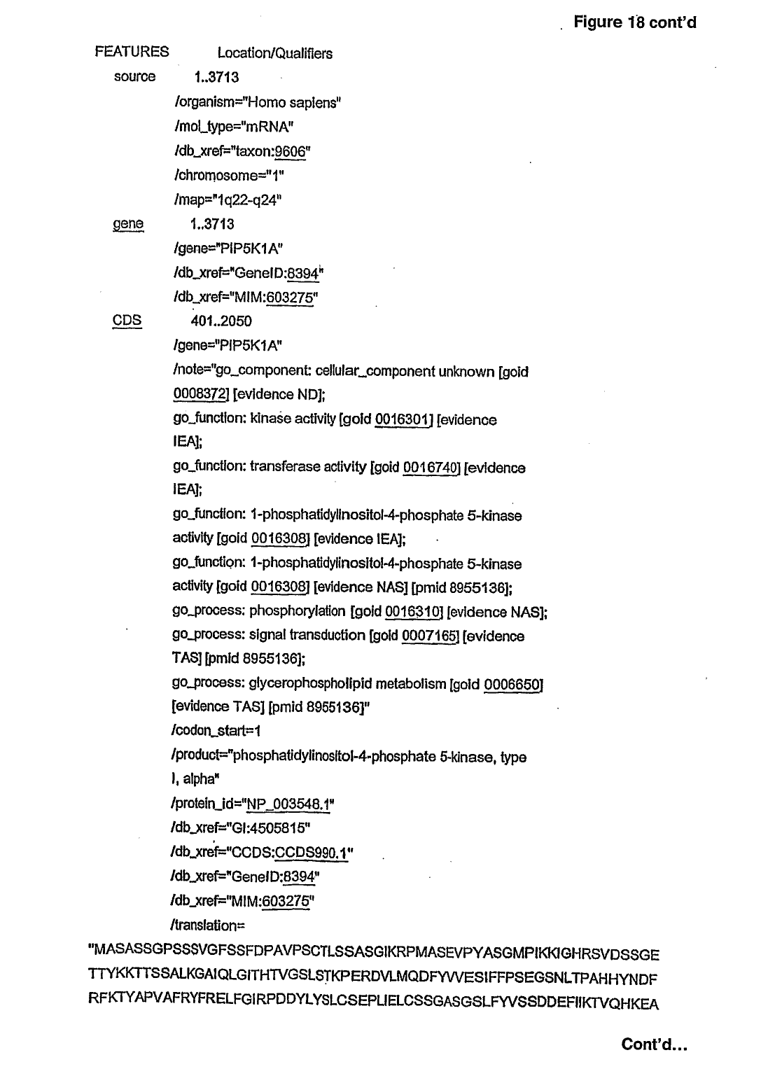

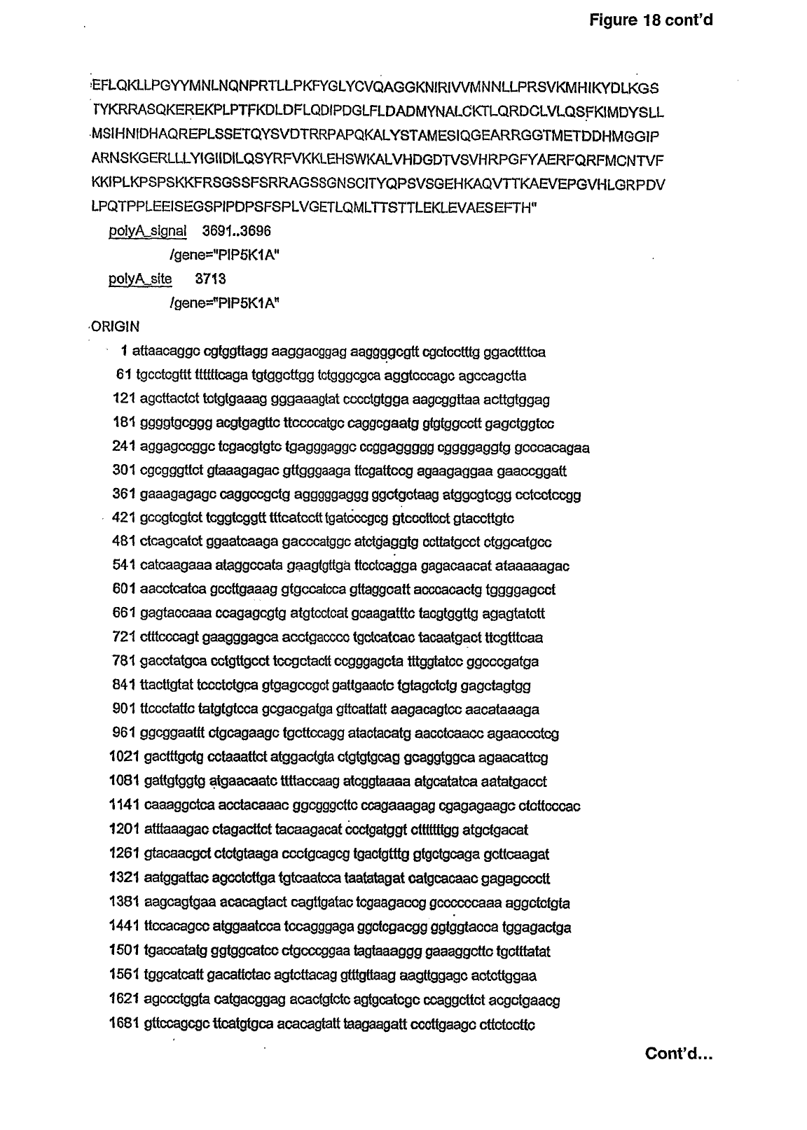

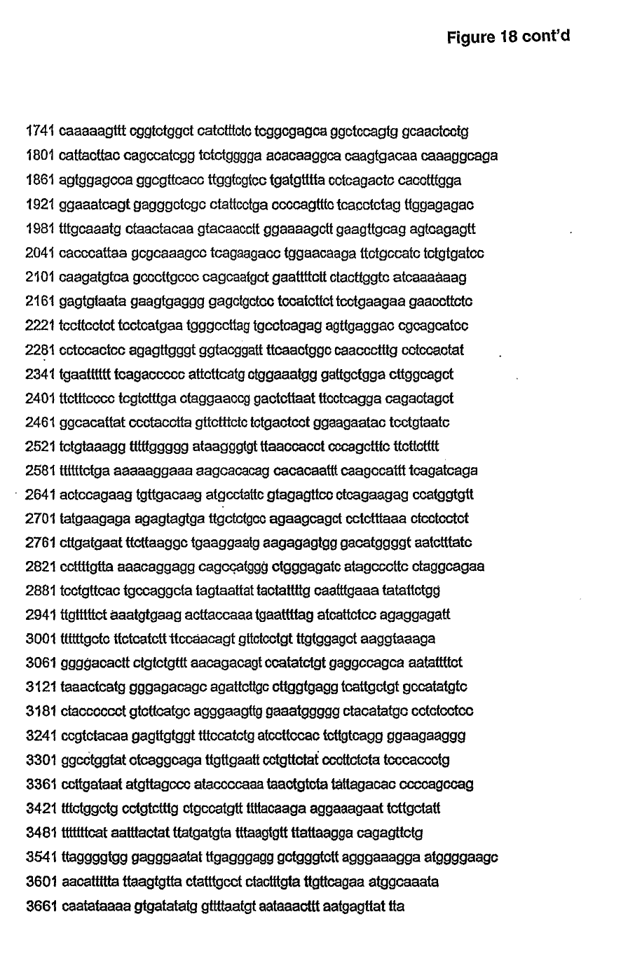

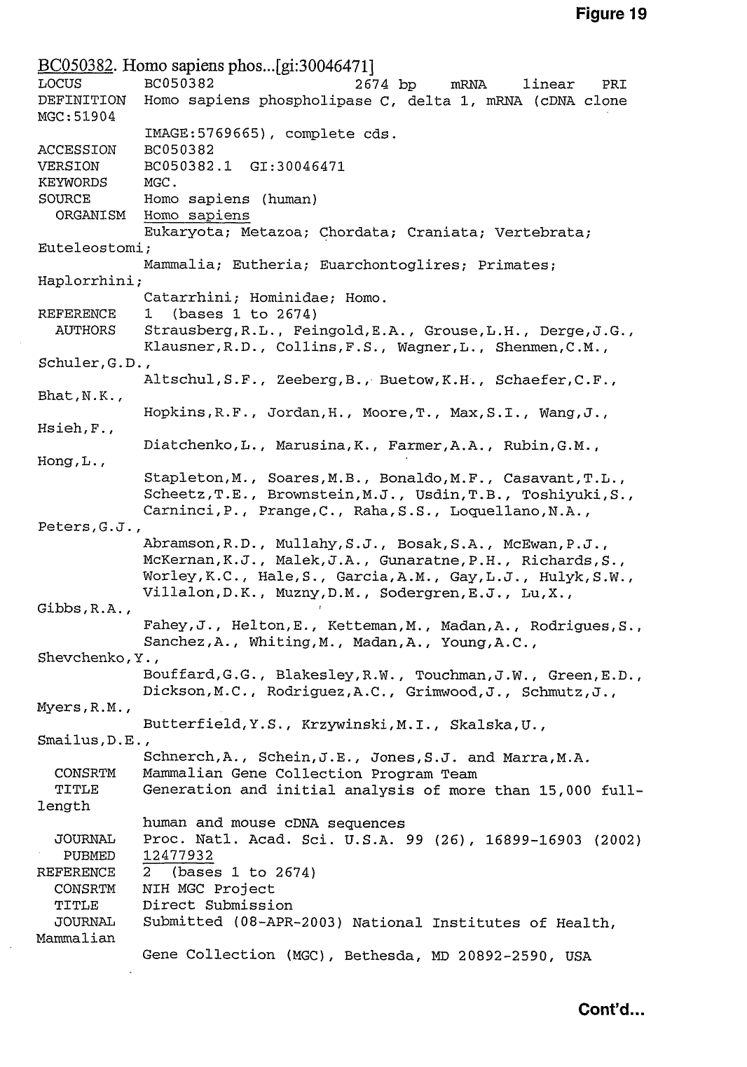

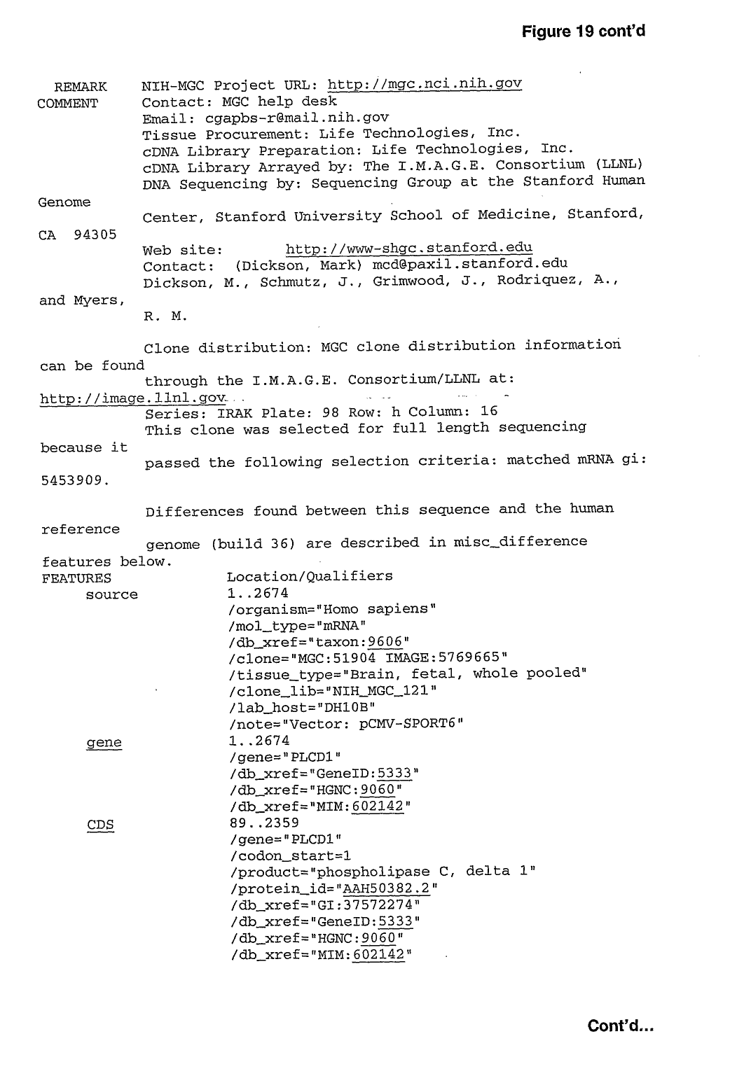

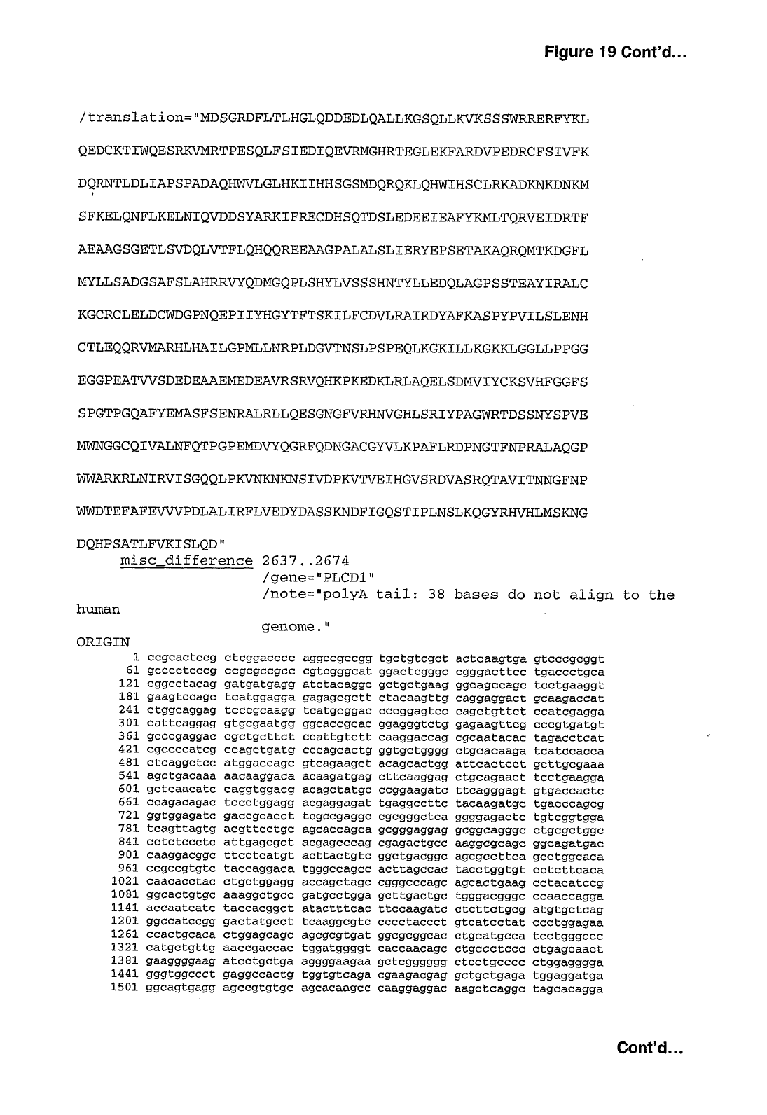

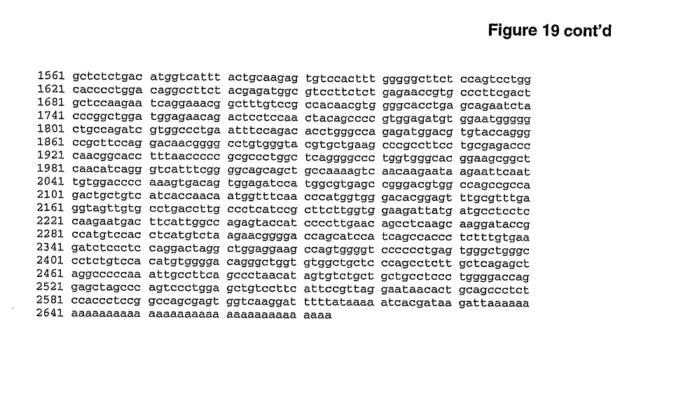

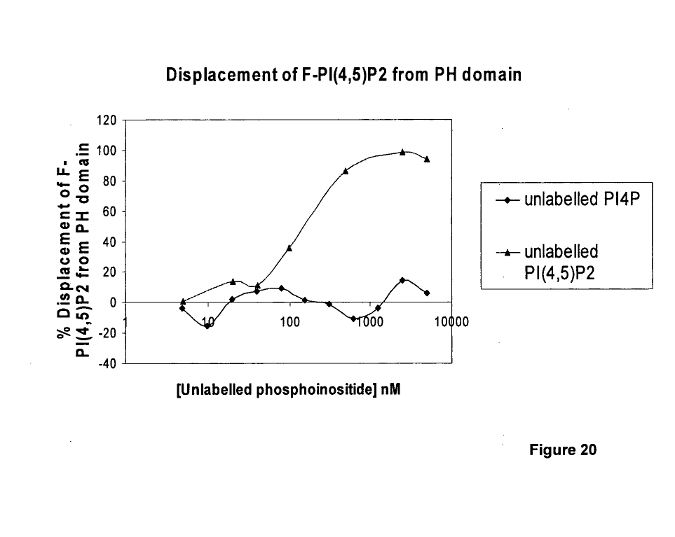

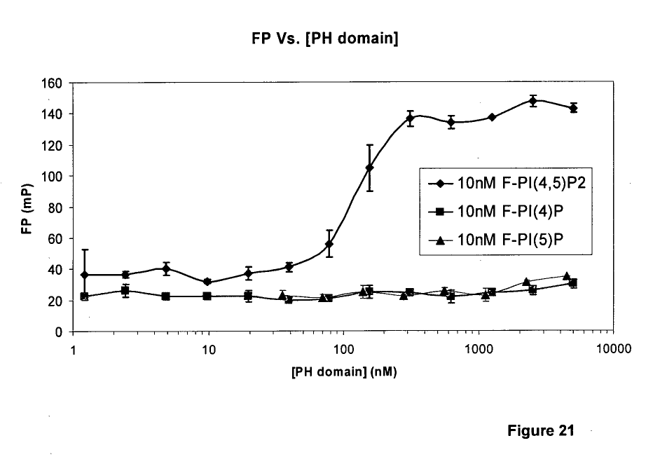

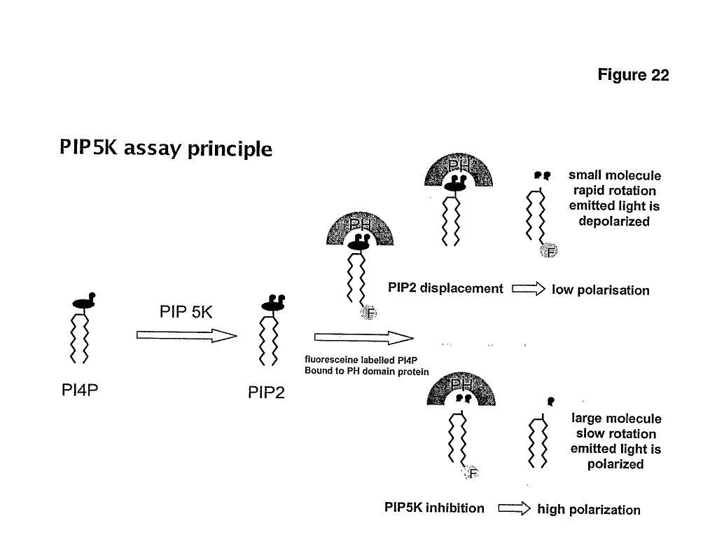

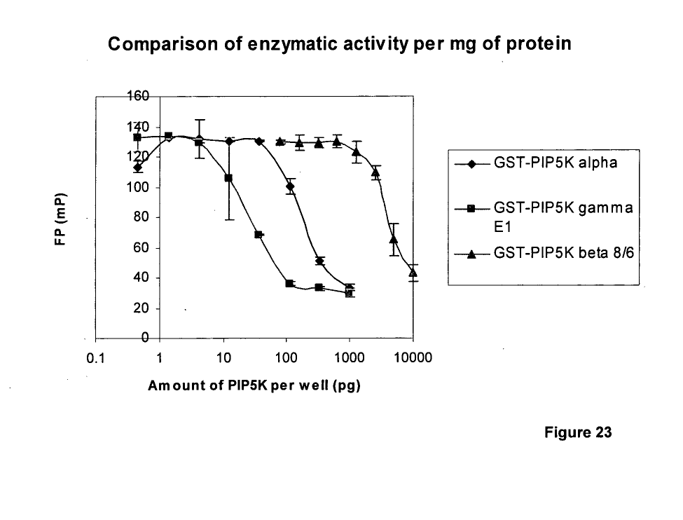

Various forms of irradiation, chemicals such as etoposide and cisplatin, highly reactive free radicals and peroxides, hypoxia and the deprivation of essential nutrients lead to intracellular damage (proteins, lipids and DNA), which can initiate pathways aimed at repairing the damage. Cellular phosphoinositide levels have previously been shown to change as a consequence of osmotic, oxidative and UV damage in mammalian cells and to drought and salt stress in plants. However, no studies have determined a role for nuclear phosphoinositides or defined downstream protein targets mediating phosphoinositide-stress activated pathways. To study changes in nuclear phosphoinositides in response to cellular stress, we used Murine erythroleukaemia (MEL) cells as a model system because preparations of nuclei from these cells have been proven to be of high purity. To visualise changes in phosphoinositides, various independent methods were used. The mass level of both PtdIns4P and PtdIns5P were measured using an assay that relies on the differential in vitro specificities of type I and type II PIP kinases. These analyses showed that both nuclear PtdIns4P and PtdIns5P increased three-fold after treatment with UV irradiation (FIG. 1a). Gamma irradiation (10 Gy) led to a small (approximately 20%) decrease in the PtdIns4P level but no detectable change in the level of PtdIns5P in whole nuclei (FIG. 1a). To determine if nuclear PtdInsPs were regulated by oxidative stress we treated differentiated MEL cells with H₂O₂, BSO (L-buthionine[S,R]-sulphoximine, a glutamyl-cysteine synthetase inhibitor, which increases intracellular reactive oxygen species (ROS)) or NAC. (N-acetyl-cysteine, which increases cellular glutathione levels and cell' capability to deal with ROS). In differentiated MEL cells both H₂O₂ and BSO increased the levels of nuclear PtdIns4P and PtdIns5P (FIG. 1b). In contrast NAC lowered the level of nuclear PtdIns4P and PtdIns5P (FIG. 1b). HPLC analyses of radiolabelled nuclear phospholipids isolated from control cells, cells treated with H₂O₂ and cells irradiated with UV light demonstrated the absence of 3-phosphorylated phosphoinositides (data not shown).

Previous studies demonstrated that nuclear phosphoinositides, PtdIns 4-kinase and PtdIns4P 5-kinase activities are present on the inner nuclear matrix in rat liver and in NIH3T3 cells. To investigate the sub-nuclear localisation of UV-induced nuclear PtdInsPs, we fractionated nuclei to generate nucleoplasm (containing nuclear membranes and soluble nuclear material) and the chromatin enriched fraction (CEF) (FIG. 1c inset). UV irradiation induced increases in PtdIns4P and PtdIns5P levels in the CEF to an extent comparable to that seen in whole nuclei (FIG. 1c). In addition to UV irradiation, etoposide treatment of HT1080 cells also caused an increase in the level of both PtdIns4P and PtdIns5P in the CEF (FIG. 1c). UV irradiation induced activation of the JNK kinase and p38 MAP kinase pathway has been shown to be sensitive to wortmannin. Moreover wortmannin at concentrations above 500 μM is able to inhibit nuclear PtdIns-kinases (our unpublished data and) as well as a number of other protein kinases involved in DNA damage signalling. Curcumin, although having many cellular targets is also able to inhibit PIKfyve, an enzyme capable of generating PtdIns5P from PtdIns. Both of these compounds inhibited the UV light-induced accumulation of PtdIns4P and PtdIns5P (FIG. 1d/1e), while themselves having no effect on either MEL cell viability or the cell cycle profile. Thus, the PtdInsP pool present in the CEF is a target for regulation in response to cellular stress.

Changes in nuclear PtdInsPs may occur via multiple mechanisms including activation of PtdIns-kinase, inhibition/activation of phosphatases or inhibition of phospholipases. PtdIns5P-4-kinases (type II PIP kinases) have been shown in vitro to phosphorylate PtdIns5P, phosphatidylinositol 3 phosphate (PtdIns3P) and phosphatidylinositol 3,5-bisphosphate (PtdIns(3,5)P₂) on the 4′-position, although kinetically they prefer PtdIns5P. There are three isoforms of type II PIP kinases of which α and β are cytoplasmic while β localises both in the cytosol and the nucleus. It is not known which phosphoinositides type II β PIP kinases phosphorylate in vivo (if any) however, the in vitro preference for PtdIns5P and its nuclear localisation may suggest an important physiological role for type II β PIP kinase in the regulation of the nuclear PtdIns5P levels.

MEL cells were transfected to express either EGFP-type II β PIP kinase or EGFP and the level of PtdIns5P in the CEF was measured before and after UV irradiation. As previously demonstrated EGFP-type II β PIP kinase was expressed in MEL cell nuclei (FIG. 2a). The CEF isolated from cells expressing EGFP-type II β PIP kinase showed a small decrease (approximately 15%) in the mass of PtdIns5P compared to cells expressing EGFP. Overexpression of EGFP-type II β PIP kinase completely suppressed the UV-induced increase in nuclear PtdIns5P (FIG. 2c). To define the role of endogenous type II β PIP kinase in the regulation of nuclear PtdIns5P in vivo, we suppressed its expression using RNA interference (RNAi). The expression of endogenous type II β PIP kinase was decreased in nuclei from cells transfected with pSuper-II βRNAi, compared to nuclei from cells transfected with pSuper (FIG. 2b). The mass of PtdIns5P was increased by 73% in the CEF isolated from cells transfected with pSuper-IIβRNAi (FIG. 2c). Furthermore, cells transfected with pSuper-IIβRNAi showed no further increase in the mass of PtdIns5P after UV treatment (data not shown). No changes in the level of PtdIns5P in the cytosol/membrane fraction were detected (data not shown).

The increase in the level of nuclear PtdIns5P induced by RNAi-mediated suppression of type II β PIP kinase suggested that inhibition of type II PIP kinase activity may be one mechanism leading to the accumulation of nuclear PtdIns5P after UV irradiation of MEL cells. To test this hypothesis endogenous type II PIP kinases were immunoprecipitated from control-treated and UV-irradiated MEL cells and their activities were measured using PtdIns5P as a substrate. UV irradiation led to a decrease in endogenous type II PIP kinase activity, which was still observed four hours after irradiation (FIG. 3a). Western blotting demonstrated that the same amount of type II PIP kinase was present in the immunoprecipitates (data not shown). As the antibody used to immunoprecipitate recognises all type II PIP kinase isoforms, we transfected MEL cells with myc-tagged type II β PIP kinase and measured its activity in immunoprecipitates in response to UV irradiation. A similar degree of inhibition of PIP kinase activity was observed at early time points after UV irradiation (FIG. 3a). Western blotting demonstrated that the same amount of type II β PIP kinase was present in the immunoprecipitates (data not shown). UV irradiation is known to regulate both stress-activated protein kinases (SAPKs) and mitogen-activated protein kinases (MAPKs) and therefore we determined if any of these UV induced pathways impinged on type II β PIP kinase activity. UV irradiation induced phosphorylation of p54, p46 (two SAPKs) and p38 (a member of the MAPK family) (FIG. 3b). In addition the p42 and p44 MAPK isoforms were also activated (data not shown). Pre-treatment of MEL cells with SB 203580 (which inhibits the kinase activity of p38) blocked UV light-induced inhibition of type II β PIP kinase activity. This observation suggested that the p38 stress-activated signalling cascade could regulate type II PIP kinase activity in vivo, although it is possible that UV induced PtdIns5P accumulation could lead to product-mediated inhibition of type II β activity through the activation of the p38 pathway. Therefore we assessed whether activation of the p38 pathway alone could mediate type II β PIP kinase inhibition. Thus we overexpressed a constitutively-activate form of MKK6 (mutation of a serine to a glutamic acid, MKK6+), a member of the MAPK kinase family which specifically regulates the activation of the β, β2 and β p38 MAP kinases, or a kinase-inactive form of MKK6 (mutation of a serine to alanine, MKK6−) together with type II β PIP kinase in HEK293 cells. Type II β PIP kinase activity was severely impaired in immunoprecipitates from cells cotransfected with the constitutively-active MKK6 (FIG. 3c). Cotransfection with the kinase-inactive MKK6 had no effect on type II βPIP kinase activity (FIG. 3c). Furthermore treatment of cells with anisomycin, a potent regulator of p38, led to an inhibition (approximately 50%) of type II β PIP kinase activity. These results indicate that cellular stress-mediated activation of the p38 MAP kinase pathway can regulate type II β PIP kinase activity in vivo to regulate the level of nuclear PtdIns5P.

### Example 2

The PHD finger of p33ING2/INGL binds to phosphomonoinositdes (Gozani et al). ING2 has been shown to be nuclear, suggested to regulate gene transcription in response to stress and therefore represents a good candidate as an in vivo sensor of nuclear PtdInsPs. CEF isolated from MEL cells before and after treatment with various cellular stresses that do (UV and oxidative stress) or do not (gamma irradiation) increase the levels of nuclear PtdInsPs were probed for the presence of endogenous ING2. UV irradiation led to an increased level of endogenous ING2 associated with the CEF (FIG. 4a), whilst gamma irradiation did not (FIG. 4a). Although some UV-induced responses are dependent on an increase in cellular reactive oxygen species, pre-treatment of cells with NAC did not block UV-mediated translocation of ING2 (FIG. 4b). Treatment of differentiated cells with H₂O₂, which causes an increase in nuclear PtdIns4P and PtdIns5P (FIG. 1b), also stimulated ING2 translocation to the CEF (FIG. 4b). Both wortmannin and curcumin, which block PtdIns4P and PtdIns5P synthesis in response to UV irradiation (FIG. 1d/1e), completely inhibited UV-induced translocation of endogenous ING2 to the CEF (FIG. 4c). To further demonstrate that changes in nuclear phosphoinositides regulate ING2 translocation, we isolated CEFs from cells either transfected with pSuper-IIβRNAi or pEGFP-type IIβPIP kinase and probed them for the presence of ING2. Transfection with pSuper-IIβRNAi led to a constitutive translocation of ING2 to the CEF in the absence of UV irradiation, in accordance with pSuper-IIβRNAi mediated increases in the mass of nuclear PtdIns5P (FIG. 4d). Overexpression of type II β PIP kinase completely blocked the UV-mediated translocation of ING2 (FIG. 4d). These data provide compelling evidence that type II β PIP kinase regulates a pool of nuclear PtdIns5P in response to UV irradiation which affects the subnuclear localisation of ING2. The ING family of proteins, including ING2, have been implicated in the regulation of p53 function. In order to demonstrate a direct role for type II β PIP kinase in the modulation of ING2 function, we chose HT1080 cells as they have a functional p53 pathway. ING2 led to cell death when over-expressed alone in HT1080 cells (FIG. 4e). Type II β PIP kinase when expressed alone had no effect on cell death nor on gross cell morphology. However, when the two proteins were over-expressed simultaneously, cell death induced by ING2 was reduced by over 90% compared to over-expression of ING2 alone (FIG. 4e). This clearly indicates that type II β PIP kinase can modulate ING2 function probably by regulating the level of nuclear PtdIns5P. Cell death induced by ING2 has been suggested to occur through the regulation of p53 acetylation. Moreover a mutant of ING2 unable to interact with phosphomonoinositides was unable to induce p53 acetylation (Gozani et al.). Etoposide treatment of HT1080 cells leads to increases in nuclear PtdIns5P (FIG. 1c) and cell death which is dependent on p53 and ING2 function (Gozani et al.). In accordance, we found that cell death induced by etoposide was partially inhibited by overexpression of type II β PIP kinase (FIG. 4e). Interestingly, type II p PIP kinase also partly inhibited UV irradiation-induced death in MEL cells (control EGFP cells 22+/−2%, EGFP-PIPkin type II β expressing cells 15.1+/−2 at 50 J/m), whereas RNAi-mediated suppression of endogenous type II β PIP kinase enhanced cell death in response to UV irradiation (control cells 11.1+/−0.9%, pSuper-IIβRNAi 19.7+/−1.5% at 50 J/m). As MEL cells are negative for p53 function and ING2 overexpression in these cells does not induce or enhance UV irradiation-mediated cell death it is likely that there are other downstream proapoptotic targets for nuclear PtdIns5P.

In order to assess the generality of PHD finger interaction with phosphoinositides, we cloned, expressed as GST fusion proteins and tested for phosphoinositide binding in vitro, sixty murine PHD fingers. Ten of the PHD fingers bound strongly to phosphoinositides (FIG. 5a) including ING1, 2, 3 and three unknown putative nuclear proteins. PHD33 is PHF6, a candidate gene for Borjeson-forssman-lehmann syndrome; PHD26 is TAF(II)140, a component of the TFIID complex; PHD32 is CGBP, a CpG binding protein; PHD25 is MLL3-like protein, which contains five putative PHD fingers, one HMG and a SET (Suppressor of variegation, Enhancer of zeste, Trithorax) domain, a putative methyltransferase. Sequence alignment of these PHD domains showed that all the strong binders contained a patch of lysine/arginine residues at the C-terminal extension (FIG. 5b). A mutational analysis of ING2 (Gozani et al.) demonstrates that the C-terminal basic patch is essential for phosphoinositide binding. All of the PHD fingers interacted with PtdIns(3)P, PtdIns4P, PtdIns5P and PtdIns(3,5)P₂, with the exception of PHD25, which interacted most strongly with PtdIns(3)P. Interestingly, other studies have demonstrated changes in nuclear PtdIns(3)P during the G2/M transition, after treatment with all trans-retinoic acid (HL-60 cells) and during compensatory liver growth after partial hepatectomy. We expect that diverse and contextual modulation of PtdIns(3)P, PtdIns4P and PtdIns5P within the nucleus will differentially regulate PHD containing protein functions.

We show that the level of nuclear PtdIns4P and PtdIns5P increase in response to cellular stress and that these PtdInsPs can act as ligands for a number of PHD finger containing proteins. This study uncovers, for the first time, a direct function for type II β PIP kinase in regulating stress-induced nuclear phosphoinositide changes, which impinge on proapoptotic pathways through the modulation of ING2 function. The demonstration that type II β PIP kinase activity is regulated by the p38 MAP kinase pathway suggests that signalling induced by other mechanisms such as cytokines (IL-1/TNF) are likely to impinge on nuclear phosphoinositides and PHD-containing protein function.

As our PHD screen has identified proteins, which control chromatin organization by diverse mechanisms (PHD 25, 26, 33), we speculate that changes in nuclear PtdInsP levels might trigger a coordinated nuclear response by establishing a specific and contextual chromatin state which may impinge on the complex pathways controlling cell arrest and cell death.

### Example 3

### Regulation of Cellular PtdIns(4,5)P₂ Levels

Changes in intracellular PtdIns(4,5)P2 levels following stimulation with neurokinin A were visualised in real time using a fusion protein of GFP and the PH domain of PLCdelta1 as a probe. GFP PLC delta 1 is a highly specific in vivo probe for PIP2 in cells.

HeLa cells were transiently transfected with an expression plasmid encoding a fusion protein of GFP and the PH domain of PLCdelta1. PtdIns(4,5)P₂ levels were monitored before, during and after stimulation with neurokinin A. Results are shown in FIG. 9.

This experiment shows that growth factors such as neurokinin A stimulate PtdIns(4,5) P₂ depletion, which can be visualised in real time with our PtdIns(4,5)P₂ probe. Cells quickly recover their PtdIns(4,5) P₂ levels after growth factor stimulation prompting the return of the probe back to the plasma membrane. Healthy cells tightly manage their PtdIns(4,5)P₂ levels.

### Example 4

### Effect of H₂O₂ Induced Apoptosis on PtdIns(4,5)P₂ Levels

HeLa cells were transiently transfected with an expression plasmid encoding a fusion protein of GFP and the pleckstrin homology domain of PLCdelta1. GFP PLC delta1 is a highly specific in vivo probe for PtdIns(4,5)P₂ in cells. Results are in FIG. 10.

Panel A: In healthy cells with high levels of PtdIns(4,5) P₂ the probe remains at the plasma membrane.

Panel B: The picture shows that after cells are stimulated with an apoptotic concentration of H₂O₂ (600 microMolar). The probe rapidly translocates from the plasma membrane into the cytosol. The translocation is sustained as we see no recovery of the probe back to the plasma membrane inferring that PtdIns(4,5) P₂ levels are still low hours after stimulation. This experiment shows that apoptotic concentrations of H₂O₂ trigger sustained depletion of PtdIns(4,5) P₂ levels and cells cannot recover their PtdIns(4,5) P₂ levels.

Thus H₂O₂ sends cells into apoptosis by depleting PtdIn(4,5)P₂ levels within cells.

### Example 5

### Role of Type 1 PIP Kinase in Apoptosis

The inventors have shown that type I PIP kinase protects cells from ROS induced apoptosis. HeLa cells were transiently transfected with expression plasmids encoding for the listed proteins. Cells were then treated with 600 microMolar H₂O₂ for 24 hours. All cells were collected and examined for apoptosis. Apoptosis was determined by examining nuclear morphology of green cells (GFP Histone) after staining with a nuclear stain. Cells with fragmented nuclei were scored positive for apoptosis. This experiment shows that 600 microMolar H₂O₂ induces apoptosis in HeLa cells. Ectopic expression of PIP 5-K protects HeLa cells from apoptosis after H₂O₂ treatment. Results are in FIG. 11.

The inventors then showed that ROS target and negatively regulate type 1 PIP kinase. During ROS induced apoptosis H₂O₂ targets and inactivates type I PIP kinase. Results are shown in FIG. 12.

HeLa cells were transiently transfected with expression plasmids encoding for a GFP fusion protein of PIP 5-K. In healthy cells GFP PIP 5-K is located at the plasma membrane. Cells were then treated with 600 microMolar H₂O₂ and the localisation of the GFP PIP 5-K was monitored on line using confocal laser scanning microscopy. This experiment shows that 600 microMolar H₂O₂ induces translocation of GFP PIP 5-K away from the plasma membrane. H₂O₂ regulates the localisation of the enzyme in vivo. (FIG. 12, panel A)

Recombinant wild type PIP 5-K and a point mutation of PIP 5-K-(cysteine 175 altered to a serine residue) were both treated with H₂O₂ and enzyme activity was assayed in vitro by measuring the amount of product that is produced after incubation with substrate and radioactive ATP. The products were then analysed on TLC plate using phosphoimager. This experiment shows that PIP 5-K activity is sensitive to treatment with apoptotic concentrations of H₂O₂ whereas Cysteine 175 is not. (FIG. 12, panel B).

Thus the experiment allows us to conclude that PIP 5-K is negatively regulated by H₂O₂. Firstly, the enzyme is physically removed from its in vivo substrate and secondly H₂O₂ inactivates the activity of the enzyme probably by targeting a distinct cysteine residue on the protein. A likely target for inactivation is cysteine 175.

### Example 6

### Role of PIP5K Alpha in Neurite Outgrowth and Cell Rounding

Neuroblastoma cells (N2A) were transfected either with a control plasmid (GFP) or with a kinase inactive mutant of PIP 5-K alpha (which can no longer elevate PtdIns(4,5) P₂ levels) in the presence of serum. 24 hours after transfection morphology was recorded using conventional phase contrast microscopy (FIG. 13, panels A & B). This experiment shows that neurite like structures are clearly evident in the kinase dead cells. Kinase dead PIP 5-K can induce neurite like structures in neuroblastoma cells in the presence of serum.

This experiment is a fore runner of the experiment in panel C and D. The general concept here is that PIP 5-K alpha activity blocks neurite formation and induces cell rounding. In panel C and D we have used RNAi to knock out PIP 5-K alpha activity.

Lowering PtdIns(4,5) P₂ production causes neurite outgrowth in neuroblastoma cells.

### Example 7

### Activity Assay for Type I (PIP5K) and Type II (PIP4K) PIP Kinases (PIPKINs)

A number of methods were trialled in order to measure the activity of PIPKIN with an assay suitable for high throughput screening (HTS). Eventually a displacement assay was designed to measure the product of the PIPKIN reaction, PtdIns(4,5) P₂. It is based on an observation that the pleckstrin homology (PH) domain of a PtdIns processing enzyme, phospholipase C delta binds strongly to PtdIns(4,5)P₂, the product of the PIPKIN reaction. The PH domain was also chosen for assay development as it is reported to have a high affinity for PtdIns(4,5)P₂ (product) over PtdIns5P or PtdIns4P (the substrates).

The displacement assay relies upon the pre-formation of a complex of a fluorescently tagged product analogue (PtdIns(4,5)P₂—F) bound to the PH domain of phospholipase C. This complex has a high fluorescence polarisation value as it is a large molecule. When this mixture is added to a completed PIPKIN reaction, unlabelled PtdIns(4,5) P₂ produced by the PIPKIN from it's substrate competes with and displaces the bound PtdIns(4,5)P₂—F from the PH domain. Displaced unbound PtdIns(4,5) P₂—F exhibits a low FP value. This decrease in the FP measured for the PtdIns(4,5) P₂—F is proportional to the amount of PtdIns(4,5) P₂ produced in the PIPKIN reaction and therefore proportional to the PIPKIN activity.

A diagrammatic representation of the fluorescence polarisation assay is given in FIG. 22 (the substrate shown is PtdIns4P and the PIPKIN is type I PIP kinase but the assay principle is also applicable to type II PIP kinase (PIP4K) and it's substrate PtdIns5P. All known isoforms of type I PIP kinase (PIP5K) and type II PIP kinase (PIP4K) can also be used in this assay.

The principle of the assay was proven by selectively binding PtdIns(4,5) P₂—F to the PH domain, a fluorescent substrate analogue PtdIns5P—F does not bind (FIG. 6).

The PH domain binds the fluorescent product, being 50% bound at ˜850 nM (binding curve fit inset), whilst 10 μM PH domain shows minimal binding to the fluorescent substrate.

A further study was carried out to show that the PH domain selectively binds to F—PI(4,5) P₂, and not to either of the substrates, F—PI(4)P or F—PI(5)P. Results are in FIG. 21.

This preformed PH domain-PtdIns(4,5) P₂—F complex can be displaced by unlabelled PtdIns(4,5) P₂ product and not by unlabeled substrate and hence the complex does act as a selective sensor for PIPKIN enzyme activity (FIG. 7 and FIG. 20)

It was ascertained during the assay development that the PIPKIN enzyme is much more active in assay systems that contain an ATP regenerating system and we infer from this that the enzyme is product inhibited by ADP. The inventors have shown that a reaction mix containing PIP4K and PtdIns5P was capable of generating enough product to displace a preformed PH domain PtdIns(4,5) P₂—F complex (FIG. 8). This suggests that the assay is suitable for development towards HTS.

The assay was carried out for each of the human PIP5K isoforms, PIP5Kalpha, PIP5K gamma, and PIP5K beta. As shown in FIG. 23, increasing enzyme activity was represented in falling FP values.

### Assay Protocol

A detailed assay protocol is provided below. The protocol is given for type I PIP kinase, but may be used type I or type II PIP kinase, with substitution of appropriate substrate.

### Materials

### Apparatus:

Plates are read on an Analyst HTS plate reader with the following settings:
Z-height=2 mm, Lamp=Continuous, Raw data units=RFU, Reads per well=1, Integration time=100,000 μs, PMT=Digital, Microplate format=Corning 384 low volume, G-factor=0.95.

### Glassware/Plasticware:

Black, Low volume, 384-well non-binding surface plates (Corning, Product code 3676).
Corning black polystyrene non-binding surface 384 well 900 microplate (Product code 3654).

### Chemicals:

2.5× Assay buffer=50 mM HEPES pH7.5 at 25° C.+12.5 mM MgCl₂+1.25 mM EGTA+2.5 mM DTT.
In-house GST-tagged human PIP5KI_{β} enzyme cloned and over-expressed in Baculovirus by Leon Pang and purified by Alex Boakes. Stock at 11.3 μg/ml in GST elution buffer+5% glycerol and stored at −80° C.
GST-tagged PLCδ1 Pleckstrin homology (PH) domain protein expressed and purified in-house in E. Coli by Cameron Bell. Stock in PBS+20% glycerol and stored at −80° C.
BODIPY® PtdIns (4,5)P₂ (Echelon Biosciences, Product code C-45F6). Stock concentration=10 μM, stored at −80° C.
D-myo-Phosphatidylinositol 4-phosphate (PtdIns(4)P) (Echelon Biosciences, Product code P-4008). Stock concentration=1 mM in miliQ H₂O, stored at −20° C.
Adenosine 5′-triphosphate disodium salt (Sigma, Product code A7699).

### Method

2.5× Assay Buffer. Add 175 μl of 1M DTT to 69825 μl of pre-made 2.5× Assay buffer without DTT.

PIP5K working solution: Prepare a suitable (e.g. for PIP5Kβ, 0.3 ng/μl) working solution of PIP5K in 2.5× assay buffer. For 20 plates you need 28.16 ml plus allow 4.52 ml extra as dead volume. For 20 plates add 867 μl of 11.3 μg/ml GST-PIP5K_{β} stock+31790 μl of 2.5× assay buffer. Immediately transfer 90 μl to Columns 1-22 of a Corning black polystyrene non-binding surface 384 well 90 μl microplate (Product code 3654). Add 90 μl of 2.5× Assay buffer to Columns 23-24. For 20 plate top-up add 768 μl of 11.3 μg/ml GST-PIP5K_{β} stock+28160 μl of 2.5× assay buffer.

Substrate/detection mixture: Prepare a mixture containing 20 μM PI(4)P+60 μM ATP+1 in 16 dilution of PH domain stock+20 nM F—PI(4,5)P₂ in miliQ water. For 20 plates you need 38.4 ml plus allow 7 ml extra as dead volume. For 20 plates add 908 μl of 1 mM PI(4)P stock+340.50 μl of 8 mM ATP stock+2837.5 μl of PH domain stock+90.8 μl of 10 μM F—PI(4,5)P₂ stock+41223.2 μl of miliQ water. For 20 plate top-up add 780 μl of 1 mM PI(4)P stock+292.5 μl of 8 mM ATP+2437.5 μl of PH domain stock+78 μl of 10 μM F—PI(4,5)P stock+35412 μl of miliQ water.

Perform the following additions into Black, Low volume, 384-well non-binding surface plates (Corning, Product code 3676) using a MATRIX PlateMate® Plus:
Add 4 μl of PIP5K working solution or 2.5× assay buffer to blanks.
Add 1 μl of compound in 20% DMSO or 20% DMSO to blanks and controls.
Add 5 μl of substrate/detection mixture.
Incubate for 1 hour at room temperature protected from light.
Read plates on an Analyst HTS plate reader using the settings described above.

### Plate Layout:

| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
|---|---|---|---|---|---|---|---|---|---|---|---|---|---|---|---|---|---|---|---|---|---|---|---|---|
| A | T | T | T | T | T | T | T | T | T | T | T | T | T | T | T | T | T | T | T | T | C | C | B | B |
| B | T | T | T | T | T | T | T | T | T | T | T | T | T | T | T | T | T | T | T | T | C | C | B | B |
| C | T | T | T | T | T | T | T | T | T | T | T | T | T | T | T | T | T | T | T | T | C | C | B | B |
| D | T | T | T | T | T | T | T | T | T | T | T | T | T | T | T | T | T | T | T | T | C | C | B | B |
| E | T | T | T | T | T | T | T | T | T | T | T | T | T | T | T | T | T | T | T | T | C | C | B | B |
| F | T | T | T | T | T | T | T | T | T | T | T | T | T | T | T | T | T | T | T | T | C | C | B | B |
| G | T | T | T | T | T | T | T | T | T | T | T | T | T | T | T | T | T | T | T | T | C | C | B | B |
| H | T | T | T | T | T | T | T | T | T | T | T | T | T | T | T | T | T | T | T | T | C | C | B | B |
| I | T | T | T | T | T | T | T | T | T | T | T | T | T | T | T | T | T | T | T | T | C | C | B | B |
| J | T | T | T | T | T | T | T | T | T | T | T | T | T | T | T | T | T | T | T | T | C | C | B | B |
| K | T | T | T | T | T | T | T | T | T | T | T | T | T | T | T | T | T | T | T | T | C | C | B | B |
| L | T | T | T | T | T | T | T | T | T | T | T | T | T | T | T | T | T | T | T | T | C | C | B | B |
| M | T | T | T | T | T | T | T | T | T | T | T | T | T | T | T | T | T | T | T | T | C | C | B | B |
| N | T | T | T | T | T | T | T | T | T | T | T | T | T | T | T | T | T | T | T | T | C | C | B | B |
| O | T | T | T | T | T | T | T | T | T | T | T | T | T | T | T | T | T | T | T | T | C | C | B | B |
| P | T | T | T | T | T | T | T | T | T | T | T | T | T | T | T | T | T | T | T | T | C | C | B | B |

Where
T = Test (Sub + Dr/DMSO + Protein)
C = Controls (Sub + DMSO + Protein)
B = Blanks (Sub only)

1.-66. (canceled)

67. A method for identifying an agent that modulates type I PIP kinase activity, or type II PIP kinase activity, the method comprising:
i. contacting type I PIP kinase or type II PIP kinase, or an active fragment of either thereof, with a test agent under conditions effective for type I PIP kinase activity or type II PIP kinase activity to occur in the absence of said test agent; and
ii. determining the level of activity of the type I PIP kinase or the level of activity of the type II PIP kinase, thereby to determine whether the test agent modulates the activity of type I PIP kinase or type II PIP kinase.

68. A method according to claim 67 wherein the method is for identifying an agent that inhibits activity.

69. A method according to claim 67 wherein:
(a) the method is for identifying an agent that modulates type I PIP kinase activity and wherein the test agent is contacted with a composition which comprises:
i. a fluorescently labelled product or product analogue of type I PIP kinase bound to phospholipase C or a fragment thereof;
ii. type I PIP kinase, or an active fragment thereof; and
iii. an unlabelled substrate of type I PIP kinase or unlabelled substrate analogue thereof; or
(b) the method is for identifying an agent that modulates type II PIP kinase activity and wherein the test agent is contacted with a composition which comprises:
i. a fluorescently labelled product or product analogue of type II PIP kinase bound to phospholipase C or a fragment thereof;
ii. type II PIP kinase, or an active fragment thereof; and
iii. an unlabelled substrate of type II PIP kinase or unlabelled substrate analogue thereof.

70. A method according to claim 69 wherein the fluorescently labelled product analogue of type I PIP kinase is selected from: PtdIns(3,5)P₂, PtdIns(4,5)P₂, and PtdIns(3,4,5)P₃ or the fluorescently labelled product analogue of type II PIP kinase is selected from: PtdIns(3,4)P₂, PtdIns(4,5)P₂, and PtdIns(3,4,5)P₃.

71. A method according to claim 69 wherein the type I PIP kinase substrate is selected from: PtdIns4P, PtdIns(3,4)P₂, PtdIns3P and PtdIns or an analogue thereof; or the type II PIP kinase substrate is selected from: PtdIns(3,5)P₂, PtdIns3P, PtdIns5P and PtdIns or an analogue thereof.

72. A method according to claim 69 wherein the labelled product or product analogue is bound to a fragment of phospholipase C comprising the pleckstrin homology (PH) domain.

73. A method according to claim 69 which further comprises:
i. contacting the agent identified with a mammalian cell; and
ii. determining the efficacy of the agent for a hyperproliferative or neurodegenerative disorder, said method comprising assaying the agent for a modulatory effect on:
a. cellular PtdIns(4,5)P₂ levels;
b. cellular apoptosis;
c. cellular sensitivity to apoptotic agents; and/or
d. neurite outgrowth.

74. A method according to claim 69 which is for identifying an agent for modulating apoptosis or neurite outgrowth in cells, or for treating a hyperproliferative disorder or a neurodegenerative disease.

75. A method for identifying an agent that modulates type I PIP kinase expression or type II PIP kinase expression, the method comprising:
i. providing a polynucleotide construct comprising a promoter of a gene encoding type I PIP kinase or type II PIP kinase, or a functional equivalent thereof, operably linked to a coding sequence;
ii. providing a test agent;
iii. contacting the test agent with the polynucleotide construct under conditions effective for expression of the polypeptide encoded by the coding sequence to occur in the absence of said test agent; and
iv. determining the level of expression of the polypeptide encoded by the coding sequence, thereby determining whether the test agent modulates the expression of the polypeptide.

76. A method of achieving an effect in a patient, comprising administering to said patient an agent selected from the group consisting of an agent that inhibits Type I PIP kinase expression and/or activity and an agent that inhibits Type II PIP kinase expression and/or activity, wherein said effect is inducing apoptosis or sensitizing cells to apoptotic agents, stimulating neurite outgrowth, or treating hyperproliferative disorders or neurodegenerative diseases.

77. A method according to claim 76 wherein the agent comprises an interfering RNA, antisense nucleic acid, a ribozyme or an antibody.

78. A method of achieving an effect in a patient, comprising administering to said patient an agent selected from the group consisting of an agent that enhances Type I PIP kinase expression and/or activity and an agent that enhances Type II PIP kinase expression and/or activity, wherein said effect is preventing apoptosis or treating cardiac reperfusion or neurodegenerative disease.

79. A method of achieving an effect in a patient, comprising administering to said patient an agent that modulates cellular levels of phosphatidylinositol-4,5-bisphosphate (PtdIns(4,5)P₂), wherein said effect is modulating apoptosis or cell sensitivity to apoptotic agents.

80. A method according to claim 79 wherein the agent decreases cellular levels of PtdIns(4,5)P₂ by inhibiting type I PIP kinase or type II PIP kinase or type I and type II PIP kinases and the effect is induction of cellular apoptosis or sensitization of the cell to apoptotic agents.

81. A method according to claim 79 wherein the agent decreases cellular levels of PtdIns(4,5)P₂.

82. A method according to claim 79 wherein the agent acts via:
i. an RNA interference (RNAi) mechanism; or
ii. an antisense mechanism.

83. A method according to claim 79 wherein the agent increases cellular levels of PtdIns(4,5)P₂ by stimulating or inducing expression and/or activity of type I or type II PIP kinase, or type I and type II PIP kinase and the effect is prevention of cellular apoptosis.

84. A method according to claim 83 wherein the agent increases cellular levels of PtdIns(4,5)P₂.

85. A method according to claim 83 which is for treating cardiac reperfusion or neurodegenerative disease.

86. A method of achieving an effect in a patient, comprising administering to said patient an agent that decreases cellular levels of phosphatidylinositol-4,5-bisphosphate (PtdIns(4,5)P₂), wherein said effect is stimulating neurite outgrowth or combating a hyperproliferative disorder in the patient.

87. A method according to claim 86 which is for treating a neurodegenerative disorder.

88. A method according to claim 79 wherein the agent decreases cellular levels of PtdIns(4,5)P₂ by inhibiting type I PIP kinase or type II PIP kinase or type I and type II PIP kinases.

89. A method according to claim 83 wherein the agent acts via:
i. an RNA interference (RNAi) mechanism; or
ii. an antisense mechanism.

90. A method of achieving an effect in a patient, comprising administering to said patient an agent that increases cellular levels of PtsIns4P and/or PtsIns5P, wherein said effect is inducing cellular apoptosis or sensitising a cell to an apoptotic agent.

91. A method according to claim 90 wherein cellular levels of PtsIns4P or PtsIns5P are increased by inhibiting type I PIP kinase or type II PIP kinase or type I and type II PIP kinase.


SEQUENCE LISTING (22 sequences)

SEQ ID NO: 1 - protein, 23 aa, Artificial Sequence
  Synthetic Sequence
RRCIFALAVRKGGALKKGAIAKT

SEQ ID NO: 2 - protein, 25 aa, Artificial Sequence
  Synthetic Sequence
PKCRGDNEKTMDKSTEKTKKERRAR

SEQ ID NO: 3 - protein, 26 aa, Artificial Sequence
  Synthetic Sequence
PKCRGESEKTMDKALEKSKKERAYNR

SEQ ID NO: 4 - protein, 23 aa, Artificial Sequence
  Synthetic Sequence
DCCQRAPPTPRKVGRRGKNSKEG

SEQ ID NO: 5 - protein, 15 aa, Artificial Sequence
  Synthetic Sequence
PQCTAAMKRRGSRHK

SEQ ID NO: 6 - protein, 42 aa, Artificial Sequence
  Synthetic Sequence
RKHKKTAHNSEADLEESFNEHELEPSSPKTKKKSRKGRPRKT

SEQ ID NO: 7 - protein, 21 aa, Artificial Sequence
  Synthetic Sequence
PKCANKIKKDKKHKKRKHRAH

SEQ ID NO: 8 - protein, 37 aa, Artificial Sequence
  Synthetic Sequence
RECREKDPKLEIRYRHKKCRERDGSERAGSEPRDEGG

SEQ ID NO: 9 - protein, 24 aa, Artificial Sequence
  Synthetic Sequence
KNHSGNDERDEEDEERESKSRGRV

SEQ ID NO: 10 - protein, 57 aa, Artificial Sequence
  Synthetic Sequence
LECTVCEACGKATDPGRLLLCDDCDISYHTYCLDPPLQTVPKGGWKCKWCVWCRHCG

SEQ ID NO: 11 - protein, 36 aa, Artificial Sequence
  Synthetic Sequence
SECDQAGSSDMEAEMAMETLPDGTKRSRRQIKEPVK

SEQ ID NO: 12 - protein, 25 aa, Artificial Sequence
  Synthetic Sequence
SVCNQGPEYIERLPLRWVDIVHLAL

SEQ ID NO: 13 - protein, 416 aa, Rattus norvegicus
MSSNCTSTTAVAVAPLSASKTKTKKKHFVCQKVKLFRASEPILSVLMWGVNHTINELSNVPVPVMLMPDDFKAYSKIKVDNHLFNKENLPSRFKFKEYCPMVFRNLRERFGIDDQDYQNSVTRSAPINSDSQGRCGTRFLTTYDRRFVIKTVSSEDVAEMHNILKKYHQFIVECHGNTLLPQFLGMYRLTVDGVETYMVVTRNVFSHRLTVHRKYDLKGSTVAREASDKEKAKDLPTFKDNDFLNEGQKLRVGEESKKNFLEKLKRDVEFLAQLKIMDYSLLVGIHDVDRAEQEETEVEDRAEEEECENDGVGGGLLCSYGTPPDSPGNLLSFPRFFGPGEFDPSVDVYAMKSHESAPKKEVYFMAIIDILTPYDAKKKAAHAAKTVKHGAGAEISTVNPEQYSKRFNEFMSNILT

SEQ ID NO: 14 - DNA, 1987 bp, Rattus norvegicus
gcgctccgtgagcagcgggggtggggatcggttcgggggaggcctgggccgtctgctggtgcgctgactccgccgccccccctttcgccgccgccgccgccgccgccccgggcatgtcgtccaactgcaccagcaccacggcggtggcggtggcgccgctcagcgccagcaaaaccaagaccaagaagaagcatttcgtgtgccagaaagtgaagctattccgggccagcgagccgattctcagcgtcctgatgtggggggtgaaccacacgatcaatgagctgagcaatgtccccgtccctgtcatgctaatgccagatgacttcaaagcttacagcaagatcaaggtggacaatcatctcttcaataaggaaaacctccccagccgcttcaagttcaaggaatactgccccatggtgttccgaaacctcagggagaggtttggcatcgatgaccaggattaccagaattcagtgacacgcagtgcgcccatcaacagtgacagccagggcaggtgcggcacacgcttcctcaccacctatgaccggcgttttgtcatcaagactgtgtcaagtgaggatgtggcagagatgcacaacatcctaaagaaataccaccagttcatagtggagtgtcacggcaataccctcttgccacagttcctgggcatgtaccgcctgaccgtggacggtgtggaaacctacatggtggtcaccaggaatgtattcagtcatcggcttactgtacatcgcaaatatgacctcaagggttccaccgttgccagagaagccagtgacaaggaaaaggccaaggacttgcccacgttcaaagacaatgacttcctcaatgaagggcagaagctacgtgtgggagaagagagtaaaaagaacttcctagaaaaactgaagcgggatgtggagttcctggcccagctgaagatcatggactacagcctcctggtgggcatccacgacgtagaccgggcagagcaggaggagacggaggtggaagaccgagcggaggaggaggagtgtgagaacgatggggtgggcggtggcctgctctgctcctacggcacgcctccagacagccccggcaacctcctgagcttcccccggttctttggcccaggagagttcgacccctccgtcgacgtctatgccatgaaaagccatgagagtgcccctaagaaggaggtctatttcatggccatcattgacatccttacaccgtatgacgctaagaagaaagctgcacatgccgccaagacagtgaaacatggggcaggggctgagatctccactgtgaacccggagcagtactcaaaacgcttcaatgagttcatgtccaacatcctgacgtagtcgtctctaccttctgccagagccagactgctggacaaggggtcggggatcgggagagggaaagggtgtatttgggctggaagggagggcggggagcagagtcggggctcatgagggctttagtaaagggactgcagcctgtgacacaaagcaactctagaaaggaagaaggagctggttgttggacacgtgtcacagcgtggcagccttacttccccagcaccctggatgcgccccttttgtccctgggttaaaacaaggtttcctcttcggtatctttgtaaccctttatgatcctaggggctggagttgactttgtgggttgatttgggtttgtttcttaaattcattgctccaggatcgctctttataacgctgtatgtcatcaccccaggcaccctccctattctggtcaggctctcagtgcccttcaactaaggccagcacaagggacgtgaacagcacttgctctgacaccatgagaggtcagacacctcagtcagcgacgttttaatctcccctcgatggactttttattttctggagtattgtgcataattccctccatgtctccaccttggatccaagtgaaacattacattggctccttcatccctcgtgcc

SEQ ID NO: 15 - protein, 416 aa, Homo Sapiens
MSSNCTSTTAVAVAPLSASKTKTKKKHFVCQKVKLFRASEPILSVLMWGVNHTINELSNVPVPVMLMPDDFKAYSKIKVDNHLFNKENLPSRFKFKEYCPMVFRNLRERFGIDDQDYQNSVTRSAPINSDSQGRCGTRFLTTYDRRFVIKTVSSEDVAEMHNILKKYHQFIVECHGNTLLPQFLGMYRLTVDGVETYMVVTRNVFSHRLTVHRKYDLKGSTVAREASDKEKAKDLPTFKDNDFLNEGQKLHVGEESKKNFLEKLKRDVEFLAQLKIMDYSLLVGIHDVDRAEQEEMEVEERAEDEECENDGVGGNLLCSYGTPPDSPGNLLSFPRFFGPGEFDPSVDVYAMKSHESSPKKEVYFMAIIDILTPYDTKKKAAHAAKTVKHGAGAEISTVNPEQYSKRFNEFMSNILT

SEQ ID NO: 16 - DNA, 3743 bp, Homo Sapiens
ttgcgggaaagagccaaaccctggcgttggggggcccgggcggggagcccctcccgcggtccacagcgacgcctgcccagccctcctccccttccggctccggcacggggccccgaggcgttcggaggccaggcgggtttctgtcaggcccggggaggaggggcgggcggggcggccgctgcctccccgggacgggccgtaccacgcggacggggaggacggggccaggggactgcagggcggctgcaccgcccgggggcggggtgcggacgggccggcgggctccccggggcggggcgggagggcggggcgtggggcggacggaaccaccggggcggggtgggaggtaacgggacgggcgcgaccatggcgcggtgagggagcgggggtggggatcggtccgggggaggcctgaggccgctggcttgtgcgctgtctccgccgcccccctctttcgccgccgccgccgccgccccgggcatgtcgtccaactgcaccagcaccacggcggtggcggtggcgccgctcagcgccagcaagaccaagaccaagaagaagcatttcgtgtgccagaaagtgaagctattccgggccagcgagccgatcctcagcgtcctgatgtggggggtgaaccacacgatcaatgagctgagcaatgttcctgttcctgtcatgctaatgccagatgacttcaaagcctacagcaagatcaaggtggacaatcatctcttcaataaggagaacctgcccagccgctttaagtttaaggagtattgccccatggtgttccgaaaccttcgggagaggtttggaattgatgatcaggattaccagaattcagtgacgcgcagcgcccccatcaacagtgacagccagggtcggtgtggcacgcgtttcctcaccacctacgaccggcgctttgtcatcaagactgtgtccagcgaggacgtggcggagatgcacaacatcttaaagaaataccaccagtttatagtggagtgtcatggcaacacgcttttgccacagttcctgggcatgtaccgcctgaccgtggatggtgtggaaacctacatggtggttaccaggaacgtgttcagccatcggctcactgtgcatcgcaagtatgacctcaagggttctacggttgccagagaagcgagcgacaaggagaaggccaaggacttgccaacattcaaagacaatgacttcctcaatgaagggcagaagctgcatgtgggagaggagagtaaaaagaacttcctggagaaactgaagcgggacgttgagttcttggcacagctgaagatcatggactacagcctgctggtgggcatccacgacgtggaccgggcagagcaggaggagatggaggtggaggagcgggcagaggacgaggagtgtgagaatgatggggtgggtggcaacctactctgctcctatggcacacctccggacagccctggcaacctcctcagctttcctcggttctttggtcctggggaattcgacccctctgttgacgtctatgccatgaaaagccatgaaagttcccccaagaaggaggtgtatttcatggccatcattgatatcctcacgccatacgatacaaagaagaaagctgcacatgctgccaaaacggtgaaacacggggcaggggccgagatctcgactgtgaaccctgagcagtactccaaacgcttcaacgagtttatgtccaacatcctgacgtagttctcttctaccttcagccgagaccgagagactggatatggggtcggggatcgggacttagggagaagggtgtatttgggctagatgggagggtgggagcgagatcgggtttgggagggctttagcaatgagacttgcagcctgtgacaccgaaagagactttagctgaagaggagggggatgtgctgtgtgtgcaccagctcacaggatgtaaccccaccttctgcttacccttgattttttctccccatttgacacccaggttaaaaaggggttccctttttggtaccttgtaaccttttaagataccttggggctagagatgacttcgtgggtttatttgggttttgtttctgaaatttcattgctccaggtttgctatttataatcatatttcatcagcctacccaccctccccatctttgctgctctcagttcccttcaattaaagagatacccagtagacccagcacaagggtccttccagaaccaagtgctatggatgccagattggagaggtcagacacctcgccctgctgcatttgctcttgtctggattaactttgtaatttatggagtattgtgcacaacttcctccacctttcccttggattcaagtgaaaactgttgcattattcctccatcctgtctggaatacaccaggtcaacaccagagatctcagatcagaatcagagatctcagaggggaataagttcatcctcatgggatggtgaggggcaggaaagcggctgggctcttggacaccctggttctcagagaaccctgtgatgatcacccaagccccaggctgtcttagcccctggagttcagaagtcctctctgtaaatcctgcctcccactaggtcaagaggaactagagtacctttggatttatcaggaccctcatgtttaaatggttatttccctttgggaaaacttcagaaactgatgtatcaaatgaggccctgtgccctcgatctatttccttcttccttctgacctcctcccaggcactcttacttctagccgaactcttagctctgggcagatctccaagcgcctggagtgctttttagcagagacacctcgttaagctccgggatgaccttgtaggagatctgtctcccctgtgcctggagagttacagccagaaaggtgcccccatcttagagtgtggtgtccaaacgtgaggtggcttcctagttacatgaggatgtgatccaggaaatccagtttggaggcttgatgtgggttttgacctggcctcaccttggggctgtttttccttgttgccccgctctagacttttagcagatctgcacccacaggcttttttggaaggagtggcttcctcgaggtgttccacctgcttcggagcctgccacccaggccctcagaactgaccacaggctgctctggccaggagagaaacagctctgttgttctgcattgggggaggtacattcctgcatcttctcaccccctcaaccaggaactggggatttgggatgagatatggtcagacttgtagataaccccaaagatgtgaagatcgcttgtgaaaccattttgaatgaatagattggtttcctgtggctccctccaaacctggccaagcccagcttccgaagcaggaaccagcactgtctctgtgcctgactcacagcatataggtcaggaaagaatggagacggcattcttggacttcactggggctgctggattggatgggaaaccttctggaagaggcagatgggggtcaaaccactgccttgccccaggaaggggccataggtaggtctgaacaactgccggaagaccactacatgacttagggaacttgaaaccaactggctcatggagaaaacaaatttgacttgggaaagggattatgtaggaataatgtttggacttgatttccccacgtcataatgaagaatggaagtttggatctgctcctcgtcaggcgcagcatctctgaagcttggaaagctgtcttccagggttgtaa

SEQ ID NO: 17 - protein, 539 aa, Mus musculus
MSSTAENGDAVPGKQNEEKTYKKTASSAIKGAIQLGIGYTVGNLTSKPERDVLMQDFYVVESVFLPSEGSNLTPAHHYPDFRFKTYAPLAFRYFRELFGIKPDDYLYSICSEPLIELSNPGASGSLFFLTSDDEFIIKTVQHKEAEFLQKLLPGYYMNLNQNPRTLLPKFYGLYCMQSGGINIRIVVMNNVLPRAMRMHLTYDLKGSTYKRRASRKEREKPNPTFKDLDFLQDMHEGLYFDTETYNALMKTLQRDCRVLESFKIMDYSLLLGIHILDHSLKDKEEEPLQNVPDAKRPGMQKVLYSTAMESIQGPGKSADGIIAENPDTMGGIPAKSHKGEKLLLFMGIIDILQSYRLMKKLEHSWKALVYDGDTVSVHRPSFYADRFLKFMNSRVFKKIQALKASPSKKRCNSIAALKATSQEIVSSISQEWKDEKRDLLTEGQSFSSLDEEALGSRHRPDLVPSTPSLFEAASLATTISSSSLYVGEHYPHDRTTLYSNSKGLPSSSTFTLEEGTIYLTAEPNTLDLQDDASVLDVYL

SEQ ID NO: 18 - DNA, 2378 bp, Mus musculus
cctgggggaacaaaagcagtgtcaacgatgagcagaccacccgtcaccaccaactccagatgacttgccattttccttacaaagatgtcgtcaactgctgaaaatggagacgccgtacctggaaaacaaaacgaagaaaaaacctacaaaaagacggcatcgtcagctattaaaggtgctatccagctgggaataggatacacagtgggcaacctcacgtccaagccagaacgcgatgtcctcatgcaagacttctacgtggtggaaagtgtgttcctccccagtgaagggagcaacctgaccccagcacatcactacccagacttcaggtttaagacctacgcaccgctggccttccggtacttccgagaactctttggtatcaagcccgatgattacttgtactccatctgcagtgaacctctgatagaactgtccaatcctggagccagtgggtccttgtttttcttgaccagcgatgatgaatttatcatcaaaaccgttcagcataaggaagccgagttcctgcagaagctgctgccgggctattacatgaatttaaaccagaatccaaggactcttctgccaaaattctatgggctgtattgcatgcagtcaggaggcatcaacatccgtattgtggtgatgaacaatgtcctgccgcgtgccatgaggatgcatttgacctatgacctgaaaggctccacatacaagcgaagagcatcccgaaaagagagggagaaacccaaccccacgtttaaggacctggacttcctgcaggacatgcacgaggggctgtatttcgatacagaaacatacaatgcccttatgaagacactacagagagactgccgggttctggaaagcttcaagatcatggactacagccttttgctgggtatccacatcttggaccattccctcaaagacaaagaagaggagcccctccaaaacgtgcctgatgcaaagcggccggggatgcagaaagtcctctattccacagccatggaatccatccagggtccaggcaagtctgcagatgggatcattgcagagaatccagacacaatgggaggcattccagctaaaagccataagggagagaagctgcttctattcatgggcattattgacatcctccaatcatataggttaatgaagaagttagaacattcctggaaagcccttgtttatgatggggacaccgtttcagttcatagaccaagtttttatgcagatagatttttaaagtttatgaattccagagttttcaagaaaattcaagctctgaaagcctcgccttctaagaaacgctgcaactccatcgctgcactgaaggcaacctcccaggagatcgtatcctccatcagccaggaatggaaggacgagaagcgggatttgttaaccgaaggacagagtttcagcagcctggatgaagaagccctgggatcccgacacaggcccgacctggtacccagcactccatcattatttgaagctgcttccttagcgaccacaatatcatcttcttccttatatgtcggcgaacactatccacatgacaggactaccctttactcaaacagcaaagggttaccttccagttcaaccttcaccttggaagaggggaccatctacctgacggctgagccaaacaccctggacctgcaggatgatgcctctgtgctggacgtctatttataagtgaaagcggcagccacctgagcacgtgagcgggacctgaacacagccatgggagggaacttcccccactgcagaattcctccagaagaactgggctgaaccccagcacacacagtaagactcagctggaccttgaagcccgcaagatgccggggagcctcggtccgtggccgatccgcagatggatgtgaaaatcccacaccgttccgtgggtgctgctgcttgctgaccatgaagacctgcatgacctctgtctaagccgcttttctacaccatggccaatcacccttttggactgggaagcgctattttcctataggctctctcggtgtttcattctgcatgcatagagtgattacaaataggcaacctatggaatctgcttctatatttttatataaaaaaaatctatccgtgcaaaatgataaataagatctaaatcctctctctttgtttcagagattctaatcatagcccctgtcattgaaagcttcaatctgttttcctttaaagatcagtatactaggtctgcctttactttatagaaagccagcttctataaagatttttttcactgtttactagtgaaatgagaaaaagcaaagctatttataaaaggccttatgtcttgtatatacattgtctttggaatatttgtgatttagtttattgct

SEQ ID NO: 19 - protein, 549 aa, Homo Sapiens
MASASSGPSSSVGFSSFDPAVPSCTLSSASGIKRPMASEVPYASGMPIKKIGHRSVDSSGETTYKKTTSSALKGAIQLGITHTVGSLSTKPERDVLMQDFYVVESIFFPSEGSNLTPAHHYNDFRFKTYAPVAFRYFRELFGIRPDDYLYSLCSEPLIELCSSGASGSLFYVSSDDEFIIKTVQHKEAEFLQKLLPGYYMNLNQNPRTLLPKFYGLYCVQAGGKNIRIVVMNNLLPRSVKMHIKYDLKGSTYKRRASQKEREKPLPTFKDLDFLQDIPDGLFLDADMYNALCKTLQRDCLVLQSFKIMDYSLLMSIHNIDHAQREPLSSETQYSVDTRRPAPQKALYSTAMESIQGEARRGGTMETDDHMGGIPARNSKGERLLLYIGIIDILQSYRFVKKLEHSWKALVHDGDTVSVHRPGFYAERFQRFMCNTVFKKIPLKPSPSKKFRSGSSFSRRAGSSGNSCITYQPSVSGEHKAQVTTKAEVEPGVHLGRPDVLPQTPPLEEISEGSPIPDPSFSPLVGETLQMLTTSTTLEKLEVAESEFTH

SEQ ID NO: 20 - DNA, 3713 bp, Homo Sapiens
attaacaggccgtggttaggaaggacggagaaggggcgttcgctcctttgggacttttcatgcctcgtttttttttcagatgtggcttggtctgggcgcaaggtcccagcagccagcttaagcttactcttctgtgaaaggggaaagtatcccctgtggaaagcggttaaacttgtggagggggtgcgggacgtgagttcttccccatgccaggcgaatggtgtggccttgagctggtccaggagccggctcgacgtgtctgagggaggcccggagggggcggggaggtggcccacagaacgcgggttctgtaaagagacgttgggaagattcgattccgagaagaggaagaaccggattgaaagagagccaggccgctgagggggagggggctgctaagatggcgtcggcctcctccgggccgtcgtcttcggtcggtttttcatcctttgatcccgcggtcccttcctgtaccttgtcctcagcatctggaatcaagagacccatggcatctgaggtgccttatgcctctggcatgcccatcaagaaaataggccatagaagtgttgattcctcaggagagacaacatataaaaagacaacctcatcagccttgaaaggtgccatccagttaggcattacccacactgtggggagcctgagtaccaaaccagagcgtgatgtcctcatgcaagatttctacgtggttgagagtatcttctttcccagtgaagggagcaacctgacccctgctcatcactacaatgactttcgtttcaagacctatgcacctgttgccttccgctacttccgggagctatttggtatccggcccgatgattacttgtattccctctgcagtgagccgctgattgaactctgtagctctggagctagtggttccctattctatgtgtccagcgacgatgagttcattattaagacagtccaacataaagaggcggaatttctgcagaagctgcttccaggatactacatgaacctcaaccagaaccctcggactttgctgcctaaattctatggactgtactgtgtgcaggcaggtggcaagaacattcggattgtggtgatgaacaatcttttaccaagatcggtaaaaatgcatatcaaatatgacctcaaaggctcaacctacaaacggcgggcttcccagaaagagcgagagaagcctcttcccacatttaaagacctagacttcttacaagacatccctgatggtctttttttggatgctgacatgtacaacgctctctgtaagaccctgcagcgtgactgtttggtgctgcagagcttcaagataatggattacagcctcttgatgtcaatccataatatagatcatgcacaacgagagcccttaagcagtgaaacacagtactcagttgatactcgaagaccggccccccaaaaggctctgtattccacagccatggaatccatccagggagaggctcgacggggtggtaccatggagactgatgaccatatgggtggcatccctgcccggaatagtaaaggggaaaggcttctgctttatattggcatcattgacattctacagtcttacaggtttgttaagaagttggagcactcttggaaagccctggtacatgacggagacactgtctcagtgcatcgcccaggcttctacgctgaacggttccagcgcttcatgtgcaacacagtatttaagaagattcccttgaagccttctccttccaaaaagtttcggtctggctcatctttctctcggcgagcaggctccagtggcaactcctgcattacttaccagccatcggtctctggggaacacaaggcacaagtgacaacaaaggcagaagtggagccaggcgttcaccttggtcgtcctgatgttttacctcagactccacctttggaggaaatcagtgagggctcgcctattcctgaccccagtttctcacctctagttggagagactttgcaaatgctaactacaagtacaaccttggaaaagcttgaagttgcagagtcagagttcacccattaagcgcaaagcctcagaagacctggaacaagattctgccatctctgtgatcccaagatgtcagcccttgccccagcaatgctgaattttcttctacttggtcatcaaaaaaggagtgtaatagaagtgaggggagctgctcctccatcttcttcctgaagaagaaccttctctccttcctcttcctcatgaatgggccttagtgcctcagagagttgaggaccgcagcatcccctccactccagagttgggtggtacggattttcaactggccaaccctttgcctccactattgaatttttttcagacccccattcttcatgctggaaatgggattgctggacttggcagctttctttcccctcgtctttgactaggaaccggactcttaatttcctcaggacagactagctggcacattatccctaccttagttctttctctctgactcctggaagaatactcctgtaatctctgtaaaggtttttgggggataagggtgtttaaccacctcccagctttcttcttcttttttttttctgaaaaaaggaaaaagcacacagcacacaatttcaagccattttcagatcagaactccagaagtgttgacaagatgcctattcgtagagttccctcagaagagccatggtgtttatgaagagaagagtagtgattgctctgccagaagcagctcctctttaaactcctcctctcttgatgaatttcttaaggctgaaggaatgaagagagtgggacatggggtaatctttatcccttttgttaaaacaggaggcagccatgggctgggagatcatagcccttcctaggcagaatcctgttcactgccaggctatagtaattattactattttgcaatttgaaatatattctggttgtttttctaaatgtgaagacttaccaaatgaattttagatcattctccagaggagattttttttgctcttctcatcttttccaacagtgttctcctgtttgtggagctaaggtaaagaggggacacttctgtctgtttaacagacagtccatatctgtgaggccagcaaatattttcttaaactcatggggagacagcagattcttgccttggtgaggtcattgctgtgccatatgtcctacccccctgtcttcatgcagggaagttggaaatgggggctacatatgccctctcctccccgtctacaagagttgtggttttccatctgatccttccactcttgtcaggggaagaagggggcctggtatctcaggcagattgttgaattcctgttctatcccttctctatcccaccctgccttgataatatgttagcccataccccaaataactgtctatattagacacccccagccagtttctggctgcctgtctttgctgccatgttttttacaagaaggaaagaattcttgctatttttttttcataatttactatttatgatgtatttaagtgttttattaaggacagagttctgttaggggtgggagggaatatttgagggagggctgggtcttagggaaaggaatggggaagcaacatttttattaagtgttactatttgcctctactttgtattgttcagaaatggcaaatacaatataaaagtgatatatggttttaatgtaataaactttaatgagttattta

SEQ ID NO: 21 - protein, 756 aa, Homo sapiens
MDSGRDFLTLHGLQDDEDLQALLKGSQLLKVKSSSWRRERFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLKELNIQVDDSYARKIFRECDHSQTDSLEDEEIEAFYKMLTQRVEIDRTFAEAAGSGETLSVDQLVTFLQHQQREEAAGPALALSLIERYEPSETAKAQRQMTKDGFLMYLLSADGSAFSLAHRRVYQDMGQPLSHYLVSSSHNTYLLEDQLAGPSSTEAYIRALCKGCRCLELDCWDGPNQEPIIYHGYTFTSKILFCDVLRAIRDYAFKASPYPVILSLENHCTLEQQRVMARHLHAILGPMLLNRPLDGVTNSLPSPEQLKGKILLKGKKLGGLLPPGGEGGPEATVVSDEDEAAEMEDEAVRSRVQHKPKEDKLRLAQELSDMVIYCKSVHFGGFSSPGTPGQAFYEMASFSENRALRLLQESGNGFVRHNVGHLSRIYPAGWRTDSSNYSPVEMWNGGCQIVALNFQTPGPEMDVYQGRFQDNGACGYVLKPAFLRDPNGTFNPRALAQGPWWARKRLNIRVISGQQLPKVNKNKNSIVDPKVTVEIHGVSRDVASRQTAVITNNGFNPWWDTEFAFEVVVPDLALIRFLVEDYDASSKNDFIGQSTIPLNSLKQGYRHVHLMSKNGDQHPSATLFVKISLQD

SEQ ID NO: 22 - DNA, 2674 bp, Homo sapiens
ccgcactccgctcggaccccaggccgccggtgctgtcgctactcaagtgagtcccgcggtgcccctcccgccgcgccgcccgtcgggcatggactcgggccgggacttcctgaccctgcacggcctacaggatgatgaggatctacaggcgctgctgaagggcagccagctcctgaaggtgaagtccagctcatggaggagagagcgcttctacaagttgcaggaggactgcaagaccatctggcaggagtcccgcaaggtcatgcggaccccggagtcccagctgttctccatcgaggacattcaggaggtgcgaatggggcaccgcacggagggtctggagaagttcgcccgtgatgtgcccgaggaccgctgcttctccattgtcttcaaggaccagcgcaatacactagacctcatcgccccatcgccagctgatgcccagcactgggtgctggggctgcacaagatcatccaccactcaggctccatggaccagcgtcagaagctacagcactggattcactcctgcttgcgaaaagctgacaaaaacaaggacaacaagatgagcttcaaggagctgcagaacttcctgaaggagctcaacatccaggtggacgacagctatgcccggaagatcttcagggagtgtgaccactcccagacagactccctggaggacgaggagattgaggccttctacaagatgctgacccagcgggtggagatcgaccgcaccttcgccgaggccgcgggctcaggggagactctgtcggtggatcagttagtgacgttcctgcagcaccagcagcgggaggaggcggcagggcctgcgctggccctctccctcattgagcgctacgagcccagcgagactgccaaggcgcagcggcagatgaccaaggacggcttcctcatgtacttactgtcggctgacggcagcgccttcagcctggcacaccgccgtgtctaccaggacatgggccagccacttagccactacctggtgtcctcttcacacaacacctacctgctggaggaccagctagccgggcccagcagcactgaagcctacatccgggcactgtgcaaaggctgccgatgcctggagcttgactgctgggacgggcccaaccaggaaccaatcatctaccacggctatactttcacttccaagatcctcttctgcgatgtgctcagggccatccgggactatgccttcaaggcgtccccctaccctgtcatcctatccctggagaaccactgcacactggagcagcagcgcgtgatggcgcggcacctgcatgccatcctgggccccatgctgttgaaccgaccactggatggggtcaccaacagcctgccctcccctgagcaactgaaggggaagatcctgctgaaggggaagaagctcggggggctcctgccccctggaggggagggtggccctgaggccactgtggtgtcagacgaagacgaggctgctgagatggaggatgaggcagtgaggagccgtgtgcagcacaagcccaaggaggacaagctcaggctagcacaggagctctctgacatggtcatttactgcaagagtgtccactttgggggcttctccagtcctggcacccctggacaggccttctacgagatggcgtccttctctgagaaccgtgcccttcgactgctccaagaatcaggaaacggctttgtccgccacaacgtggggcacctgagcagaatctacccggctggatggagaacagactcctccaactacagccccgtggagatgtggaatgggggctgccagatcgtggccctgaatttccagacacctgggccagagatggacgtgtaccagggccgcttccaggacaacggggcctgtgggtacgtgctgaagcccgccttcctgcgagaccccaacggcacctttaacccccgcgccctggctcaggggccctggtgggcacggaagcggctcaacatcagggtcatttcggggcagcagctgccaaaagtcaacaagaataagaattcaattgtggaccccaaagtgacagtggagatccatggcgtgagccgggacgtggccagccgccagactgctgtcatcaccaacaatggtttcaacccatggtgggacacggagtttgcgtttgaggtagttgtgcctgaccttgccctcatccgcttcttggtggaagattatgatgcctcctccaagaatgacttcattggccagagtaccatccccttgaacagcctcaagcaaggataccgccatgtccacctcatgtctaagaacggggaccagcatccatcagccaccctctttgtgaagatctccctccaggactaggctggaggaagccagtggggtcccccctgagtgggctgggccctctgtccacatgtggggacagggctggtgtggctgctcccagcctcttgctcagagctaggcccccaaattgccttcagccctaacatagtgtctgctgctgcctccctggggaccaggagctagcccagtccctggagctgtccttcattccgttaggaataacactgcagccctctccaccctccggccagcgagtggtcaaggatttttataaaaatcacgataagattaaaaaaaaaaaaaaaaaaaaaaaaaaaaaaaaaaaaaaaa